(19) United States
(12) Patent Application Publication
(10) Pub. No.: US 2013/0172333 A1
(43) Pub. Date: Jul. 4, 2013

(54) FORMULATION AND DOSING OF HSP90 INHIBITORY COMPOUNDS

(71) Applicants: Neera Jain, Burlington, MA (US); Suresh R. Babu, Superior Township, MI (US); Shweta Kotecha, Reading, MA (US); Hong Yang, Lexington, MA (US)

(72) Inventors: Neera Jain, Burlington, MA (US); Suresh R. Babu, Superior Township, MI (US); Shweta Kotecha, Reading, MA (US); Hong Yang, Lexington, MA (US)

(73) Assignee: SYNTA PHARMACEUTICALS CORP., Lexington, MA (US)

(21) Appl. No.: 13/698,867

(22) PCT Filed: May 20, 2011
(86) PCT No.: PCT/US2011/037283

§ 371 Date: Mar. 14, 2013

Related U.S. Application Data
(60) Provisional application No. 61/346,769, filed on May 20, 2010.

(51) Int. Cl. A61K 31/4196; A61K 31/5377; A61K 31/52; A61K 31/423; A61K 31/4709; A61K 31/4439; A61K 31/428; A61K 31/538; A61K 31/4725

(52) U.S. Cl. CPC A61K 31/4196; A61K 31/538; A61K 31/5377; A61K 31/52; A61K 31/4725; A61K 31/4709; A61K 31/4439; A61K 31/428; A61K 31/423
USPC 514/230.5; 514/236.2; 514/263.2; 514/310; 514/314; 514/340; 514/367; 514/375; 514/384

(57) ABSTRACT

Provided is a pharmaceutical composition comprising a pharmaceutically acceptable organic solvent, a pharmaceutically acceptable surfactant, and a compound according to the following formula: wherein the variables are defined herein. Optionally, the pharmaceutical composition further comprises a co-solvent. Also provided is a method of using the pharmaceutical composition disclosed herein for the treatment of a patient in need thereof.

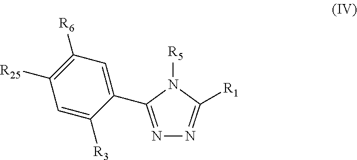

### CROSS-REFERENCE TO RELATED PATENT APPLICATIONS

This application claims the benefit of U.S. Provisional Application No. 61/346,769, filed on May 20, 2010, the entire disclosure of which is incorporated herein by reference.

### BACKGROUND OF THE INVENTION

Cancer is a group of diseases characterized by dysregulation of cell differentiation and proliferation and, in advanced stages, spread to other areas of the body including vital organs and bone. If not brought under control, these diseases can be fatal.

Through advancements in detection, surgery and therapeutic options, especially in the area of targeted therapies, patients' prognoses are generally improving, and 5-year survival rates for a number of cancers are rising. Nevertheless, the room for continued improvement in treatment options is vast: the American Cancer Society estimates approximately 1.4 million new cases of cancer will be diagnosed in the US this year, with 564,830 cancer-related deaths in 2006 in the US, and about 10 times this number worldwide (cancer.org).

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is unlikely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Examples of Hsp90 client proteins that have been implicated in the progression of cancer are described below.

Her2 is a transmembrane tyrosine kinase cell surface growth factor receptor that is expressed in normal epithelial cells. Her2 has an extracellular domain that interacts with extracellular growth factors and an internal tyrosine kinase portion that transmits the external growth signal transduction pathways leading to cell growth and differentiation. Her2 is overexpressed in a significant proportion of malignancies, such as breast cancer, ovarian cancer, prostate cancer and gastric cancers, and is typically associated with a poor prognosis. It is encoded within the genome by HER2/neu, a known proto-oncogene. HER2 is thought to be an orphan receptor, with none of the EGF family of ligands able to activate it. However, ErbB receptors dimerise on ligand binding, and HER2 is the preferential dimerisation partner of other members of the ErbB family. The HER2 gene is a proto-oncogene located at the long arm of human chromosome 17 (17q21-q22). HER2/neu (also known as ErbB-2) stands for “Human Epidermal growth factor Receptor 2” and is a protein giving higher aggressiveness in breast cancers. It is a member of the ErbB protein family, more commonly known as the epidermal growth factor receptor family. HER2/neu has also been designated as CD340 (cluster of differentiation 340) and p185. Approximately 15-20 percent of breast cancers have an amplification of the HER2/neu gene or overexpression of its protein product. Overexpression of this receptor in breast cancer is associated with increased disease recurrence and worse prognosis.

Anaplastic Lymphoma Kinase (ALK) tyrosine kinase receptor is an enzyme that in humans is encoded by the ALK gene. The 2;5 chromosomal translocation is frequently associated with anaplastic large cell lymphomas (ALCLs). The translocation creates a fusion gene consisting of the ALK (anaplastic lymphoma kinase) gene and the nucleophosmin (NPM) gene: the 3′ half of ALK, derived from chromosome 2, is fused to the 5′ portion of NPM from chromosome 5. The product of the NPM-ALK fusion gene is oncogenic. Other possible translocations of the ALK gene, such as the elm4 translocation, are also implicated in cancer.

B-Raf proto-oncogene serine/threonine-protein kinase (B-RAF), also known as V-raf murine sarcoma viral oncogene homolog B1, is a protein that in humans is encoded by the BRAF gene. The B-RAF protein is involved in sending signals in cells and in cell growth. The BRAF gene may be mutated, and the B-RAF protein altered, as an inherited mutation which causes birth defects, or as an acquired mutation (oncogene) in adults which causes cancer. Acquired mutations in this gene have also been found in cancers, including non-Hodgkin lymphoma, colorectal cancer, malignant melanoma, papillary thyroid carcinoma, non-small cell lung carcinoma, and adenocarcinoma of lung. More than 30 mutations of the BRAF gene associated with human cancers have been identified. The frequency of BRAF mutations varies widely in human cancers from more than 80% in melanomas, to as little as 0-18% in other tumors, such as 1-3% in lung cancers and 5% in colorectal cancer. In 90% of the cases, a Glu for Val substitution at residue 599 (now referred to as V600E) in the activation segment has been found in human cancers. This mutation has been widely observed in papillary thyroid carcinoma, colorectal cancer and melanomas. Depending on the type of mutation the kinase activity towards MEK may also vary. It has been reported that most of the mutants stimulate enhanced B-RAF kinase activity toward MEK. However, a few mutants act through a different mechanism because although their activity toward MEK is reduced, they adopt a conformation that activates wild-type C-RAF, which then signals to ERK.

KRAS is a protein which in humans is encoded by the KRAS gene. Like other members of the Ras family, the KRAS protein is a GTPase and is an early player in many signal transduction pathways. KRAS is usually tethered to cell membranes because of the presence of an isoprenyl group on its C-terminus. When mutated, KRAS is an oncogene. The protein product of the normal KRAS gene performs an essential function in normal tissue signaling, and the mutation of a KRAS gene is an essential step in the development of many cancers. KRAS acts as a molecular on/off switch, and once it is turned on it recruits and activates proteins necessary for the propagation of growth factor and other receptors' signal, such as c-Raf and PI 3-kinase.

Phosphoinositide 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). They are also known as phosphatidylinositol-3-kinases. The pathway, with oncogene PIK3CA and tumor suppressor PTEN (gene) is implicated in insensitivity of cancer tumors to insulin and IGF1, in calorie restriction. PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110α is mutated in many cancers. Many of these mutations cause the kinase to be more active. The PtdIns(3,4,5)P₃ phosphatase PTEN that antagonises PI 3-kinase signaling is absent from many tumors. Hence, PI 3-kinase activity contributes significantly to cellular transformation and the development of cancer.

AKT protein family, which members are also called protein kinases B (PKB) plays an important role in mammalian cellular signaling. Akt kinase is a serine/threonine kinase which is a downstream effector molecule of phosphoinositide 3-kinase and is involved in protecting a cell from apoptosis. Akt kinase is thought to be involved in the progression of cancer because it stimulates cell proliferation and suppresses apoptosis. Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Since it can block apoptosis, and thereby promote cell survival, Akt1 has been implicated as a major factor in many types of cancer. Akt is known to play a role in the cell cycle. Under various circumstances, activation of Akt was shown to overcome cell cycle arrest in G1 and G2 phases. Moreover, activated Akt may enable proliferation and survival of cells that have sustained a potentially mutagenic impact and, therefore, may contribute to acquisition of mutations in other genes.

Cdk4/cyclin D complexes are involved in phosphorylation of the retinoblastoma protein, which is an essential step in progression of a cell through the G1 phase of the cell cycle. Disruption of Hsp90 activity has been shown to decrease the half life of newly synthesized Cdk4.

Raf-1 is a MAP 3-kinase (MAP3K) which, when activated, can phosphorylate and activate the serine/threonine specific protein kinases ERK1 and ERK2. Activated ERKs play an important role in the control of gene expression involved in the cell division cycle, apoptosis, cell differentiation and cell migration.

The transforming protein of the Rous sarcoma virus, v-src, is a prototype of an oncogene family that induces cellular transformation (i.e., tumorogenesis) by non-regulated kinase activity. Hsp90 has been shown to complex with v-scr and inhibit its degradation.

Hsp90 is required to maintain steroid hormone receptors in conformations capable of binding hormones with high affinity. Inhibition of the action of Hsp90 therefore is expected to be useful in treating hormone-associated malignancies such as breast cancer.

p53 is a tumor suppressor protein that causes cell cycle arrest and apoptosis. Mutation of the p53 gene is found in about half of all human cancers, making it one of the most common genetic alterations found in cancerous cells. In addition, the p53 mutation is associated with a poor prognosis. Wild-type p53 has been shown to interact with Hsp90, but mutated p53 forms a more stable association with Hsp90 than wild-type p53 as a result of its misfolded conformation. A stronger interaction with Hsp90 protects the mutated protein from normal proteolytic degradation and prolongs its half-life. In a cell that is heterozygous for mutated and wild-type p53, inhibition of the stabilizing effect of Hsp90 causes mutant p53 to be degraded and restores the normal transcriptional activity of wild-type p53.

There are two classes of protein kinases (PKs): protein tyrosine kinases (PTKs), which catalyze the phosphorylation of tyrosine kinase residues, and the serine-threonine kinases (STKs), which catalyze the phosphorylation of serine or threonine residues. Growth factor receptors with PTK activity are known as receptor tyrosine kinases. Receptor tyrosine kinases are a family of tightly regulated enzymes, and the aberrant activation of various members of the family is one of the hallmarks of cancer. The receptor tyrosine kinase family can be divided into subgroups that have similar structural organization and sequence similarity within the kinase domain.

The members of the type III group of receptor tyrosine kinases include platelet-derived growth factor receptors (PDGF receptors alpha and beta), colony-stimulating factor receptor (CSF-1R, c-Fms), Fms-like tyrosine kinase (FLT3), and stem cell factor receptor (c-Kit). FLT3 is primarily expressed on immature hematopoietic progenitors and regulates their proliferation and survival.

The FLT3-ITD mutation is also present in about 3% of cases of adult myelodysplastic syndrome and some cases of acute lymphocytic leukemia (ALL). Advani, Current Pharmaceutical Design (2005), 11:3449-3457. FLT3 has been shown to be a client protein of Hsp90, and 17AAG, a benzoquinone ansamycin antibiotic that inhibits Hsp90 activity, has been shown to disrupt the association of FLT3 with Hsp90. The growth of leukemia cells that express either wild type FLT3 or FLT3-ITD mutations was found to be inhibited by treatment with 17AAG. Yao, et al., Clinical Cancer Research (2003), 9:4483-4493.

c-Kit is a membrane type III receptor protein tyrosine kinase which binds Stem Cell Factor (SCF) to its extraellular domain. c-Kit has tyrosine kinase activity and is required for normal hematopoiesis. However, mutations in c-Kit can result in ligand-independent tyrosine kinase activity, autophosphorylation and uncontrolled cell proliferation. Aberrant expression and/or activation of c-Kit have been implicated in a variety of pathologic states. For example, there is evidence of a contribution of c-Kit to neoplastic pathology, including its association with leukemias and mast cell tumors, small cell lung cancer, testicular cancer and some cancers of the gastrointestinal tract and central nervous system. In addition, c-Kit has been implicated in carcinogenesis of the female genital tract, sarcomas of neuroectodermal origin, and Schwann cell neoplasia associated with neurofibromatosis. Yang et al., J Clin Invest. (2003), 112:1851-1861; Viskochil, J Clin Invest. (2003), 112:1791-1793. c-Kit has been shown to be a client protein of Hsp90, and Hsp90 inhibitor 17AAG has been shown to induce apoptosis in Kasumi-1 cells, an acute myeloid leukemia cell line that harbors a mutation in c-Kit.

c-Met is a receptor tyrosine kinase that is encoded by the Met protooncogene and transduces the biological effects of hepatocyte growth factor (HGF), which is also referred to as scatter factor (SF). Jiang, et al., Crit. Rev. Oncol. Hemtol. (1999), 29: 209-248. c-Met and HGF are expressed in numerous tissues, although their expression is normally predominantly confined to cells of epithelial and mesenchymal origin, respectively. c-Met and HGF are required for normal mammalian development and have been shown to be important in cell migration, cell proliferation, cell survival, morphogenic differentiation and the organization of 3-dimensional tubular structures (e.g., renal tubular cells, gland formation, etc.). The c-Met receptor has been shown to be expressed in a number of human cancers. c-Met and its ligand, HGF, have also been shown to be co-expressed at elevated levels in a variety of human cancers, particularly sarcomas. However, because the receptor and ligand are usually expressed by different cell types, c-Met signaling is most commonly regulated by tumor-stroma (tumor-host) interactions. Furthermore, c-Met gene amplification, mutation and rearrangement have been observed in a subset of human cancers. Families with germine mutations that activate c-Met kinase are prone to multiple kidney tumors, as well as tumors in other tissues. Numerous studies have correlated the expression of c-Met and/or HGF/SF with the state of disease progression of different types of cancer, including lung, colon, breast, prostate, liver, pancreas, brain, kidney, ovarian, stomach, skin and bone cancers. Furthermore, the overexpression of c-Met or HGF have been shown to correlate with poor prognosis and disease outcome in a number of major human cancers including lung, liver, gastric and breast.

BCR-ABL is an oncoprotein with tyrosine kinase activity that has been associated with chronic myelogenous leukemia (CML), acute lymphocytic leukemia (ALL) in a subset of patients and acute myelogenous leukemia (AML) in a subset of patients. In fact, the BCR-ABL oncogene has been found in at least 90-95% of patients with CML, about 20% of adults with ALL, about 5% of children with ALL and in about 2% of adults with AML. The BCR-ABL oncoprotein is generated by the translocation of gene sequences from the c-ABL protein tyrosine kinase on chromosome 9 into the BCR sequences on chromosome 22, producing the Philadelphia chromosome. The BCR-ABL gene has been shown to produce at least three alternative chimeric proteins, p230 BCR-ABL, p210 BCR-ABL and p190 BCR-ABL, which have unregulated tyrosine kinase activity. The p210 BCR-ABL fusion protein is most often associated with CML, while the p190 BCR-ABL fusion protein is most often associated with ALL. BCR-ABL has also been associated with a variety of additional hematological malignancies including granulocytic hyperplasia, myelomonocytic leukemia, lymphomas and erythroid leukemia. BCR-ABL fusion proteins exist as complexes with Hsp90 and are rapidly degraded when the action of Hsp90 is inhibited. It has been shown that geldanamycin, a benzoquinone ansamycin antibiotic that disrupts the association of BCR-ABL with Hsp90, results in proteasomal degradation of BCR-ABL and induces apoptosis in BCR-ABL leukemia cells.

Epidermal Growth Factor Receptor (EGFR) is a member of the type 1 subgroup of receptor tyrosine kinase family of growth factor receptors which play critical roles in cellular growth, differentiation and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor α (TGFα), amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands. Gullick, Br. Med. Bull. (1991), 47:87-98; Modijtahedi & Dean, Int. J. Oncol. (1994), 4:277-96; Salomon, et al., Crit. Rev. Oncol. Hematol. (1995), 19:183-232. Aberrant or overexpression of EGFR has been associated with an adverse prognosis in a number of human cancers, including head and neck, breast, colon, prostate, lung (e.g., NSCLC, adenocarcinoma and squamous lung cancer), ovarian, gastrointestinal cancers (gastric, colon, pancreatic), renal cell cancer, bladder cancer, glioma, gynecological carcinomas and prostate cancer. In some instances, overexpression of tumor EGFR has been correlated with both chemoresistance and a poor prognosis. Lei, et al., Anti-cancer Res. (1999),19:221-28; Veale, et al., Br. J. Cancer (1993); 68:162-65. Mutations in EGFR are associated with many types of cancer as well. For example, EGFR mutations are highly prevalent in non-mucinous BAC patients. Finberg, et al., J. Mol. Diagnostics (2007) 9(3):320-26.

Hsp90 has been shown by mutational analysis to be necessary for the survival of normal eukaryotic cells. However, Hsp90 is over expressed in many tumor types indicating that it may play a significant role in the survival of cancer cells, and that cancer cells may be more sensitive to inhibition of Hsp90 than normal cells. For example, cancer cells typically have a large number of mutated and overexpressed oncoproteins that are dependent on Hsp90 for folding. In addition, because the environment of a tumor is typically hostile due to hypoxia, nutrient deprivation, acidosis, etc., tumor cells may be especially dependent on Hsp90 for survival. Moreover, inhibition of Hsp90 causes the simultaneous inhibition of a number of oncoproteins, hormone receptors and transcription factors, thus making it an attractive target for an anti-cancer agent. In fact, benzoquinone ansamycins, a family of natural products that inhibit Hsp90, have shown evidence of therapeutic activity in clinical trials.

Although initially promising, first generation Hsp90 inhibitors, the benzoquinone ansamycins, and their derivatives, suffer from some limitations. For example, they have low oral bioavailability and their limited solubility makes them difficult to formulate. In addition, they are metabolized by polymorphic cytochrome P450 CYP3A4 and are a substrate for the P-glycoprotein export pump involved in the development of multidrug resistance. Additionally, the ansamycin class of Hsp90 inhibitors has shown serious toxicity problems. Therefore, a need exists for new therapeutics that improve the prognosis of cancer patients and that reduce or overcome the limitations of currently used anti-cancer agents.

Despite the availability of multiple therapeutic regimens to treat proliferative disorders such as cancer, many patients do not respond to any treatments. Of those that do respond to standard therapies, the effect is usually short-lived as resistance develops to the initial therapeutic regimens. There is an immediate need in the art for improvement in cancer therapies, both in terms of the proportion of patients who respond to therapy and the survival benefit imparted.

### SUMMARY OF THE INVENTION

It has been found that certain triazolone Hsp90 inhibitors are surprisingly effective at treating subjects with metastatic or unresectable solid tumors with a tolerable side effect profile. While efficacy of a compound at treating the target diseases may be essential, it is also essential to be able to administer the drug to a subject in need thereof. With poorly water soluble compounds, such as the compounds according to formulae (I)-(IV), and in Table 1, the creation of a drug product for administration to a subject can be a substantial hurdle. Intravenous (IV) formulations have to be simultaneously able to dissolve the product, both in the vial and in the infusion bag, and be made of solvents, surfactants and excipients that are pharmaceutically acceptable in total doses that are non-toxic for the subject.

The present invention provides a pharmaceutical composition (formulation) comprising an Hsp90 inhibitor according to formulae (I)-(IV), or a compound shown in Table 1. The invention also provides a dosing regimen for the pharmaceutical composition and a method of administering the pharmaceutical composition to a subject in need thereof.

In one embodiment, the pharmaceutical composition comprises a pharmaceutically acceptable organic solvent such as polyethylene glycol (PEG), dimethyl sulfoxide (DMSO), N-methylpyrolidinone (NMP), or glycerine, a pharmaceutically acceptable surfactant such as polysorbate 80, cremophor, or polyvinyl povidone (PVP), and a compound according to formulae (I)-(IV), or as detailed in Table 1. In one embodiment, the pharmaceutical composition further comprises a pharmaceutically acceptable co-solvent such as propylene glycol or dehydrated alcohol (ethanol). In one embodiment, the pharmaceutically acceptable organic solvent is a polyethylene glycol (PEG) weighing from about 200 daltons to about 450 daltons. In one embodiment, the pharmaceutically acceptable organic solvent is PEG-300. In one embodiment, the pharmaceutically acceptable surfactant is polysorbate 80. In one embodiment, the pharmaceutical composition further comprises a co-solvent which is a pharmaceutically acceptable alcohol. In one embodiment, the co-solvent is dehydrated alcohol (ethanol).

In one embodiment, the pharmaceutical composition comprises a pharmaceutically acceptable organic solvent, a pharmaceutically acceptable surfactant and a compound according to formulae (I)-(IV) or in Table 1, wherein the v/v ratio of organic solvent to surfactant is about 9:1. In one embodiment, the pharmaceutical composition comprises about 90% v/v PEG-300, about 10% v/v polysorbate 80, and compound 1. In one aspect of this embodiment, compound 1 is present in the pharmaceutical composition at a concentration of about 8 mg/mL.

In one embodiment, the pharmaceutical composition comprises a pharmaceutically acceptable organic solvent, pharmaceutically acceptable surfactant, a pharmaceutically acceptable co-solvent and a compound according to formulae (I)-(IV) or a compound in Table 1. In one embodiment, the pharmaceutical composition contains a v/v/v ratio of organic solvent to surfactant to co-solvent of about 39.35:35:25. In one embodiment, the pharmaceutical composition comprises about 39.35% v/v PEG-300, about 35% v/v polysorbate 80, about 25% v/v dehydrated alcohol, and compound 1. In one aspect of this embodiment, compound 1 is present in the pharmaceutical composition at a concentration of about 25 mg/mL.

In one embodiment, the pharmaceutical composition contains a v/v/v ratio of organic solvent to surfactant to co-solvent of about 37.5:37.5:25. In one embodiment, the pharmaceutical composition comprises about 37.5% v/v PEG-300, about 37.5% v/v polysorbate 80, about 25% v/v dehydrated alcohol, and compound 1. In one aspect of this embodiment, compound 1 is present in the pharmaceutical composition at a concentration of about 20 mg/mL.

In one embodiment, the pharmaceutical composition contains a v/v/v ratio of organic solvent to surfactant to co-solvent of about 55:25:20. In one embodiment, the pharmaceutical composition comprises about 55% v/v PEG-300, about 25% v/v polysorbate 80, about 20% v/v dehydrated alcohol, and compound 1. In one aspect of this embodiment, compound 1 is present in the pharmaceutical composition at a concentration of about 20 mg/mL.

In one embodiment, the invention includes a method of administering the pharmaceutical composition described herein to a subject in need thereof using a silicone catheter. In one embodiment, the silicone catheter is an in-dwelling catheter. In one embodiment, the pharmaceutical composition is administered to the subject via peripheral venous access. In one embodiment, the pharmaceutical composition is administered to the subject via peripherally inserted central catheter. In one embodiment, the pharmaceutical composition is administered intravenously. In one embodiment, the invention also includes a kit for administering a pharmaceutical composition comprising a silicone catheter and one or more vials of the pharmaceutical composition described herein.

In one embodiment, the method includes treating a subject in need thereof comprising administering a pharmaceutical composition described herein at a dose of about 75 mg/m² to about 150 mg/m² of compound 1, approximately twice a week. In one embodiment, the dose of compound 1 is about 100 mg/m² to about 125 mg/m², administered twice a week. In one embodiment, the dose of compound 1 is about 120 mg/m², administered twice a week. In one embodiment, the dose of compound 1 is about 100 mg/m², administered twice a week.

In one embodiment, the method includes treating a subject in need thereof comprising administering a pharmaceutical composition described herein at a dose of about 120 mg/m² to about 250 mg/m² of compound 1, approximately once a week. In one embodiment, the dose of compound 1 is about 150 mg/m² to about 215 mg/m², administered once a week. In one embodiment, the dose of compound 1 is about 175 mg/m² to about 200 mg/m², administered once a week. In one embodiment, the dose of compound 1 is about 200 mg/m², administered once a week.

In one embodiment, the pharmaceutical composition comprises about 39.35% v/v PEG-300, about 35% v/v polysorbate 80, about 25% v/v dehydrated alcohol, and compound 1 at a concentration of about 25 mg/mL. In one aspect of this embodiment, the pharmaceutical composition is administered to a subject in need thereof once a week at a dose of about 200 mg/m². In one aspect of this embodiment, the pharmaceutical composition is administered to a subject in need thereof twice a week at a dose of about 120 mg/m². In any of these embodiments, the pharmaceutical composition is administered via a silicone in-dwelling catheter, or by peripheral venous access. In one embodiment, the pharmaceutical composition is administered to the subject via a silicone in-dwelling catheter.

In one embodiment, the pharmaceutical composition is administered as a single agent. In another embodiment, the pharmaceutical composition is administered in combination with one or more pharmaceutical compositions containing additional therapeutic agent(s). In any one of these embodiments, the pharmaceutical composition contains a compound represented in Table 1. In one embodiment, the pharmaceutical composition is administered to a subject with a solid tumor. In one embodiment, the pharmaceutical composition is administered to a subject with a hematological malignancy. In one embodiment, the subject has a solid tumor with mutations or translocations in EGFR, K-ras, HER2neu, B-raf, PI3K and/or ALK proteins. In one embodiment, the subject has a solid tumor with wild type EGFR and K-ras. In one embodiment, the subject has a solid tumor with mutations in EGFR and wild type K-ras. In one embodiment, subject has a tumor with wild type EGFR and mutations in the K-ras protein. In one embodiment, the subject has a tumor with an ALK-elm4 translocation. In one embodiment, the subject has a tumor with a HER2neu mutation. In one embodiment, the subject has a tumor with a mutation in PI3K. In one embodiment, the subject has tumor with a mutation in the B-raf protein.

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker Alkoxy groups included in compounds described herein may be optionally substituted with one or more substituents.

As used herein, a “haloalkoxy” is a haloalkyl group which is attached to another moiety via an oxygen linker.

As used herein, the term “aryl” means a mono- or polycyclic hydrocarbon, containing from 6 to 15 carbon atoms, in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. A preferred aryl group is a phenyl. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³⁰ or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C1-C4 alkyl, C1-C4 haloalkyl, C1-C4 alkoxy, C1-C4 haloalkoxy, C1-C4 hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “compound(s) described herein” and similar terms refers to a compound of formulae (I), (II), (III) or (IV) or a compound in Table 1 or a tautomer or pharmaceutically acceptable salt thereof. Also included in the scope of the present invention are the anhydrous form of the compound, a solvate, clathrate, non-clathrates, hydrate or polymorph of a compound of formulae (I), (II), (III), or (IV), or a compound in Table 1.

When a disclosed compound is named or depicted by structure, it is to be understood that solvates (e.g., hydrates) of the compound or a pharmaceutically acceptable salt thereof is also included as well as the anhydrous form. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water. The anhydrous form means that substantially no water or solvent is incorporated into the crystal lattice during crystallization.

When a disclosed compound is named or depicted by structure, it is to be understood that clathrates (“inclusion compounds”) of the compound or its pharmaceutically acceptable salt, solvate or polymorph, are also included, as well as compounds that are not clathrates. “Clathrate” means a compound described herein, or a salt thereof, in the form of a crystal lattice that contains spaces (e.g., channels) that have a guest molecule trapped within (e.g., a solvent or water).

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

Some of the disclosed pharmaceutical compositions can be particularly effective at treating subjects with proliferative disorders. In one embodiment, the proliferative disorder is cancer. In one embodiment, the pharmaceutical composition is administered to a subject whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

Other anti-proliferative or anti-cancer therapies may be combined with the compounds described herein to treat proliferative diseases and cancer. Other therapies or anti-cancer agents that may be used in combination with the inventive anti-cancer agents described herein include surgery, radiotherapy (including, but not limited to, gamma-radiation, neutron beam radiotherapy, electron beam radiotherapy, proton therapy, brachytherapy, and systemic radioactive isotopes), endocrine therapy, biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs.

As used herein, the term “pharmaceutically acceptable salt” refers to a salt prepared from a compound of formulae (I)-(IV) or a compound in Table 1 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I)-(IV) or a compound in Table 1 having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

A pharmaceutically acceptable carrier may contain inert ingredients which do not unduly inhibit the biological activity of the compound(s). The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound described herein being used. In cases where no amount is expressly noted, an effective amount should be assumed. Non-limiting examples of an effective amount of a compound described herein are provided below.

In one embodiment, the pharmaceutical composition is formulated to deliver a dose of about 50 mg/m², about 75 mg/m², about 100 mg/m², about 125 mg/m², about 150 mg/m², about 175 mg/m², about 200 mg/m², about 225 mg/m², or about 250 mg/m². Administration of a compound described herein may occur once a week or twice a week. In one embodiment, the pharmaceutical composition is formulated at a dose of about 200 mg/m² and administered once a week. Other dosing regimens are discl

In one embodiment, the pharmaceutical composition is administered parentally. In one embodiment, the pharmaceutical composition is administered intravenously through an in-dwelling port or through peripheral access. In one embodiment, the pharmaceutical composition is administered through a silicone catheter in an in-dwelling port.

In one embodiment, the pharmaceutical compositions described herein are administered once or twice every week for three out of four weeks, with the fourth week being a “rest week” for the subject being treated. In one embodiment, the pharmaceutical compositions described herein can also be administered once or twice a week for more than three consecutive weeks, with no rest week.

“Mutations” are changes in the DNA sequence of a cell's genome and are caused by radiation, viruses, transposons and mutagenic chemicals, as well as errors that occur during meiosis or DNA replication. They can include point mutations, insertions or deletions. None mutated DNA sequences are classified as “wild type”.

A “translocation” occurs when a portion of one chromosome is transferred to another chromosome. There are two main types of translocations. In a reciprocal translocation, segments from two different chromosomes have been exchanged. In a Robertsonian translocation, an entire chromosome has attached to another at the Centromere—in humans these only occur with chromosomes 13, 14, 15, 21 and 22.

As used herein, the terms “treat”, “treatment” and “treating” refer to the reduction or amelioration of the progression, severity and/or duration of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which said therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

The compounds described herein are defined by their chemical structures and/or chemical names. Where a compound is referred to by both a chemical structure and a chemical name, and the chemical structure and chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

The present invention utilizes compounds having Formulae (I), (II), (III) and (IV), and those set forth in Table 1 and tautomers or pharmaceutically acceptable salts thereof.

In one aspect, the invention provides a method to treat cancer in a subject in need thereof, comprising administering an effective amount of an Hsp90 inhibitor according to formula (I) as set forth below:

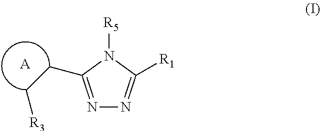

or a tautomer, or pharmaceutically acceptable salts thereof, wherein:

ring A is an aryl or a heteroaryl, wherein the aryl or the heteroaryl are optionally further substituted with one or more substituents in addition to R₃.

R₁ and R₃ are, independently, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂.

R₅ is 1) an optionally substituted heteroaryl or an optionally substituted 8 to 14 membered aryl; 2) a substituted phenyl, wherein the phenyl group is substituted with: i) one substituent selected from nitro, cyano, a haloalkoxy, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxylalkyl, alkoxyalkyl, guanadino, —NR₁₀R₁₁, —OR₇, —C(O)R₇, —C(O)OR₇, —OC(O)R₇, —C(O)NR₁₀R₁₁, —NR₈C(O)R₇, —S(O)ₚR₇, —OS(O)ₚR₇, —S(O)ₚOR₇, —NR₈S(O)ₚR₇, —S(O)ₚNR₁₀R₁₁, —S(O)ₚOR₇, —OP(O)(OR₇)₂, or —SP(O)(OR₇)₂; or ii) two to five substituents selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, —F, —Br, —I, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, —OP(O)(OR₇)₂, or —SP(O)(OR₇)₂, —S(O)ₚOR₇; or 3) an optionally substituted cycloalkyl, and optionally substituted cycloalkenyl, or a substituted alkyl, wherein the alkyl group is substituted with one or more substituents independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁, —S(O)ₚOR₇, —OP(O)(OR₇)₂, or —SP(O)(OR₇)₂.

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl.

R₂₆ is a C1-C6 alkyl;

p, for each occurrence, is, independently, 0, 1 or 2; and

m, for each occurrence, is independently, 1, 2, 3, or 4.

In one embodiment of the method, the Hsp90 inhibitor is according to formula (II):

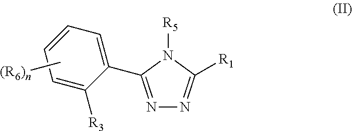

or a tautomer, or a pharmaceutically acceptable salt thereof, wherein R₆, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, [[or]] —SP(O)(OR₇)₂, —NR₇C(O)R₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —NR₇S(O)ₚR₇, —C(NR₈)OR₇, or —S(O)ₚR₇; and n is zero of an integer from 1 to 4. The values and particular values of the rest of the variables are as described for formula (I).

In another embodiment of the method, the Hsp90 inhibitor is according to formula (III):

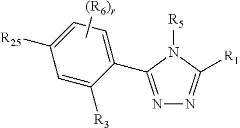

or a tautomer, or a pharmaceutically acceptable salt thereof, wherein R₂₅ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂; and r is zero or an integer from 1 to 3. The values and particular values of the rest of the variables are as described for formula (I) or (II).

In another embodiment of the method, the Hsp90 inhibitor is according to formula (IV):

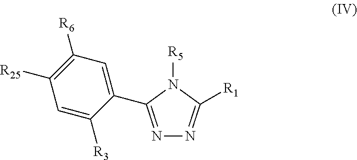

or a tautomer, or a pharmaceutically acceptable salt thereof. The values and particular values of the rest of the variables are as described for formula (I), (II) or (III).

In one embodiment, the Hsp90 inhibitor is a compound according to formula (I), (II), (III) or (IV), wherein R₁ and R₃ are each independently —OH, —SH, —NHR₇, —OC(O)NR₁₀R₁₁, —SC(O)NR₁₀R₁₁, —OC(O)R₇, —SC(O)R₇, —OC(O)OR₇, —SC(O)OR₇, —OS(O)ₚR₇, —S(O)ₚOR₇, —SS(O)ₚR₇, —OS(O)ₚOR₇, —SS(O)ₚOR₇, —OC(S)R₇, —SC(S)R₇, —OC(S)OR₇, —SC(S)OR₇, —OC(S)NR₁₀R₁₁, —SC(S)NR₁₀R₁₁, —OC(NR₈)R₇, —SC(NR₈)R₇, —OC(NR₈)OR₇, —SC(NR₈)OR₇, —OP(O)(OR₇)₂ or —SP(O)(OR₇)₂. In one embodiment, the Hsp90 inhibitor is a compound according to formula (I), (II), (III) or (IV), wherein R₁ and R₃ are each, independently, —OH, —SH, or —NHR₇. In another embodiment, the Hsp90 inhibitor is of a compound according to formula (I), (II), (III) or (IV), wherein R₁ and R₃ are both —OH. Wherein the values and particular values of the rest of the variables are as described above.

In one embodiment of the method, the Hsp90 inhibitor is a compound according to formula (III) or (IV), wherein R₂₅ is —OH or —OR₇. In another embodiment, the compound is according to formula (III) or (IV), wherein R₂₅ is —OH. Wherein the values and particular values of the rest of the variables are as described above.

In one embodiment, the Hsp90 inhibitor is a compound according to formula (II), (III) or (IV), wherein R₆ is a C1-C6 alkyl, a C1-C6 haloalkyl, a C1-C6 alkoxy, a C1-C6 haloalkoxy, a C1-C6 alkyl sulfanyl or a C3-C6 cycloalkyl. In another embodiment, R₆ is a C1-C5 alkyl, or a C3-C6 cycloalkyl. In another embodiment, R₆ is ethyl, isopropyl, n-propyl, n-butyl, isobutyl, or cyclopropyl. In another embodiment, R₆ is isopropyl. Wherein the values and particular values of the rest of the variables are as described above.

In one embodiment, the Hsp90 inhibitor is a compound according to formula (I), (II), (III) or (IV), wherein R⁵ is a C1-C4 alkyl or a C3-C6 cycloalkyl, each optionally substituted with a C3-C6 cycloalkyl, halo, cyano, nitro, C1-C4 haloalkyl, —NR₁₀R₁₁, —OR₇, —C(O)R₇, —C(O)NR₁₀R₁₁, —NR₈C(O)R₇, —SR₇, —S(O)ₚR₇, —NR₈S(O)ₚR₇, or —S(O)ₚNR₁₀R₁₁. In another embodiment, the Hsp90 inhibitor is a compound according to formula (I), (II), (III) or (IV), wherein R₅ is a phenyl, substituted with 1) one or more substituents selected from the group consisting of phenyl, 5-6 membered heteroaryl, (C1-C3)alkoxy-(C1-C4)alkyl, —OR₇, —NR₁₀R₁₁, —COR₇, —C(O)OR₇, —C(O)NR₁₀R₁₁, and —S(O)ₚR₇; or 2) two or three substituents selected from the group consisting of C1-C4 alkyl, halo, C1-C4 haloalkyl, phenyl, 5-6 membered heteroaryl, (C1-C3)alkoxy-(C1-C4)alkyl, —OR₇, —NR₁₀R₁₁, —COR₇, —C(O)OR₇, —C(O)NR₁₀R₁₁, and —S(O)ₚR₇. In another embodiment, the Hsp90 inhibitor is a compound according to formula (I), (II), (III) or (IV), wherein R₅ is a phenyl substituted with: 1) one or more phenyl, tetrazole, C1-C4 alkoxy, C1-C4 thioalkoxy, —N(R₁₂)₂, or —C(O)OR₁₂; or 2) two or three C1-C4 alkyl, halo, phenyl, tetrazole, C1-C4 alkoxy, C1-C4 thioalkoxy, —N(R₁₂)₂, or —C(O)OR₁₂; and each R₁₂ is independently —H or C1-C4 alkyl. In another embodiment, the Hsp90 inhibitor is a compound according to formula (I), (II), (III) or (IV), wherein R₅ is a bicyclic or tricyclic heteroaryl or bicyclic aryl, each optionally and independently substituted with one or more C1-C4 alkyl, C3-C6 cycloalkyl, halo, cyano, nitro, C1-C4 haloalkyl, C1-C4 hydroxyalkyl, (C1-C3)alkoxy-(C1-C4)alkyl, —NR₁₀R₁₁, —OR₇, —C(O)R₇, —C(O)NR₁₀R₁₁, —NR₈C(O)R₇, —SR₇, —S(O)ₚR₇, —NR₈S(O)ₚR₇, or —S(O)ₚNR₁₀R₁₁. In another embodiment, the Hsp90 inhibitor is a compound according to formula (I), (II), (III) or (IV), wherein R₅ is represented by:

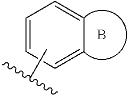

wherein [symbol] represents the point of attachment to the triazole ring of formula (I)-(IV); ring B is 1) a 5-6 membered heterocyclyl or a 5-6 membered heteroaryl, each optionally fused to a (C5-C6) cycloalkyl or phenyl; or 2) a phenyl or C5-C6 cycloalkyl; and ring B is optionally substituted with one or more C1-C6 alkyl, C3-C6 cycloalkyl, 5-7 membered heterocyclyl, phenyl, 5-7 membered heteroaryl, halo, cyano, nitro, C1-C4 haloalkyl, C1-C4 hydroxyalkyl, (C1-C3)alkoxy-(C1-C4)alkyl, —NR₁₀R₁₁, —OR₇, —C(O)R₇, —C(O)OR₇, —C(O)NR₁₀R₁₁, —NR₈C(O)R₇, —S(O)ₚR₇, —NR₈S(O)ₚR₇, or —S(O)ₚNR₁₀R₁₁. In another embodiment, the Hsp90 inhibitor is a compound according to formula (I), (II), (III) or (IV), wherein R₅ is a naphthyl, quinolinyl, 1,2,3,4-tetrahydronaphthyl, 2,3-dihydro-1H-indenyl or a moiety represented by:

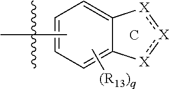

wherein [symbol] represents the point of attachment to the triazole ring of formula (I)-(IV); [symbol] in ring C represents a possible position for a double bond; ring C can be either aromatic or non-aromatic; each X is independently ═C(R₇)—, —C(R₇)₂—, —C(O)—, —N(R₇)—, —N═, or O; R⁵ can be substituted with one or more R₁₃ moiety; each R₁₃ is independently C1-C6 alkyl, C3-C6 cycloalkyl, 5-7 membered heterocyclyl, phenyl, 5-7 membered heteroaryl, halo, cyano, nitro, C1-C4 haloalkyl, C1-C4 hydroxyalkyl, (C1-C3)alkoxy-(C1-C4)alkyl, —NR₁₀R₁₁, —OR₇, —C(O)R₇, —C(O)OR₇, —C(O)NR₁₀R₁₁, —NR₈C(O)R₇, —S(O)ₚR₇, —NR₈S(O)ₚR₇, or —S(O)ₚNR₁₀R₁₁; and q is 0, 1, 2 or 3. In any one of the embodiments describing in this paragraph, the values and particular values of the rest of the variables are as described in any one of the preceding paragraphs.

In another embodiment, the Hsp90 inhibitor is a compound according to formula (I), (II), (III) or (IV), wherein R₅ is represented by:

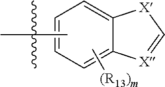

wherein X′ is independently —NR₇ or O; X″ is —N═ or —CR₇—; and one or more R₁₃ moiety can be located on either ring, each R₁₃ is independently C1-C6 alkyl, C3-C6 cycloalkyl, 5-7 membered heterocyclyl, phenyl, 5-7 membered heteroaryl, halo, cyano, nitro, C1-C4 haloalkyl, C1-C4 hydroxyalkyl, (C1-C3)alkoxy-(C1-C4)alkyl, —NR₁₀R₁₁, —OR₇, —C(O)R₇, —C(O)OR₇, —C(O)NR₁₀R₁₁, —NR₈C(O)R₇, —S(O)ₚR₇, —NR₈S(O)ₚR₇, or —S(O)ₚNR₁₀R₁₁; and q is 0, 1, 2 or 3. In one aspect of this embodiment, X′ is NR₇ and X″ is CR₇; each R₇ is independently H or C1-C4 alkyl; each R₁₃ is independently C1-C4 alkyl, —OR₁₂, C1-C4 haloalkyl, C1-C4 hydroxyalkyl, (C1-C3)alkoxy-(C1-C4)alkyl, —C(O)OR₁₂, or —C(O)NR₁₂R₁₂; and m is 0, 1 or 2. In another aspect of this embodiment, X′ is NR₇ and X″ is CH; R₇ is methyl; and m is 0. In another aspect of this embodiment, X′ is O and X″ is N or CR₇. In any aspect of the embodiment described in this paragraph, the values and particular values of R₁, R₃, R₆, R₇, R₈, R₁₀, R₁₁, R₂₅, R₂₆, n, m, p, q and r are as described in the preceding paragraphs.

In one aspect, the Hsp90 inhibitor is a compound according to formula (I), (II), (III) or (IV), R₅ is

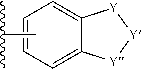

wherein Y is —O— or —NR₁₂—; Y′ is —C(O)—, or —C(R₁₂)₂—; Y″ is —O—, —NR₁₂— or —C(R₁₂)₂—; and each R₁₂ is independently H or C1-C3 alkyl. In one aspect of this embodiment, Y is O; Y′ is C(R₁₂)₂; Y″ is O; and each R₁₂ is H. In another aspect of this embodiment, Y is NR₁₂, Y′ is C(O), Y″ is NR₁₂; and each R₁₂ is H. In another aspect of this embodiment, Y is NR₁₂, Y′ is C(R₁₂)₂, Y″ is NR₁₂; and each R₁₂ is H or C1-C3 alkyl. In any aspect of the embodiment described in this paragraph, the values and particular values of R₁, R₃, R₆, R₇, R₈, R₁₀, R₁₁, R₂₅, R₂₆, n, m, p, q and r are as described in the preceding paragraphs.

In one embodiment of the method, the Hsp90 inhibitor is a compound according to formula (I), (II), (III) or (IV), wherein R₅ is naphthyl-1-yl, N-methyl-indol-5-yl, N-isopropyl-indol-5-yl, 1,3-dimethyl-indol-5-yl, 1,2-dimethyl-indol-5-yl, 1-isopropyl-7-methoxy-indol-5-yl, or 2,3-dimethyl-indol-5-yl. In one embodiment, the Hsp90 inhibitor is a compound according to formula (I), (II), (III) or (IV), wherein R₅ is N-methyl-indol-5-yl. In any embodiment described in this paragraph, the values and particular values of R₁, R₃, R₆, R₇, R₈, R₁₀, R₁₁, R₂₅, R₂₆, n, m, p, q and r are as described in the preceding paragraphs.

In one embodiment of the method, the Hsp90 inhibitor is selected from: 3-(2-Hydroxyphenyl)-4-(naphthalen-1-yl)-5-mercapto-[1,2,4]triazole; 3-(2,4-Dihydroxyphenyl)-4-[4-(2-methoxyethoxy)-naphthalen-1-yl]-5-mercapto-[1,2,4]triazole; 3-(2,4-Dihydroxyphenyl)-4-(2-methyl-4-bromophenyl)-5-mercapto-[1,2,4]triazole; 3-(2,4-Dihydroxyphenyl)-4-(4-bromophenyl)-5-mercapto-[1,2,4]triazole; 3-(3,4-Dihydroxyphenyl)-4-(6-methoxy-naphthalen-1-yl)-5-mercapto-[1,2,4]triazole; 3-(3,4-Dihydroxyphenyl)-4-(6-ethoxy-naphthalen-1-yl)-5-mercapto-[1,2,4]triazole; 3-(3,4-Dihydroxyphenyl)-4-(6-propoxy-naphthalen-1-yl)-5-mercapto-[1,2,4]triazole; 3-(2,4-Dihydroxy-5-ethyl-phenyl)-4-(5-methoxy-naphthalen-1-yl)-5-mercapto-[1,2,4]triazole; 3-(3,4-Dihydroxyphenyl)-4-(6-isopropoxy-naphthalen-1-yl)-5-mercapto-[1,2,4]triazole; 3-(2,4-Dihydroxyphenyl)-4-(2,6-diethylphenyl)-5-mercapto-[1,2,4]triazole; 3-(2,4-Dihydroxyphenyl)-4-(2-methy-6-ethylphenyl)-5-mercapto-[1,2,4]triazole; 3-(2,4-Dihydroxyphenyl)-4-(2,6-diisopropylphenyl)-5-mercapto-[1,2,4]triazole; 3-(2,4-Dihydroxyphenyl)-4-(1-ethyl-indol-4-yl)-5-mercapto-[1,2,4]triazole; 3-(2,4-Dihydroxyphenyl)-4-(2,3-dihydro-benzo[1,4]dioxin-5-yl)-5-mercapto-[1,2,4]triazole; 3-(2,4-Dihydroxyphenyl)-4-(3-methylphenyl)-5-mercapto-[1,2,4]triazole; 3-(2,4-Dihydroxyphenyl)-4-(4-methylphenyl)-5-mercapto-[1,2,4]triazole; 3-(2,4-Dihydroxyphenyl)-4-(2-chlorophenyl)-5-mercapto-[1,2,4]triazole; 3-(2,4-Dihydroxyphenyl)-4-(3-chlorophenyl)-5-mercapto-[1,2,4]triazole; 3-(2,4-Dihydroxyphenyl)-4-(4-chlorophenyl)-5-mercapto-[1,2,4]triazole; 3-(2,4-Dihydroxyphenyl)-4-(2-methoxyphenyl)-5-mercapto-[1,2,4]triazole; 3-(2,4-Dihydroxyphenyl)-4-(3-methoxyphenyl)-5-mercapto-[1,2,4]triazole; 3-(2,4-Dihydroxyphenyl)-4-(4-methoxyphenyl)-5-mercapto-[1,2,4]triazole; 3-(2,4-Dihydroxyphenyl)-4-(3-fluorophenyl)-5-mercapto-[1,2,4]triazole; 3-(2,4-Dihydroxyphenyl)-4-(2-ethylphenyl)-5-mercapto-[1,2,4]triazole; 3-(2-Hydroxy-4-fluorophenyl)-4-(naphthalen-1-yl)-5-mercapto-[1,2,4]triazole; 3-(2-Hydroxy-4-aminophenyl)-4-(naphthalen-1-yl)-5-mercapto-[1,2,4]triazole; 3-(2,4-Dihydroxyphenyl)-4-(2-methyl-4-butyl-phenyl)-5-mercapto-[1,2,4]triazole; 3-(2,4-Dihydroxyphenyl)-4-(2,4-dimethyl-phenyl)-5-mercapto-[1,2,4]triazole; 3-(2,4-Dihydroxyphenyl)-4-(2,6-dimethyl-phenyl)-5-mercapto-[1,2,4]triazole; 3-(2,4-Dihydroxyphenyl)-4-(2,6-dimethyl-phenyl)-5-mercapto-[1,2,4]triazole; 3-(2,4-Dihydroxyphenyl)-4-(4-fluorophenyl)-5-mercapto-[1,2,4]triazole; 3-(2,4-Dihydroxyphenyl)-4-(2-methylsulfanylphenyl)-5-mercapto-[1,2,4]triazole; 3-(2,4-Dihydroxyphenyl)-4-(naphthalene-2-yl)-5-mercapto-[1,2,4]triazole; 3-(2,4-Dihydroxyphenyl)-4-(2,3-dimethylphenyl)-5-mercapto-[1,2,4]triazole; 3-(2,4-Dihydroxyphenyl)-4-(2-methyl-4-fluorophenyl)-5-mercapto-[1,2,4]triazole; 3-(2,4-Dihydroxyphenyl)-4-(acenaphthalen-5-yl)-5-mercapto-[1,2,4]triazole; 3-(2-Hydroxy-4-methoxy-phenyl)-4-(naphthalen-1-yl)-5-mercapto-[1,2,4]triazole; 3-(2,4-Dihydroxyphenyl)-4-(2,3-dichlorophenyl)-5-mercapto-[1,2,4]triazole; 3-(2,4-Dihydroxyphenyl)-4-(5-methoxynaphthalen-1-yl)-5-mercapto-[1,2,4]triazole; 3-(2,4-Dihydroxyphenyl)-4-(pyren-1-yl)-5-mercapto-[1,2,4]triazole; 3-(2,4-Dihydroxyphenyl)-4-(quinolin-5-yl)-5-mercapto-[1,2,4]triazole; 3-(2,4-Dihydroxyphenyl)-4-(1,2,3,4-tetrahydronaphthalen-5-yl)-5-mercapto-[1,2,4]triazole; 3-(2,4-Dihydroxyphenyl)-4-(anthracen-1-yl)-5-mercapto-[1,2,4]triazole; 3-(2,4-Dihydroxyphenyl)-4-(biphenyl-2-yl)-5-mercapto-[1,2,4]triazole; 3-(2,4-Dihydroxy-6-methyl-phenyl)-4-(naphthalene-1-yl)-5-mercapto-[1,2,4]triazole; 3-(2,4-Dihydroxyphenyl)-4-(4-pentyloxyphenyl)-5-mercapto-[1,2,4]triazole; 3-(2,4-Dihydroxyphenyl)-4-(4-octyloxyphenyl)-5-mercapto-[1,2,4]triazole; 3-(2,4-Dihydroxyphenyl)-4-(4-chloronaphthalen-1-yl)-5-mercapto-[1,2,4]triazole; 3-(2,4-Dihydroxy-5-ethyl-phenyl)-4-(naphthalen-1-yl)-5-mercapto-[1,2,4]triazole; 3-(2,4-Dihydroxy-5-ethyl-phenyl)-4-(7-carboxymethoxy-naphthalen-1-yl)-5-mercapto-[1,2,4]triazole; 3-(2,4-Dihydroxyphenyl)-4-(2-methyl-quinolin-4-yl)-5-mercapto-[1,2,4]triazole; 3-(3-Hydroxypyridin-4-yl)-4-(naphthalen-1-yl)-5-mercapto-[1,2,4]triazole; 3-(2-Hydroxy-4-acetylamino-phenyl)-4-(naphthalen-1-yl)-5-mercapto-[1,2,4]triazole; 3-(2,4-Dihydroxy-phenyl)-4-(1,2,3,4-tetrahydronaphthalen-1-yl)-5-mercapto-[1,2,4]triazole; 3-(2,4-Dihydroxy-phenyl)-4-(2,3-dihydro-benzo[1,4]dioxin-5-yl)-5-mercapto-[1,2,4]triazole; 3-(2,4-Dihydroxy-phenyl)-4-(3,5-dimethoxyphenyl)-5-mercapto-[1,2,4]triazole; 3-(2,4-Dihydroxy-phenyl)-4-(2,3-dimethyl-1H-indol-4-yl)-5-mercapto-[1,2,4]triazole; 3-(2,4-Dihydroxy-3-propyl-phenyl)-4-(naphthalen-1-yl)-5-mercapto-[1,2,4]triazole; 3-(1-ethyl-4-hydroxy-6-oxo-1,6-dihydro-pyridin-3-yl)-4-(naphthalen-1-yl)-5-mercapto-[1,2,4]triazole; 3-(4-hydroxy-6-oxo-pyridin-3-yl)-4-(naphthalen-1-yl)-5-mercapto-[1,2,4]triazole; 3-(2,4-Dihydroxy-phenyl)-4-(3,5-di-tert-butylphenyl)-5-mercapto-[1,2,4]triazole; 3-(2,6-Dihydroxy5-fluoro-pyridin-3-yl) 4-(naphthalen-1-yl)-5-mercapto-[1,2,4]triazole; 3-(2,4-Dihydroxy-5-methyl-phenyl)-4-(naphthalene-1-yl)-5-mercapto-[1,2,4]triazole; 3-(2,4-Dihydroxy-phenyl]-4-(3-benzoylphenyl)-5-mercapto-[1,2,4]triazole; 3-(2,4-Dihydroxy-phenyl)-4-(4-carboxy-naphthalen-1-yl)-5-mercapto-[1,2,4]triazole; 3-(2,4-Dihydroxy-phenyl)-4-[4-(N,N-dimethylcarbamoyl)-naphthalen-1-yl]-5-mercapto-[1,2,4]triazole; 3-(2,4-Dihydroxy-phenyl)-4-(4-propoxy-naphthalen-1-yl)-5-mercapto-[1,2,4]triazole; 3-(2,4-Dihydroxy-phenyl)-4-(4-isopropoxy-naphthalen-1-yl)-5-mercapto-[1,2,4]triazole; 3-(2,4-Dihydroxy-phenyl)-4-(5-isopropoxy-naphthalen-1-yl)-5-mercapto-[1,2,4]triazole; 3-(2,4-Dihydroxy-phenyl)-4-(isoquinolin-5-yl)-5-mercapto-[1,2,4]triazole; 3-(2,4-Dihydroxy-phenyl)-4-(5-propoxy-naphthalen-1-yl)-5-mercapto-[1,2,4]triazole; 3-(2-Hydroxy-4-methanesulfonamino-phenyl)-4-(naphthalen-1-yl)-5-mercapto-[1,2,4]triazole; 3-(2,4-Dihydroxy-3,6-dimethyl-phenyl)-4-(naphthalen-1-yl)-5-mercapto-[1,2,4]triazole; 3-(2,4-Dihydroxy-phenyl)-4-[7-(2-methoxyethoxy)-naphthalen-1-yl]-5-mercapto-[1,2,4]triazole; 3-(2,4-Dihydroxy-5-hexyl-phenyl)-4-(naphthalen-1-yl)-5-mercapto-[1,2,4]triazole; 3-(2,4-Dihydroxy-5-ethyl-phenyl)-4-(4-methoxy-naphthalen-1-yl)-5-mercapto-[1,2,4]triazole; 3-(2,4-Dihydroxy-5-ethyl-phenyl)-4-(6-methoxy-naphthalin-1-yl)-5-mercapto-[1,2,4]triazole; 3-(2,4-Dihydroxy-3-chloro-5-ethyl-phenyl)-4-(naphthalen-1-yl)-5-mercapto-[1,2,4]triazole; 3-(2,4-Dihydroxy-5-ethyl-phenyl)-4-(2,3-dimethy-4-methoxy-phenyl)-5-mercapto-[1,2,4]triazole; 3-(2,4-Dihydroxy-phenyl)-4-(7-isopropoxy-naphthalen-1-yl)-5-mercapto-[1,2,4]triazole; 3-(2,4-Dihydroxy-phenyl)-4-(7-ethoxy-naphthalen-1-yl)-5-mercapto-[1,2,4]triazole; 3-(2,4-Dihydroxy-phenyl)-4-(7-propoxy-naphthalen-1-yl)-5-mercapto-[1,2,4]triazole; 3-(2-Hydroxy-4-methoxymethyoxy-phenyl)-4-(naphthalen-1-yl)-5-mercapto-[1,2,4]triazole; 3-[2-Hydroxy-4-(2-hydroxy-ethoxy)-phenyl]-4-(naphthalen-1-yl)-5-mercapto-[1,2,4]triazole; 3-(2,4-Dihydroxyphenyl)-4-(7-methoxy-naphthalen-1-yl)-5-mercapto-[1,2,4]triazole; 3-(2,4-Dihydroxyphenyl)-4-(5-methoxy-naphthalen-1-yl)-5-mercapto-[1,2,4]triazole; 3-(2,4-Dihydroxyphenyl)-4-(4-hydroxy-naphthalen-1-yl)-5-mercapto-[1,2,4]triazole; 3-(2,4-Dihydroxyphenyl)-4-(1-isopropyl-indol-4-yl)-5-mercapto-[1,2,4]triazole; 3-(2,4-Dihydroxy-5-tert-butyl-phenyl)-4-(naphthalen-1-yl)-5-mercapto-[1,2,4]triazole; 3-(2,4-Dihydroxy-5-propyl-phenyl)-4-(naphthalen-1-yl)-5-mercapto-[1,2,4]triazole; 3-(2,4-Dihydroxy-3-methyl-5-ethyl-phenyl)-4-(naphthalen-1-yl)-5-mercapto-[1,2,4]triazole; 3-(2,4-Dihydroxy-5-isobutyl-phenyl)-4-(naphthalen-1-yl)-5-mercapto-[1,2,4]triazole; 3-(2,4-Dihydroxy-phenyl)-4-(2,3-dimethoxy-phenyl)-5-mercapto-[1,2,4]triazole; 3-(2,4-Dihydroxy-phenyl)-4-(2-methoxy-3-chloro-phenyl)-5-mercapto-[1,2,4]triazole; 3-(2,4-Dihydroxy-phenyl)-4-(indol-4-yl)-5-mercapto-[1,2,4]triazole; 3-(2,4-Dihydroxy-phenyl)-4-[1-(2-methoxyethoxy)-indol-4-yl]-5-mercapto-[1,2,4]triazole; 3-(2,4-Dihydroxy-phenyl)-4-(naphthalen-1-yl)-5-hydroxy-[1,2,4]triazole; 3-(1-Oxo-3-hydroxy-pyridin-4-yl)-4-(naphthalen-1-yl)-5-mercapto-[1,2,4]triazole; 3-(2,5-Dihydroxy-4-carboxy)-4-(naphthalen-1-yl)-5-mercapto-[1,2,4]triazole; 3-(2,4-Dihydroxy-5-ethyl-phenyl)-4-(1-isopropyl-indol-4-yl)-5-mercapto-[1,2,4]triazole; 3-(2,4-Dihydroxy-5-ethyl-phenyl)-4-[1-(dimethyl-carbamoyl)-indol-4-yl]-5-mercapto-[1,2,4]triazole; 3-(2,4-Dihydroxy-5-ethyl-phenyl)-4-(1-ethyl-benzoimidazol-4-yl)-5-mercapto-[1,2,4]triazole; 3-(2,4-Dihydroxy-5-ethyl-phenyl)-4-(1,2,3-trimethyl-indol-5-yl)-5-mercapto-[1,2,4]triazole; 3-(2,5-Dihydroxy-4-hydroxymethyl-phenyl)-4-(naphthalen-1-yl)-5-mercapto-[1,2,4]triazole; 3-(2-Hydroxy-4-amino-phenyl)-4-(naphthalen-1-yl)-5-mercapto-[1,2,4]triazole; 3-(2-Hydroxy-4-acetylamino-phenyl)-4-(naphthalen-1-yl)-5-mercapto-[1,2,4]triazole; 3-(2,4-Dihydroxy-3-chloro-phenyl)-4-(naphthalen-1-yl)-5-mercapto-[1,2,4]triazole; 3-(2,4-Dihydroxy-phenyl)-4-(naphthalen-1-yl)-5-mercapto-[1,2,4]triazole; 3-(2,4-Dihydroxy-phenyl)-4-(2-methyl-phenyl)-5-mercapto-[1,2,4]triazole; 3-(2,4-Dihydroxy-phenyl)-4-(2,5-dimethoxy-phenyl)-5-mercapto-[1,2,4]triazole; 3-(2,4-Dihydroxy-phenyl)-4-phenyl-5-mercapto-[1,2,4]triazole; 3-(2-Hydroxy-phenyl)-4-(2-methoxy-phenyl)-5-mercapto-[1,2,4]triazole; 3-(2-Hydroxy-phenyl)-4-(4-methyl-phenyl)-5-mercapto-[1,2,4]triazole; 3-(2-Hydroxy-phenyl)-4-(4-bromo-phenyl)-5-mercapto-[1,2,4]triazole; 3-(2,4-Dihydroxy-phenyl)-4-(naphthalen-1-yl)-5-(methyl sulfanyl)-[1,2,4]triazole; 3-(2,4-Dimethoxy-phenyl)-4-(naphthalen-1-yl)-5-mercapto-[1,2,4]triazole; 3-[2,4-Di-(dimethyl-carbamoyloxy)-phenyl]-4-(naphthalen-1-yl)-5-(dimethyl-carbamoylsulfanyl)-[1,2,4]triazole; 3-(2,4-Dihydroxy-phenyl)-4-(naphthalen-1-yl)-5-(dimethylcarbamoylsulfanyl)-[1,2,4]triazole; 3-(2,4-Diethoxycarbonyloxy-phenyl)-4-(naphthalen-1-yl)-5-(ethoxycarbonylsulfanyl)-[1,2,4]triazole; 3-(2,4-Di-isobutyryloxy-phenyl)-4-(naphthalen-1-yl)-5-(isobutyrylsulfanyl)-[1,2,4]triazole; 3-[2,4-Di-(dimethyl-carbamoyloxy)-phenyl]-4-(quinolin-5-yl)-5-(dimethyl-carbamoylsulfanyl)-[1,2,4]triazole; 3-(2,4-Diacetoxy-phenyl)-4-(naphthalen-1-yl)-5-(acetylsulfanyl)-[1,2,4]triazole; 3-(2,4-Diacetoxy-phenyl)-4-(naphthalen-1-yl)-5-mercapto-[1,2,4]triazole; 3-(2,4-Diethylcarbamoyloxy-phenyl)-4-(naphthalen-1-yl)-5-(ethylcarbamoylsulfanyl)-[1,2,4]triazole; 3-(2,4-Dihydroxy-phenyl)-4-(naphthalen-1-yl)-5-(2-hydroxyethylsulfanyl)-[1,2,4]triazole; 3-(2,4-Dihydroxy-phenyl)-4-ethyl-5-mercapto-[1,2,4]triazole; 3-(2,4-Dihydroxy-phenyl)-4-propyl-5-mercapto-[1,2,4]triazole; 3-(2,4-Dihydroxy-phenyl)-4-isopropyl-5-mercapto-[1,2,4]triazole; 3-(2,4-Dihydroxy-phenyl)-4-butyl-5-mercapto-[1,2,4]triazole; 3-(2,4-Dihydroxy-phenyl)-4-cyclopropyl-5-mercapto-[1,2,4]triazole; 3-(2,4-Dihydroxy-phenyl)-4-(naphthalen-1-yl)-5-(carboxyethysulfanyl)-[1,2,4]triazole; 3-(2,6-Dimethoxy-5-fluoro-pyridin-3-yl)-4-(naphthalen-1-yl)-5-mercapto-[1,2,4]triazole; 3-(2-Methanesulfonyloxy-4-methanesulfonylamino-phenyl)-4-(naphthalen-1-yl)-5-mercapto-[1,2,4]triazole; 3-(2-Methoxy-phenyl)-4-(4-methoxy-phenyl)-5-mercapto-[1,2,4]triazole; 3-(3-Hydroxy-naphthalen-2-yl)-4-phenyl-5-mercapto-[1,2,4]triazole; 3-(2-Methoxy-phenyl)-4-(4-methyl-phenyl)-5-mercapto-[1,2,4]triazole; 3-(2,4-Dihydroxy-5-ethyl-phenyl)-4-(3-methox-phenyl)-5-hydroxy-[1,2,4]triazole; 3-(2,4-Dihydroxy-5-ethyl-phenyl)-4-(naphthalen-1-yl)-5-hydroxy-[1,2,4]triazole; 3-(2,4-Dihydroxy-5-ethyl-phenyl)-4-(1-isopropyl-indol-3-yl)-5-hydroxy-[1,2,4]triazole; 3-(2,4-Dihydroxy-5-ethyl-phenyl)-4-(1-isopropyl-indol-4-yl)-5-amino-[1,2,4]triazole; 3-(2,4-Dihydroxy-5-ethyl-phenyl)-4-(3-methoxy-phenyl)-5-amino-[1,2,4]triazole; 3-(2,4-Dihydroxy-5-ethyl-phenyl)-4-(naphthalen-1-yl)-5-amino-[1,2,4]triazole; 3-(2-Hydroxy-5-ethyloxy-phenyl)-4-(naphthalen-1-yl)-5-hydroxy-[1,2,4]triazole; 3-(2-Hydroxy-5-isopropyl-phenyl)-4-(naphthalen-1-yl)-5-hydroxy-[1,2,4]triazole; 3-(2-Dihydroxy-phenyl)-4-(7-fluoro-naphthalen-1-yl)-5-hydroxy-[1,2,4]triazole; 3-(2,4-Dihydroxy-phenyl)-4-(2,3-difluorophenyl)-5-hydroxy-[1,2,4]triazole; 3-(2,4-Dihydroxy-phenyl)-4-[2-(1H-tetrazol-5-yl)-phenyl]-5-hydroxy-[1,2,4]triazole; 3-(2,4-Dihydroxy-phenyl)-4-(benzothiazol-4-yl)-5-hydroxy-[1,2,4]triazole; 3-(2,4-Dihydroxy-phenyl)-4-(9H-purin-6-yl)-5-hydroxy-[1,2,4]triazole; 3-(2,4-Dihydroxy-phenyl)-4-{4-[2-(moropholin-1-yl)-ethoxy]-phenyl}-5-hydroxy-[1,2,4]triazole; 3-(2,4-Dihydroxy-phenyl)-4-cyclopentyl-5-hydroxy-[1,2,4]triazole; 3-(2,4-Dihydroxy-phenyl)-4-phenyl-5-(sulfamoylamino)-[1,2,4]triazole; 3-(2,4-Dihydroxy-5-methoxy-phenyl)-4-(naphthalene-1-yl)-5-ureido-[1,2,4]triazole; 3-(2,4-Dihydroxy-5-methoxy-phenyl)-4-(2,3-difluorophenyl)-5-ureido-[1,2,4]triazole; 3-(2,4-Dihydroxy-5-ethyl-phenyl)-4-(1-isopropyl-indol-4-yl)-5-ureido-[1,2,4]triazole; 3-(2,4-Dihydroxy-5-ethyl-phenyl)-4-(quinolin-5-yl)-5-ureido-[1,2,4]triazole; 3-(2,4-Dihydroxy-5-methoxy-phenyl)-4-(naphthalene-1-yl)-5-carbamoyloxy-[1,2,4]triazole; 3-(2,4-Dihydroxy-5-ethyl-phenyl)-4-(3-trifluoromethyl-phenyl)-5-carbamoyloxy-[1,2,4]triazole; 3-(2,4-Dihydroxy-5-ethyl-phenyl)-4-(1-methyl-indol-4-yl)-5-carbamoyloxy-[1,2,4]triazole; 3-(2,4-Dihydroxy-5-methoxy-phenyl)-4-(8-methoxy-quinolin-5-yl)-5-carbamoyloxy-[1,2,4]triazole; 3-(2,4-Dihydroxy-5-isopropyl-phenyl)-4-(3-methyl-quinolin-5-yl)-5-carboxyamino-[1,2,4]triazole; 3-(2,4-Dihydroxy-phenyl)-4-(1-methyl-2-chloro-indol-4-yl)-5-carbamoyloxy-[1,2,4]triazole; 3-(2,4-Dihydroxy-5-methoxy-phenyl)-4-[3,5-di-(trifluoromethyl)-phenyl]-5-carbamoyloxy-[1,2,4]triazole; 3-(2,4-Dihydroxy-5-methoxy-phenyl)-4-(3-trifluoromethyl-phenyl)-5-(sulfamoylamino)-[1,2,4]triazole; 3-(2,4-Dihydroxy-5-methoxy-phenyl)-4-(naphthalene-1-yl)-5-(sulfamoylamino)-[1,2,4]triazole; 3-(2,4-Dihydroxy-5-methoxy-phenyl)-4-(1-isopropyl-benzoimidazol-4-yl)-5-(sulfamoylamino)-[1,2,4]triazole; 3-(2,4-Dihydroxy-5-methoxy-phenyl)-4-(3-isopropylphenyl)-5-(thiocarboxyamino)-[1,2,4]triazole; 3-(2,4-Dihydroxy-5-methoxy-phenyl)-4-(3-isopropyloxy-phenyl)-5-(sulfamoyloxy)-[1,2,4]triazole; 3-(2,4-Dihydroxy-5-methoxy-phenyl)-4-(naphthalene-1-yl)-5-(sulfamoyloxy)-[1,2,4]triazole; 3-(2,4-Dihydroxy-5-methoxy-phenyl)-4-(1-isopropyl-benzoimidazol-4-yl)-5-(sulfamoyloxy)-[1,2,4]triazole; 3-(2-Hydroxy-4-ethoxycarbonyoxy-5-methoxy-phenyl)-4-(1-isopropyl-benzoimidazol-4-yl)-5-hydroxy-[1,2,4]triazole; 3-(2-Hydroxy-4-ethoxycarbonyoxy-5-ethyl-phenyl)-4-(naphthalin-2-yl)-5-hydroxy-[1,2,4]triazole; 3-[2-Hydroxy-4-(dimethyl-carbamoyoxy)-5-ethyl-phenyl]-4-(naphthalin-2-yl)-5-hydroxy-[1,2,4]triazole; 3-[2-Hydroxy-4-(dimethyl-carbamoyoxy)-5-chloro-phenyl]-4-(quinolin-5-yl)-5-mercapto-[1,2,4]triazole; 3-[2-Hydroxy-4-(dimethyl-carbamoyoxy)-5-ethyl-phenyl]-4-(2,3-difluoro-phenyl)-5-mercapto-[1,2,4]triazole; 3-[2-Hydroxy-4-isobutyryloxy-5-ethyl-phenyl]-4-(1-methyl-benzo-imidazol-4-yl)-5-hydroxy-[1,2,4]triazole; 3-(2,4-Dihydroxy-5-methoxy-phenyl)-4-(naphthalen-1-yl)-5-mercapto-[1,2,4]triazole; 3-(2,4-Dihydroxy-5-ethyl-phenyl)-4-(5-hydroxy-naphthalen-1-yl)-5-mercapto-[1,2,4]triazole; 3-(2,4-Dihydroxy-phenyl)-4-(naphthalen-1-ylmethyl)-5-mercapto-[1,2,4]triazole; 3-(2-Hydroxy-4-methoxyphenyl)-4-(naphthalen-1-yl)-5-mercapto-[1,2,4]triazole; 3-(2,4-Dihydroxy-phenyl)-4-(biphenyl-3-yl)-5-mercapto-[1,2,4]triazole; 3-(2,4-Dihydroxy-phenyl)-4-(2-methyl-5-hydroxymethyl-phenyl)-5-mercapto-[1,2,4]triazole; 3-(2,4-Dihydroxy-phenyl)-4-(1-dimethylcarbamoyl-indol-4-yl)-5-mercapto-[1,2,4]triazole; 3-(2,4,5-Trihydroxy-phenyl)-4-(naphthalene-1-yl)-5-mercapto-[1,2,4]triazole; 3-(2,4-Dihydroxy-5-ethyl-phenyl)-4-(2,3-dimethyl-indol-5-yl)-5-mercapto-[1,2,4]triazole; 3-(2,4-Dihydroxy-5-ethyl-phenyl)-4-(3-t-butyl-4-methoxy-phenyl)-5-mercapto-[1,2,4]triazole; 3-(2,4-Dihydroxy-5-ethyl-phenyl)-4-(1-ethyl-1H-benzoimidazol-4-yl)-5-mercapto-[1,2,4]triazole, HCl salt; 3-(2,4-Dihydroxy-5-ethyl-phenyl)-4-(1-isopropyl-7-methoxy-indol-4-yl)-5-mercapto-[1,2,4]triazole; 3-(2,4-Dihydroxy-5-cyclopropyl-phenyl)-4-(naphthalene-1-yl)-5-mercapto-[1,2,4]triazole; 3-(2,4-dihydroxy-5-ethyl-phenyl)-4-(1-propyl-indol-4-yl)-5-mercapto-[1,2,4]triazole; 3-(2,4-dihydroxy-5-ethyl-phenyl)-4-(1-acetyl-2,3-dimethyl-indol-5-yl)-5-mercapto-[1,2,4]triazole; 3-(2,4-dihydroxy-5-ethyl-phenyl)-4-(2-methyl-3-ethyl-benzimidazol-5-yl)-5-mercapto-[1,2,4]triazole; 3-(2,4-dihydroxy-5-ethyl-phenyl)-4-(1-ethyl-2-methyl-benzimidazol-5-yl)-5-mercapto-[1,2,4]triazole; 3-(2,4-dihydroxy-5-ethyl-phenyl)-4-(1-propyl-2,3-dimethyl-indol-5-yl)-5-mercapto-[1,2,4]triazole; 3-(2,4-dihydroxy-5-ethyl-phenyl)-4-(N-methyl-tetrahydrocarbozol-7-yl)-5-mercapto-[1,2,4]triazole; 3-(2,4-dihydroxy-5-ethyl-phenyl)-4-(N-methyl-cyclononan[a]indol-5-yl)-5-mercapto-[1,2,4]triazole; 3-(2,4-dihydroxy-5-ethyl-phenyl)-4-(1-n-butyl-indol-4-yl)-5-mercapto-[1,2,4]triazole; 3-(2,4-dihydroxy-5-ethyl-phenyl)-4-(1-n-pentyl-indol-4-yl)-5-mercapto-[1,2,4]triazole; 3-(2,4-dihydroxy-5-ethyl-phenyl)-4-(1-n-hexyl-indol-4-yl)-5-mercapto-[1,2,4]triazole; 3-(2,4-dihydroxy-5-cyclopropyl-phenyl)-4-(1-(1-methylcyclopropyl)-indol-4-yl)-5-mercapto-[1,2,4]triazole; 3-(2,4-dihydroxy-5-cyclopropyl-phenyl)-4-(1-isopropyl-7-methoxy-indol-4-yl)-5-mercapto-[1,2,4]triazole; 3-(2,4-dihydroxy-5-cyclopropyl-phenyl)-4-(1,2,3-trimethyl-indol-5-yl)-5-mercapto-[1,2,4]triazole; 3-(2,4-dihydroxy-5-ethyl-phenyl)-4-(1-isopropyl-7-methoxy-indol-4-yl)-5-mercapto-[1,2,4]triazole disodium salt; 3-(2,4-dihydroxy-5-tert-butyl-phenyl)-4-(1-isopropyl-7-methoxy-indol-4-yl)-5-mercapto-[1,2,4]triazole; 3-(2,4-dihydroxy-5-cyclopropyl-phenyl)-4-(1-propyl-7-methoxy-indol-4-yl)-5-mercapto-[1,2,4]triazole; 3-(2,4-dihydroxy-5-ethyl-phenyl)-4-(1-methyl-3-ethyl-indol-5-yl)-5-mercapto-[1,2,4]triazole; 3-(2,4-dihydroxy-5-ethyl-phenyl)-4-(1,3-dimethyl-indol-5-yl)-5-mercapto-[1,2,4]triazole; 3-(2,4-dihydroxy-5-isopropyl-phenyl)-4-(1-isopropyl-7-methoxy-indol-4-yl)-5-mercapto-[1,2,4]triazole; 3-(2,4-dihydroxy-5-ethyl-phenyl)-4-(1-methyl-3-isopropyl-indol-5-yl)-5-mercapto-[1,2,4]triazole; 3-(2,4-dihydroxy-5-ethyl-phenyl)-4-(N-ethyl-carbozol-7-yl)-5-mercapto-[1,2,4]triazole; 3-(2,4-dihydroxy-5-ethyl-phenyl)-4-(1-isopropyl-7-hydroxy-indol-4-yl)-5-mercapto-[1,2,4]triazole; 3-(2,4-dihydroxy-5-ethyl-phenyl)-4-(1-isopropyl-7-ethoxy-indol-4-yl)-5-mercapto-[1,2,4]triazole; 3-(2,4-dihydroxy-5-ethyl-phenyl)-4-(1,2-dimethyl-indol-5-yl)-5-mercapto-[1,2,4]triazole; 3-(2,4-dihydroxy-5-ethyl-phenyl)-4-(N-methyl-indol-5-yl)-5-mercapto-[1,2,4]triazole; 3-(2,4-dihydroxy-5-ethyl-phenyl)-4-(2-methyl-7-methoxy-benzofuran-4-yl)-5-mercapto-[1,2,4]triazole; 3-(2,4-dihydroxy-5-ethyl-phenyl)-4-(benzofuran-5-yl)-5-mercapto-[1,2,4]triazole; 3-(2,4-dihydroxy-5-ethyl-phenyl)-4-(2-methyl-1,3-benzoxaz-5-yl)-5-mercapto-[1,2,4]triazole; 3-(2,4-dihydroxy-5-isopropyl-phenyl)-4-(1,3-dimethyl-indol-5-yl)-5-mercapto-[1,2,4]triazole; 3-(2,4-dihydroxy-5-cyclopropyl-phenyl)-4-(1,3-dimethyl-indol-5-yl)-5-mercapto-[1,2,4]triazole; 3-(2,4-dihydroxy-5-ethyl-phenyl)-4-(1,3-dimethyl-indol-5-yl)-5-hydroxy-[1,2,4]triazole; 3-(2,4-dihydroxy-5-isopropyl-phenyl)-4-(N-methyl-indol-5-yl)-5-mercapto-[1,2,4]triazole; 3-(2,4-dihydroxy-5-isopropyl-phenyl)-4-(1,2-dimethyl-indol-5-yl)-5-mercapto-[1,2,4]triazole; 3-(2,4-dihydroxy-5-isopropyl-phenyl)-4-(1,3-dimethyl-indol-5-yl)-5-hydroxy-[1,2,4]triazole; 3-(2,4-dihydroxy-5-cyclopropyl-phenyl)-4-(1-methyl-indol-5-yl)-5-mercapto-[1,2,4]triazole; 3-(2,4-dihydroxy-5-isopropyl-phenyl)-4-(1H-indol-5-yl)-5-mercapto-[1,2,4]triazole; 3-(2,4-dihydroxy-5-isopropyl-phenyl)-4-(1-methyl-indol-5-yl)-5-hydroxy-[1,2,4]triazole; 3-(2,4-dihydroxy-5-isopropyl-phenyl)-4-(1-ethyl-indol-5-yl)-5-mercapto-[1,2,4]triazole; 3-(2,4-dihydroxy-5-isopropyl-phenyl)-4-(1-propyl-indol-5-yl)-5-mercapto-[1,2,4]triazole; 3-(2,4-dihydroxy-5-isopropyl-phenyl)-4-(1-methyl-2-trifluoromethyl-benzimidazol-5-yl)-5-mercapto-[1,2,4]triazole; 3-(2,4-dihydroxy-5-isopropyl-phenyl)-4-(1-methyl-indazol-5-yl)-5-mercapto-[1,2,4]triazole; 3-(2,4-dihydroxy-5-isopropyl-phenyl)-4-(1-methyl-indazol-6-yl)-5-mercapto-[1,2,4]triazole; 3-(2,4-dihydroxy-5-isopropyl-phenyl)-4-(1-isopropyl-indol-4-yl)-5-hydroxy-[1,2,4]triazole; 3-(2,4-dihydroxy-5-isopropyl-phenyl)-4-(1,3-benzodiaxo1-5-yl)-5-mercapto-[1,2,4]triazole; 3-(2,4-dihydroxy-5-isopropyl-phenyl)-4-(indan-5-yl)-5-mercapto-[1,2,4]triazole; 3-(2,4-dihydroxy-5-isopropyl-phenyl)-4-(2-methyl-indazol-6-yl)-5-mercapto-[1,2,4]triazole; 3-(2,4-dihydroxy-5-ethyl-phenyl)-4-(3-oxo-benzo[1,4]oxazin-6-yl)-5-mercapto-[1,2,4]triazole; 3-(2,4-dihydroxy-5-ethyl-phenyl)-4-(2-oxo-1,3-dihydro-benzoimidazol-5-yl)-5-mercapto-[1,2,4]triazole; 3-(2,4-dihydroxy-5-isopropyl-phenyl)-4-(2H-benzo[1,4]oxazin-6-yl)-5-mercapto-[1,2,4]triazole; 4-Ethyl-6-[5-mercapto-4-(1-methyl-2,3-dihydro-1H-indol-5-yl)-4H-[1,2,4]triazol-3-yl]-benzene-1,3-diol; 5-(3-(5-ethyl-2,4-dihydroxyphenyl)-5-mercapto-4H-1,2,4-triazol-4-yl)indolin-2-one; 5-(3-(5-ethyl-2,4-dihydroxyphenyl)-5-mercapto-4H-1,2,4-triazol-4-yl)-1H-benzo[d]imidazol-2(3H)-one; 5-(3-(5-ethyl-2,4-dihydroxyphenyl)-5-mercapto-4H-1,2,4-triazol-4-yl)-1-methylindolin-2-one; 4-isopropyl-6-(5-mercapto-4-(4-propyl-3,4-dihydro-2H-benzo[b][1,4]oxazin-6-yl)-4H-1,2,4-triazol-3-yl)benzene-1,3-diol; 6-(3-(5-ethyl-2,4-dihydroxyphenyl)-5-mercapto-4H-1,2,4-triazol-4-yl)-2H-benzo[b][1,4]oxazin-3(4H)-one; 6-(3-(5-ethyl-2,4-dihydroxyphenyl)-5-mercapto-4H-1,2,4-triazol-4-yl)-3-methylbenzo-[d]thiazol-2(3H)-one; and 6-(3-(5-ethyl-2,4-dihydroxyphenyl)-5-mercapto-4H-1,2,4-triazol-4-yl)benzo[d]thiazol-2(3H)-one; or a tautomer or a pharmaceutically acceptable salt thereof.

In another embodiment of the method, the Hsp90 inhibitor is selected from 3-(2,4-Dihydroxy-5-ethyl-phenyl)-4-(naphthalen-1-yl)-5-mercapto-[1,2,4]triazole; 3-(2,4-Dihydroxy-5-ethyl-phenyl)-4-(1-isopropyl-7-methoxy-indol-4-yl)-5-mercapto-[1,2,4]triazole; 3-(2,4-dihydroxy-5-isopropyl-phenyl)-4-(N-methyl-indol-5-yl)-5-mercapto-[1,2,4]triazole; 3-(2,4-dihydroxy-5-isopropyl-phenyl)-4-(1-methyl-indol-5-yl)-5-hydroxy-[1,2,4]triazole; or a tautomer or pharmaceutically acceptable salt thereof.

In another embodiment of the method, the Hsp90 inhibitor is 3-(2,4-dihydroxy-5-isopropyl-phenyl)-4-(1-methyl-indol-5-yl)-5-hydroxy-[1,2,4]triazole; or a tautomer or pharmaceutically acceptable salt thereof.

### i) Exemplary Compounds Disclosed Herein

Exemplary compounds described herein are depicted in Table 1 below, including tautomers and pharmaceutically acceptable salts thereof.

TABLE 1

| | | Tautomeric | |
|---|---|---|---|
| No. | Structure | Structure | Name |
| 1 | 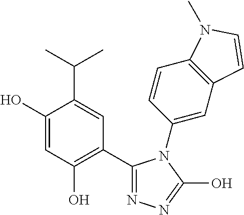 [SMILES: CC(C)c1cc(-c2nnc(O)n2-c2ccc3c(ccn3C)c2)c(O)cc1O] | 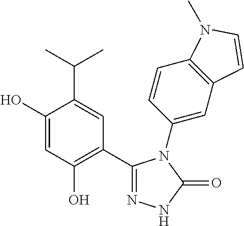 [SMILES: CC(C)c1cc(-c2n[nH]c(=O)n2-c2ccc3c(ccn3C)c2)c(O)cc1O] | 3-(2,4-dihydroxy-5- isopropyl-phenyl)-4-(1- methyl-indol-5-yl)-5- hydroxy-[1,2,4] triazole |
| 2 | 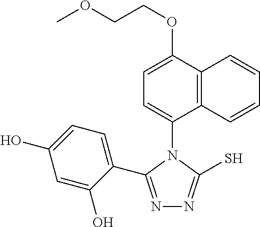 [SMILES: COCCOc1ccc(-n2c(S)nnc2-c2ccc(O)cc2O)c2ccccc12] | 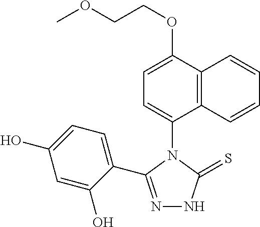 [SMILES: COCCOc1ccc(-n2c(-c3ccc(O)cc3O)n[nH]c2=S)c2ccccc12] | 3-(2,4-Dihydroxyphenyl)-4- [4-(2-methoxyethoxy)- naphthalen-1-yl]-5-mercapto- [1,2,4] triazole |
| 3 | 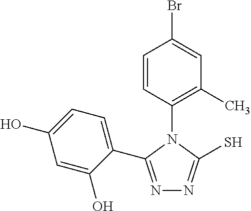 [SMILES: Cc1cc(Br)ccc1-n1c(S)nnc1-c1ccc(O)cc1O] | 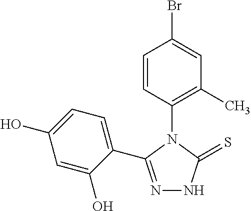 [SMILES: Cc1cc(Br)ccc1-n1c(-c2ccc(O)cc2O)n[nH]c1=S] | 3-(2,4-Dihydroxyphenyl)-4- (2-methyl-4-bromophenyl)-5- mercapto-[1,2,4] triazole |
| 4 | 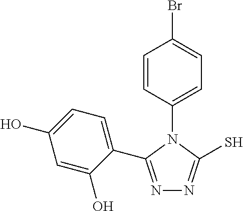 [SMILES: Oc1ccc(-c2nnc(S)n2-c2ccc(Br)cc2)c(O)c1] | 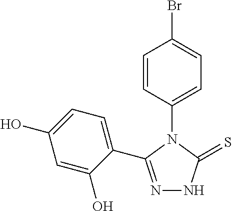 [SMILES: Oc1ccc(-c2n[nH]c(=S)n2-c2ccc(Br)cc2)c(O)c1] | 3-(2,4-Dihydroxyphenyl)-4- (4-bromophenyl)-5- mercapto-[1,2,4] triazole |
| 5 | 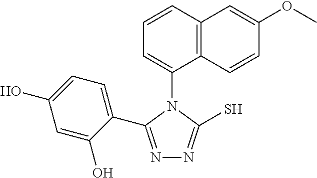 [SMILES: COc1ccc2c(-n3c(S)nnc3-c3ccc(O)cc3O)cccc2c1] | 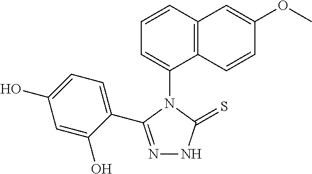 [SMILES: COc1ccc2c(-n3c(-c4ccc(O)cc4O)n[nH]c3=S)cccc2c1] | 3-(3,4-Dihydroxyphenyl)-4- (6-methoxy-naphthalen-1-yl)- 5-mercapto-[1,2,4] triazole |
| 6 | 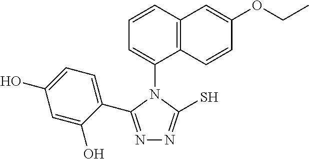 [SMILES: CCOc1ccc2c(-n3c(S)nnc3-c3ccc(O)cc3O)cccc2c1] | 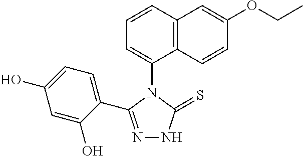 [SMILES: CCOc1ccc2c(-n3c(-c4ccc(O)cc4O)n[nH]c3=S)cccc2c1] | 3-(3,4-Dihydroxyphenyl)-4- (6-ethoxy-naphthalen-1-yl)- 5-mercapto-[1,2,4] triazole |
| 7 | 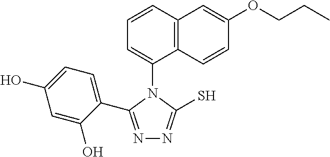 [SMILES: CCCOc1ccc2c(-n3c(S)nnc3-c3ccc(O)cc3O)cccc2c1] | 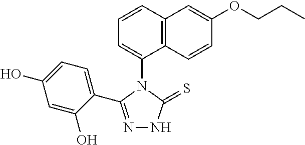 [SMILES: CCCOc1ccc2c(-n3c(-c4ccc(O)cc4O)n[nH]c3=S)cccc2c1] | 3-(3,4-Dihydroxyphenyl)-4- (6-propoxy-naphthalen-1-yl)- 5-mercapto-[1,2,4] triazole |
| 8 | 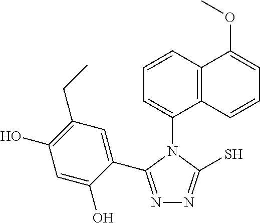 [SMILES: CCc1cc(-c2nnc(S)n2-c2cccc3c(OC)cccc23)c(O)cc1O] | 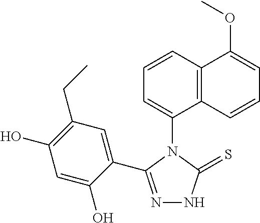 [SMILES: CCc1cc(-c2n[nH]c(=S)n2-c2cccc3c(OC)cccc23)c(O)cc1O] | 3-(2,4-Dihydroxy-5-ethyl- phenyl)-4-(5-methoxy- naphthalen-1-yl)-5-mercapto- [1,2,4] triazole |
| 9 | 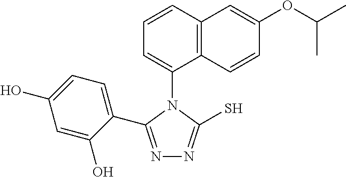 [SMILES: CC(C)Oc1ccc2c(-n3c(S)nnc3-c3ccc(O)cc3O)cccc2c1] | 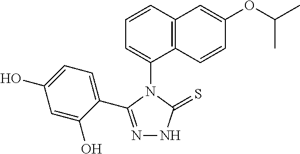 [SMILES: CC(C)Oc1ccc2c(-n3c(-c4ccc(O)cc4O)n[nH]c3=S)cccc2c1] | 3-(3,4-Dihydroxyphenyl)-4- (6-isopropoxy-naphthalen-1- yl)-5-mercapto-[1,2,4] triazole |
| 10 | 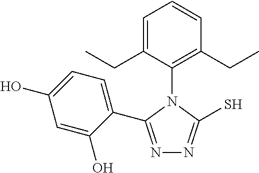 [SMILES: CCc1cccc(CC)c1-n1c(S)nnc1-c1ccc(O)cc1O] | 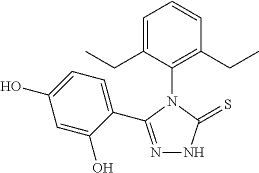 [SMILES: CCc1cccc(CC)c1-n1c(-c2ccc(O)cc2O)n[nH]c1=S] | 3-(2,4-Dihydroxyphenyl)-4- (2,6-diethylphenyl)-5- mercapto-[1,2,4] triazole |
| 11 | 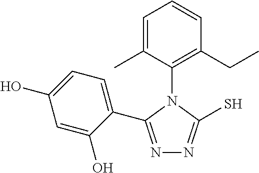 [SMILES: CCc1cccc(C)c1-n1c(S)nnc1-c1ccc(O)cc1O] | 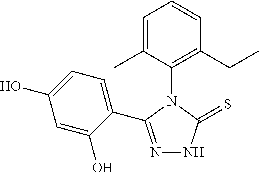 [SMILES: CCc1cccc(C)c1-n1c(-c2ccc(O)cc2O)n[nH]c1=S] | 3-(2,4-Dihydroxyphenyl)-4- (2-methy-6-ethylphenyl)-5- mercapto-[1,2,4] triazole |
| 12 | 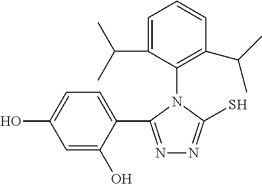 [SMILES: CC(C)c1cccc(C(C)C)c1-n1c(S)nnc1-c1ccc(O)cc1O] | 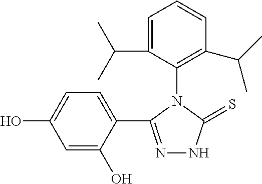 [SMILES: CC(C)c1cccc(C(C)C)c1-n1c(-c2ccc(O)cc2O)n[nH]c1=S] | 3-(2,4-Dihydroxyphenyl)-4- (2,6-diisopropylphenyl)-5- mercapto-[1,2,4] triazole |
| 13 | 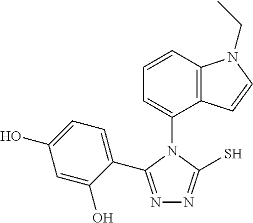 [SMILES: CCn1ccc2c(-n3c(S)nnc3-c3ccc(O)cc3O)cccc21] | 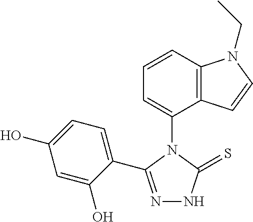 [SMILES: CCn1ccc2c(-n3c(-c4ccc(O)cc4O)n[nH]c3=S)cccc21] | 3-(2,4-Dihydroxyphenyl)-4- (1-ethyl-indol-4-yl)-5- mercapto-[1,2,4] triazole |
| 14 | 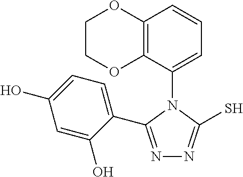 [SMILES: Oc1ccc(-c2nnc(S)n2-c2cccc3c2OCCO3)c(O)c1] | 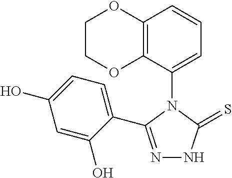 [SMILES: Oc1ccc(-c2n[nH]c(=S)n2-c2cccc3c2OCCO3)c(O)c1] | 3-(2,4-Dihydroxyphenyl)-4- (2,3-dihydro- benzo[1,4]dioxin-5-yl)-5- mercapto-[1,2,4] triazole |
| 15 | 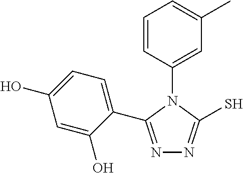 [SMILES: Cc1cccc(-n2c(S)nnc2-c2ccc(O)cc2O)c1] | 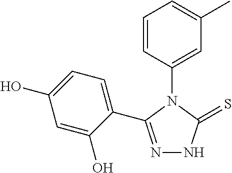 [SMILES: Cc1cccc(-n2c(-c3ccc(O)cc3O)n[nH]c2=S)c1] | 3-(2,4-Dihydroxyphenyl)-4- (3-methylphenyl)-5- mercapto-[1,2,4] triazole |
| 16 | 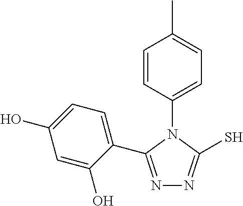 [SMILES: Cc1ccc(-n2c(S)nnc2-c2ccc(O)cc2O)cc1] | 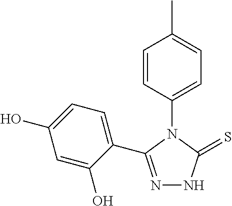 [SMILES: Cc1ccc(-n2c(-c3ccc(O)cc3O)n[nH]c2=S)cc1] | 3-(2,4-Dihydroxyphenyl)-4- (4-methylphenyl)-5- mercapto-[1,2,4] triazole |
| 17 | 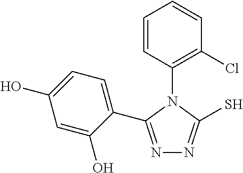 [SMILES: Oc1ccc(-c2nnc(S)n2-c2ccccc2Cl)c(O)c1] | 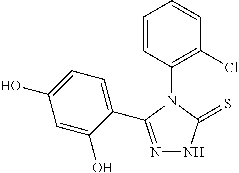 [SMILES: Oc1ccc(-c2n[nH]c(=S)n2-c2ccccc2Cl)c(O)c1] | 3-(2,4-Dihydroxyphenyl)-4- (2-chlorophenyl)-5-mercapto- [1,2,4] triazole |
| 18 | 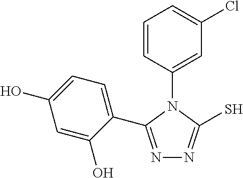 [SMILES: Oc1ccc(-c2nnc(S)n2-c2cccc(Cl)c2)c(O)c1] | 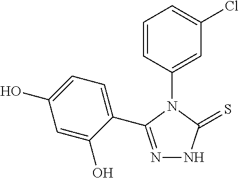 [SMILES: Oc1ccc(-c2n[nH]c(=S)n2-c2cccc(Cl)c2)c(O)c1] | 3-(2,4-Dihydroxyphenyl)-4- (3-chlorophenyl)-5-mercapto- [1,2,4] triazole |
| 19 | 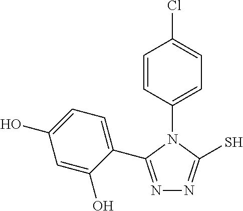 [SMILES: Oc1ccc(-c2nnc(S)n2-c2ccc(Cl)cc2)c(O)c1] | 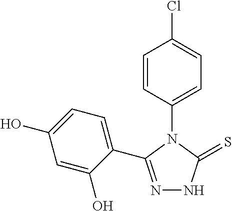 [SMILES: Oc1ccc(-c2n[nH]c(=S)n2-c2ccc(Cl)cc2)c(O)c1] | 3-(2,4-Dihydroxyphenyl)-4- (4-chlorophenyl)-5-mercapto- [1,2,4] triazole |
| 20 | 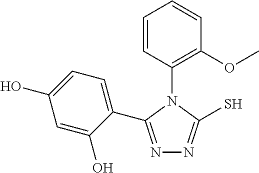 [SMILES: COc1ccccc1-n1c(S)nnc1-c1ccc(O)cc1O] | 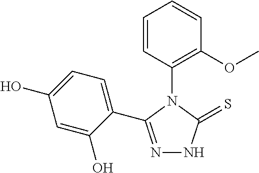 [SMILES: COc1ccccc1-n1c(-c2ccc(O)cc2O)n[nH]c1=S] | 3-(2,4-Dihydroxyphenyl)-4- (2-methoxyphenyl)-5- mercapto-[1,2,4] triazole |
| 21 | 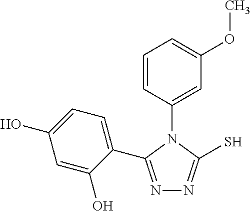 [SMILES: COc1cccc(-n2c(S)nnc2-c2ccc(O)cc2O)c1] | 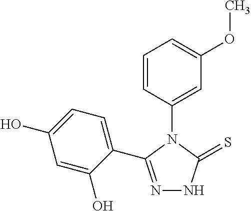 [SMILES: COc1cccc(-n2c(-c3ccc(O)cc3O)n[nH]c2=S)c1] | 3-(2,4-Dihydroxyphenyl)-4- (3-methoxyphenyl)-5- mercapto-[1,2,4] triazole |
| 22 | 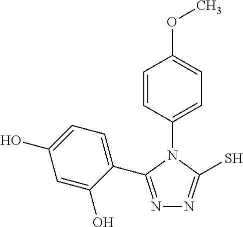 [SMILES: COc1ccc(-n2c(S)nnc2-c2ccc(O)cc2O)cc1] | 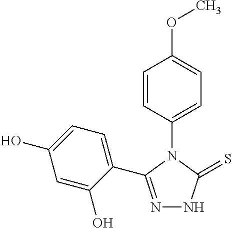 [SMILES: COc1ccc(-n2c(-c3ccc(O)cc3O)n[nH]c2=S)cc1] | 3-(2,4-Dihydroxyphenyl)-4- (4-methoxyphenyl)-5- mercapto-[1,2,4] triazole |
| 23 | 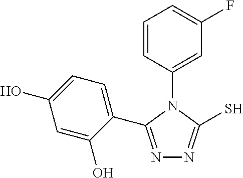 [SMILES: Oc1ccc(-c2nnc(S)n2-c2cccc(F)c2)c(O)c1] | 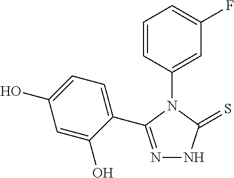 [SMILES: Oc1ccc(-c2n[nH]c(=S)n2-c2cccc(F)c2)c(O)c1] | 3-(2,4-Dihydroxyphenyl)-4- (3-fluorophenyl)-5-mercapto- [1,2,4] triazole |
| 24 | 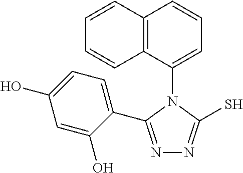 [SMILES: Oc1ccc(-c2nnc(S)n2-c2cccc3ccccc23)c(O)c1] | 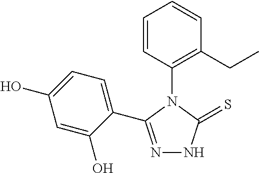 [SMILES: CCc1ccccc1-n1c(-c2ccc(O)cc2O)n[nH]c1=S] | 3-(2,4-Dihydroxyphenyl)-4- (2-ethylphenyl)-5-mercapto- [1,2,4] triazole |
| 25 | 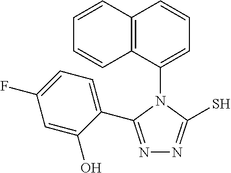 [SMILES: Oc1cc(F)ccc1-c1nnc(S)n1-c1cccc2ccccc12] | 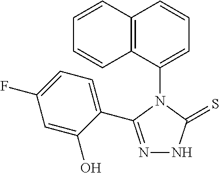 [SMILES: Oc1cc(F)ccc1-c1n[nH]c(=S)n1-c1cccc2ccccc12] | 3-(2-Hydroxy-4- fluorophenyl)-4-(naphthalen- 1-yl)-5-mercapto-[1,2,4] triazole |
| 26 | 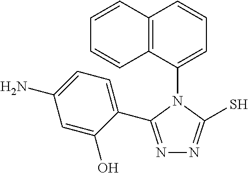 [SMILES: Nc1ccc(-c2nnc(S)n2-c2cccc3ccccc23)c(O)c1] | 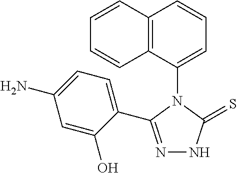 [SMILES: Nc1ccc(-c2n[nH]c(=S)n2-c2cccc3ccccc23)c(O)c1] | 3-(2-Hydroxy-4- aminophenyl)-4-(naphthalen- 1-yl)-5-mercapto-[1,2,4] triazole |
| 27 | 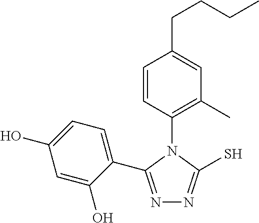 [SMILES: CCCCc1ccc(-n2c(S)nnc2-c2ccc(O)cc2O)c(C)c1] | 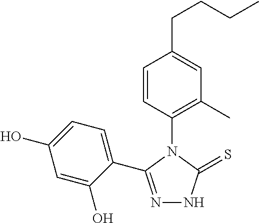 [SMILES: CCCCc1ccc(-n2c(-c3ccc(O)cc3O)n[nH]c2=S)c(C)c1] | 3-(2,4-Dihydroxyphenyl)-4- (2-methyl-4-butyl-phenyl)-5- mercapto-[1,2,4] triazole |
| 28 | 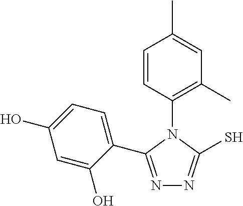 [SMILES: Cc1ccc(-n2c(S)nnc2-c2ccc(O)cc2O)c(C)c1] | 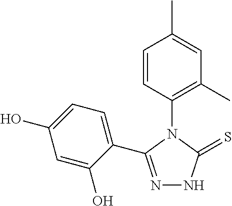 [SMILES: Cc1ccc(-n2c(-c3ccc(O)cc3O)n[nH]c2=S)c(C)c1] | 3-(2,4-Dihydroxyphenyl)-4- (2,4-dimethyl-phenyl)-5- mercapto-[1,2,4] triazole |
| 29 | 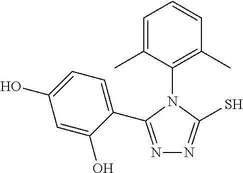 [SMILES: Cc1cccc(C)c1-n1c(S)nnc1-c1ccc(O)cc1O] | 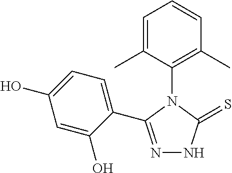 [SMILES: Cc1cccc(C)c1-n1c(-c2ccc(O)cc2O)n[nH]c1=S] | 3-(2,4-Dihydroxyphenyl)-4- (2,6-dimethyl-phenyl)-5- mercapto-[1,2,4] triazole |
| 30 | 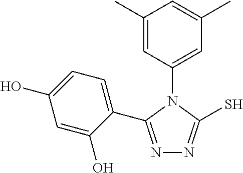 [SMILES: Cc1cc(C)cc(-n2c(S)nnc2-c2ccc(O)cc2O)c1] | 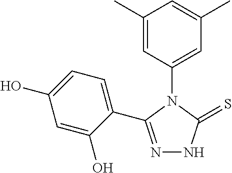 [SMILES: Cc1cc(C)cc(-n2c(-c3ccc(O)cc3O)n[nH]c2=S)c1] | 3-(2,4-Dihydroxyphenyl)-4- (2,6-dimethyl-phenyl)-5- mercapto-[1,2,4] triazole |
| 31 | 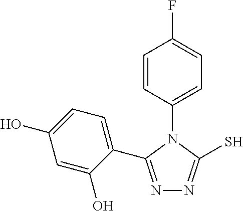 [SMILES: Oc1ccc(-c2nnc(S)n2-c2ccc(F)cc2)c(O)c1] | 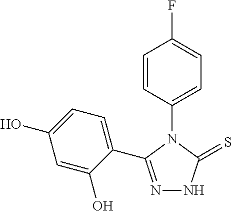 [SMILES: Oc1ccc(-c2n[nH]c(=S)n2-c2ccc(F)cc2)c(O)c1] | 3-(2,4-Dihydroxyphenyl)-4- (4-fluorophenyl)-5-mercapto- [1,2,4] triazole |
| 32 | 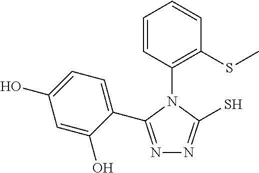 [SMILES: CSc1ccccc1-n1c(S)nnc1-c1ccc(O)cc1O] | 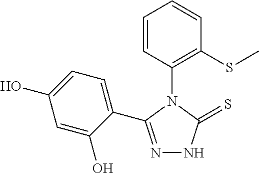 [SMILES: CSc1ccccc1-n1c(-c2ccc(O)cc2O)n[nH]c1=S] | 3-(2,4-Dihydroxyphenyl)-4- (2-methylsulfanylphenyl)-5- mercapto-[1,2,4] triazole |
| 33 | 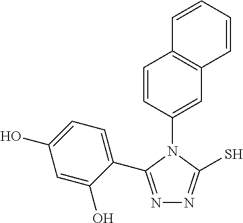 [SMILES: Oc1ccc(-c2nnc(S)n2-c2ccc3ccccc3c2)c(O)c1] | 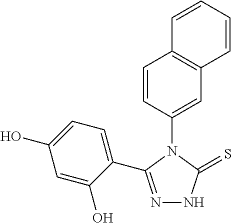 [SMILES: Oc1ccc(-c2n[nH]c(=S)n2-c2ccc3ccccc3c2)c(O)c1] | 3-(2,4-Dihydroxyphenyl)-4- (naphthalene-2-yl)-5- mercapto-[1,2,4] triazole |
| 34 | 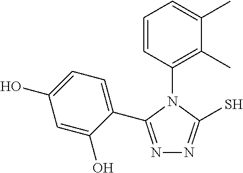 [SMILES: Cc1cccc(-n2c(S)nnc2-c2ccc(O)cc2O)c1C] | 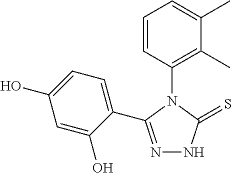 [SMILES: Cc1cccc(-n2c(-c3ccc(O)cc3O)n[nH]c2=S)c1C] | 3-(2,4-Dihydroxyphenyl)-4- (2,3-dimethylphenyl)-5- mercapto-[1,2,4] triazole |
| 35 | 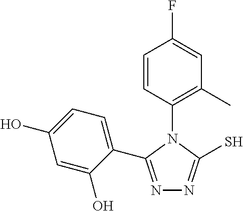 [SMILES: Cc1cc(F)ccc1-n1c(S)nnc1-c1ccc(O)cc1O] | 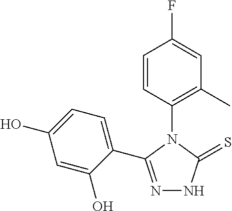 [SMILES: Cc1cc(F)ccc1-n1c(-c2ccc(O)cc2O)n[nH]c1=S] | 3-(2,4-Dihydroxyphenyl)-4- (2-methyl-4-fluorophenyl)-5- mercapto-[1,2,4] triazole |
| 36 | 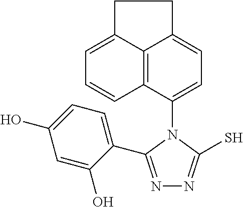 [SMILES: Oc1ccc(-c2nnc(S)n2-c2ccc3c4c(cccc24)CC3)c(O)c1] | 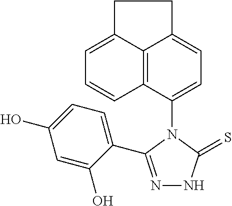 [SMILES: Oc1ccc(-c2n[nH]c(=S)n2-c2ccc3c4c(cccc24)CC3)c(O)c1] | 3-(2,4-Dihydroxyphenyl)-4- (acenaphthalen-5-yl)-5- mercapto-[1,2,4] triazole |
| 37 | 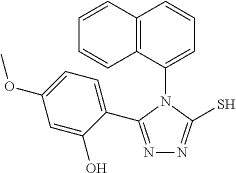 [SMILES: COc1ccc(-c2nnc(S)n2-c2cccc3ccccc23)c(O)c1] | 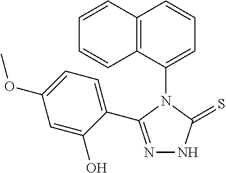 [SMILES: COc1ccc(-c2n[nH]c(=S)n2-c2cccc3ccccc23)c(O)c1] | 3-(2-Hydroxy-4-methoxy- phenyl)-4-(naphthalen-1-yl)- 5-mercapto-[1,2,4] triazole |
| 38 | 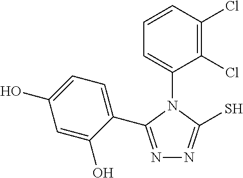 [SMILES: Oc1ccc(-c2nnc(S)n2-c2cccc(Cl)c2Cl)c(O)c1] | 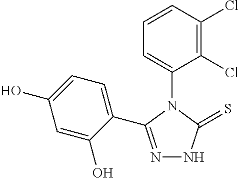 [SMILES: Oc1ccc(-c2n[nH]c(=S)n2-c2cccc(Cl)c2Cl)c(O)c1] | 3-(2,4-Dihydroxyphenyl)-4- (2,3-dichlorophenyl)-5- mercapto-[1,2,4] triazole |
| 39 | 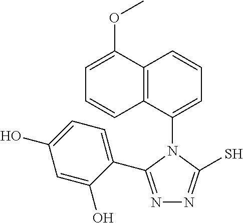 [SMILES: COc1cccc2c(-n3c(S)nnc3-c3ccc(O)cc3O)cccc12] | 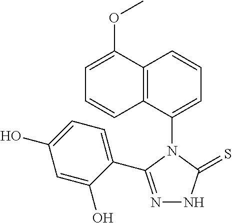 [SMILES: COc1cccc2c(-n3c(-c4ccc(O)cc4O)n[nH]c3=S)cccc12] | 3-(2,4-Dihydroxyphenyl)-4- (5-methoxynaphthalen-1-yl)- 5-mercapto-[1,2,4] triazole |
| 40 | 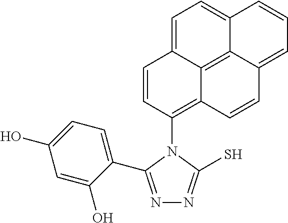 [SMILES: Oc1ccc(-c2nnc(S)n2-c2ccc3ccc4cccc5ccc2c3c45)c(O)c1] | 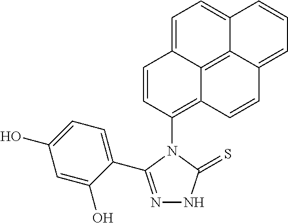 [SMILES: Oc1ccc(-c2n[nH]c(=S)n2-c2ccc3ccc4cccc5ccc2c3c45)c(O)c1] | 3-(2,4-Dihydroxyphenyl)-4- (pyren-1-yl)-5-mercapto- [1,2,4] triazole |
| 41 | 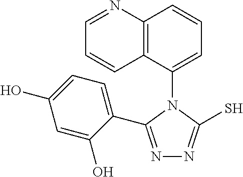 [SMILES: Oc1ccc(-c2nnc(S)n2-c2cccc3ncccc23)c(O)c1] | 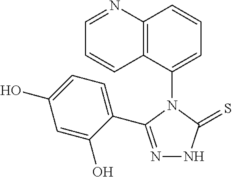 [SMILES: Oc1ccc(-c2n[nH]c(=S)n2-c2cccc3ncccc23)c(O)c1] | 3-(2,4-Dihydroxyphenyl)-4- (quinolin-5-yl)-5-mercapto- [1,2,4] triazole |
| 42 | 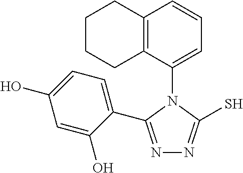 [SMILES: Oc1ccc(-c2nnc(S)n2-c2cccc3c2CCCC3)c(O)c1] | 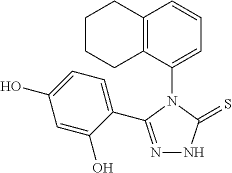 [SMILES: Oc1ccc(-c2n[nH]c(=S)n2-c2cccc3c2CCCC3)c(O)c1] | 3-(2,4-Dihydroxyphenyl)-4- (1,2,3,4- tetrahydronaphthalen-5-yl)-5- mercapto-[1,2,4] triazole |
| 43 | 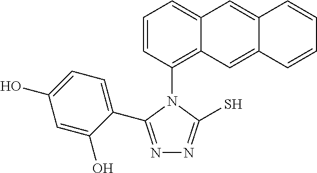 [SMILES: Oc1ccc(-c2nnc(S)n2-c2cccc3cc4ccccc4cc23)c(O)c1] | 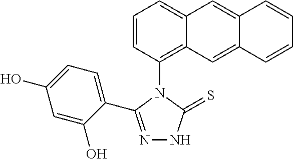 [SMILES: Oc1ccc(-c2n[nH]c(=S)n2-c2cccc3cc4ccccc4cc23)c(O)c1] | 3-(2,4-Dihydroxyphenyl)-4- (anthracen-1-yl)-5-mercapto- [1,2,4] triazole |
| 44 | 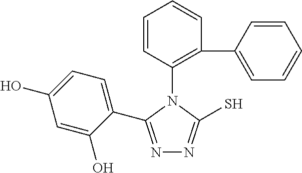 [SMILES: Oc1ccc(-c2nnc(S)n2-c2ccccc2-c2ccccc2)c(O)c1] | 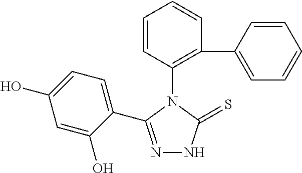 [SMILES: Oc1ccc(-c2n[nH]c(=S)n2-c2ccccc2-c2ccccc2)c(O)c1] | 3-(2,4-Dihydroxyphenyl)-4- (biphenyl-2-yl)-5-mercapto- [1,2,4] triazole |
| 45 | 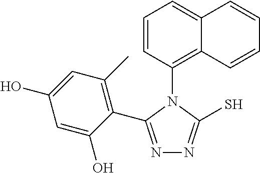 [SMILES: Cc1cc(O)cc(O)c1-c1nnc(S)n1-c1cccc2ccccc12] | 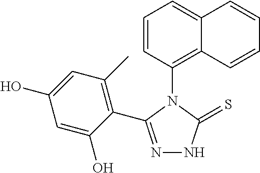 [SMILES: Cc1cc(O)cc(O)c1-c1n[nH]c(=S)n1-c1cccc2ccccc12] | 3-(2,4-Dihydroxy-6-methyl- phenyl)-4-(naphthalene-1-yl)- 5-mercapto-[1,2,4] triazole |
| 46 | 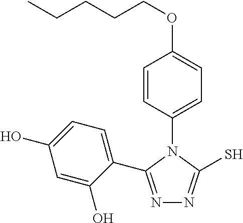 [SMILES: CCCCCOc1ccc(-n2c(S)nnc2-c2ccc(O)cc2O)cc1] | 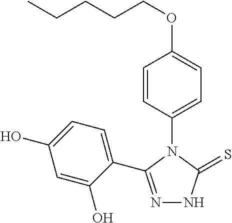 [SMILES: CCCCCOc1ccc(-n2c(-c3ccc(O)cc3O)n[nH]c2=S)cc1] | 3-(2,4-Dihydroxyphenyl)-4- (4-pentyloxyphenyl)-5- mercapto-[1,2,4] triazole |
| 47 | 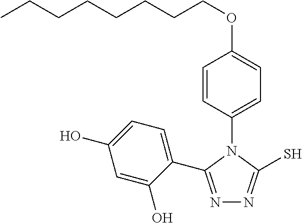 [SMILES: CCCCCCCCOc1ccc(-n2c(S)nnc2-c2ccc(O)cc2O)cc1] | 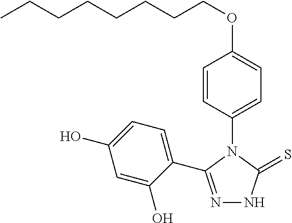 [SMILES: CCCCCCCCOc1ccc(-n2c(-c3ccc(O)cc3O)n[nH]c2=S)cc1] | 3-(2,4-Dihydroxyphenyl)-4- (4-octyloxyphenyl)-5- mercapto-[1,2,4] triazole |
| 48 | 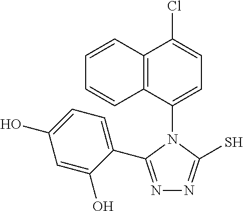 [SMILES: Oc1ccc(-c2nnc(S)n2-c2ccc(Cl)c3ccccc23)c(O)c1] | 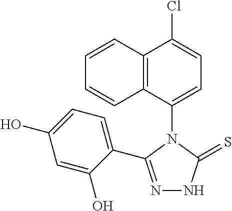 [SMILES: Oc1ccc(-c2n[nH]c(=S)n2-c2ccc(Cl)c3ccccc23)c(O)c1] | 3-(2,4-Dihydroxyphenyl)-4- (4-chloronaphthalen-1-yl)-5- mercapto-[1,2,4] triazole |
| 49 | 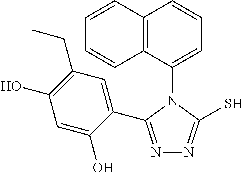 [SMILES: CCc1cc(-c2nnc(S)n2-c2cccc3ccccc23)c(O)cc1O] | 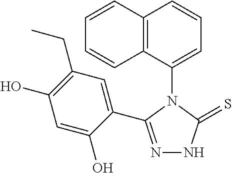 [SMILES: CCc1cc(-c2n[nH]c(=S)n2-c2cccc3ccccc23)c(O)cc1O] | 3-(2,4-Dihydroxy-5-ethyl- phenyl)-4-(naphthalen-1-yl)- 5-mercapto-[1,2,4] triazole |
| 50 | 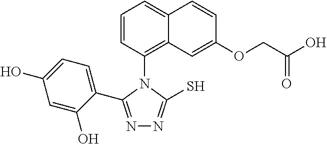 [SMILES: O=C(O)COc1ccc2cccc(-n3c(S)nnc3-c3ccc(O)cc3O)c2c1] | 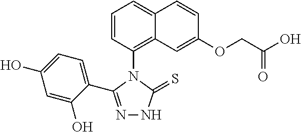 [SMILES: O=C(O)COc1ccc2cccc(-n3c(-c4ccc(O)cc4O)n[nH]c3=S)c2c1] | 3-(2,4-Dihydroxy-5-ethyl- phenyl)-4-(7- carboxymethoxy-naphthalen- 1-yl)-5-mercapto-[1,2,4] triazole |
| 51 | 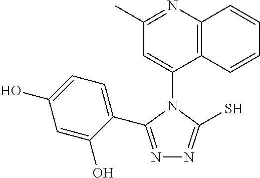 [SMILES: Cc1cc(-n2c(S)nnc2-c2ccc(O)cc2O)c2ccccc2n1] | 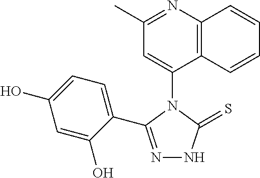 [SMILES: Cc1cc(-n2c(-c3ccc(O)cc3O)n[nH]c2=S)c2ccccc2n1] | 3-(2,4-Dihydroxyphenyl)-4- (2-methyl-quinolin-4-yl)-5- mercapto-[1,2,4] triazole |
| 52 | 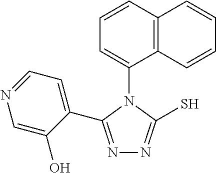 [SMILES: Oc1cnccc1-c1nnc(S)n1-c1cccc2ccccc12] | 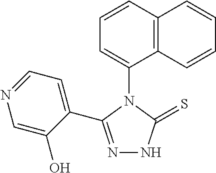 [SMILES: Oc1cnccc1-c1n[nH]c(=S)n1-c1cccc2ccccc12] | 3-(3-Hydroxypyridin-4-yl)-4- (naphthalen-1-yl)-5- mercapto-[1,2,4] triazole |
| 53 | 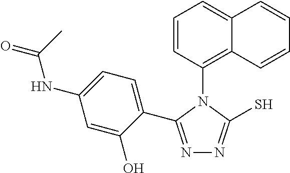 [SMILES: CC(=O)Nc1ccc(-c2nnc(S)n2-c2cccc3ccccc23)c(O)c1] | 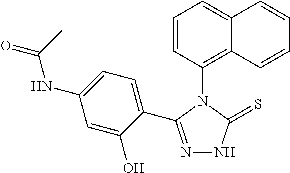 [SMILES: CC(=O)Nc1ccc(-c2n[nH]c(=S)n2-c2cccc3ccccc23)c(O)c1] | 3-(2-Hydroxy-4-acetylamino- phenyl)-4-(naphthalen-1-yl)- 5-mercapto-[1,2,4] triazole |
| 54 | 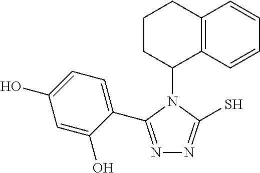 [SMILES: Oc1ccc(-c2nnc(S)n2C2CCCc3ccccc32)c(O)c1] | 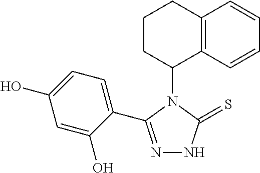 [SMILES: Oc1ccc(-c2n[nH]c(=S)n2C2CCCc3ccccc32)c(O)c1] | 3-(2,4-Dihydroxy-phenyl)-4- (1,2,3,4- tetrahydronaphlhalen-1-yl)-5- mercapto-[1,2,4] triazole |
| 55 | 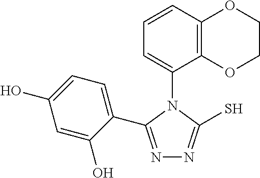 [SMILES: Oc1ccc(-c2nnc(S)n2-c2cccc3c2OCCO3)c(O)c1] | 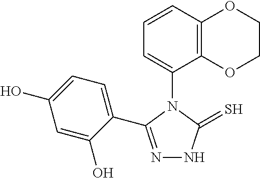 [SMILES: Oc1ccc(-c2n[nH]c(=S)n2-c2cccc3c2OCCO3)c(O)c1] | 3-(2,4-Dihydroxy-phenyl)-4- (2,3-dihydro- benzo[1,4]dioxin-5-yl)-5- mercapto-[1,2,4] triazole |
| 56 | 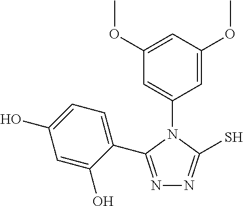 [SMILES: COc1cc(OC)cc(-n2c(S)nnc2-c2ccc(O)cc2O)c1] | 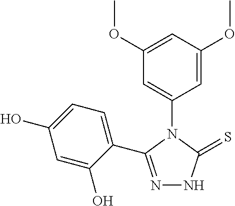 [SMILES: COc1cc(OC)cc(-n2c(-c3ccc(O)cc3O)n[nH]c2=S)c1] | 3-(2,4-Dihydroxy-phenyl)-4- (3,5-dimethoxyphenyl)-5- mercapto-[1,2,4] triazole |
| 57 | 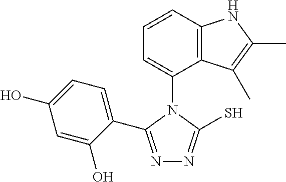 [SMILES: Cc1[nH]c2cccc(-n3c(S)nnc3-c3ccc(O)cc3O)c2c1C] | 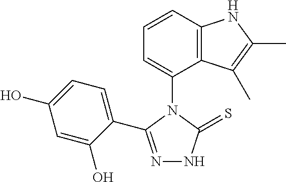 [SMILES: Cc1[nH]c2cccc(-n3c(-c4ccc(O)cc4O)n[nH]c3=S)c2c1C] | 3-(2,4-Dihydroxy-phenyl)-4- (2,3-dimethyl-1H-indol-4-yl)- 5-mercapto-[1,2,4] triazole |
| 58 | 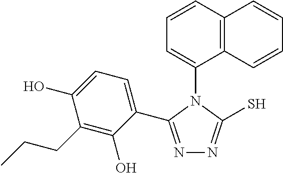 [SMILES: CCCc1c(O)ccc(-c2nnc(S)n2-c2cccc3ccccc23)c1O] | 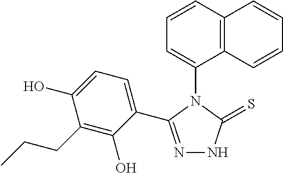 [SMILES: CCCc1c(O)ccc(-c2n[nH]c(=S)n2-c2cccc3ccccc23)c1O] | 3-(2,4-Dihydroxy-3-propyl- phenyl)-4-(naphthalen-1-yl)- 5-mercapto-[1,2,4] triazole |
| 59 | 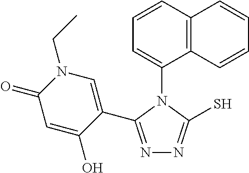 [SMILES: CCn1cc(-c2nnc(S)n2-c2cccc3ccccc23)c(O)cc1=O] | 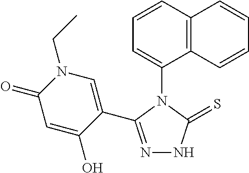 [SMILES: CCn1cc(-c2n[nH]c(=S)n2-c2cccc3ccccc23)c(O)cc1=O] | 3-(1-ethyl-4-hydroxy-6-oxo- 1,6-dihydro-pyridin-3-yl)-4- (naphthalen-1-yl)-5- mercapto-[1,2,4] triazole |
| 60 | 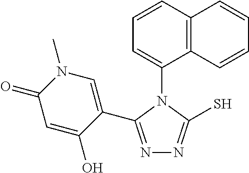 [SMILES: Cn1cc(-c2nnc(S)n2-c2cccc3ccccc23)c(O)cc1=O] | 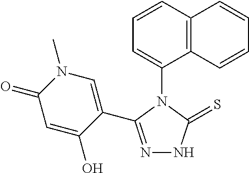 [SMILES: Cn1cc(-c2n[nH]c(=S)n2-c2cccc3ccccc23)c(O)cc1=O] | 3-(4-hydroxy-6-oxo-pyridin- 3-yl)-4-(naphthalen-1-yl)-5- mercapto-[1,2,4] triazole |
| 61 | 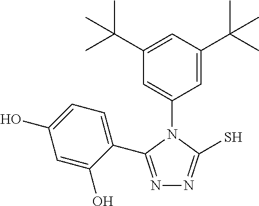 [SMILES: CC(C)(C)c1cc(-n2c(S)nnc2-c2ccc(O)cc2O)cc(C(C)(C)C)c1] | 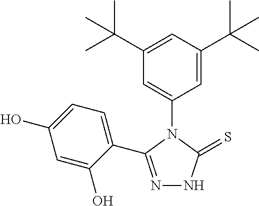 [SMILES: CC(C)(C)c1cc(-n2c(-c3ccc(O)cc3O)n[nH]c2=S)cc(C(C)(C)C)c1] | 3-(2,4-Dihydroxy-phenyl)-4- (3,5-di-tert-butylphenyl)-5- mercapto-[1,2,4] triazole |
| 62 | 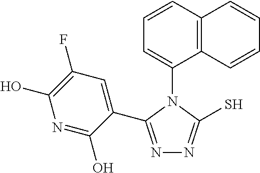 [SMILES: Oc1nc(O)c(-c2nnc(S)n2-c2cccc3ccccc23)cc1F] | 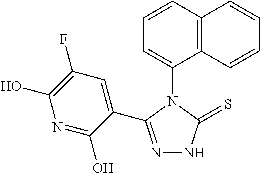 [SMILES: Oc1nc(O)c(-c2n[nH]c(=S)n2-c2cccc3ccccc23)cc1F] | 3-(2,6-Dihydroxy5-fluoro- pyridin-3-yl) 4-(naphthalen- 1-yl)-5-mercapto-[1,2,4] triazole |
| 63 | 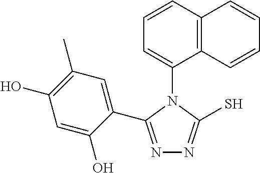 [SMILES: Cc1cc(-c2nnc(S)n2-c2cccc3ccccc23)c(O)cc1O] | 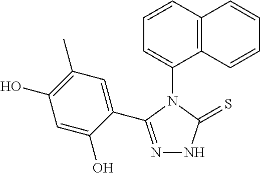 [SMILES: Cc1cc(-c2n[nH]c(=S)n2-c2cccc3ccccc23)c(O)cc1O] | 3-(2,4-Dihydroxy-5-methyl- phenyl)-4-(naphthalene-1-yl)- 5-mercapto-[1,2,4] triazole |
| 64 | 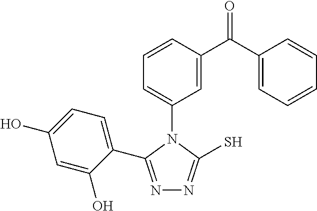 [SMILES: O=C(c1ccccc1)c1cccc(-n2c(S)nnc2-c2ccc(O)cc2O)c1] | 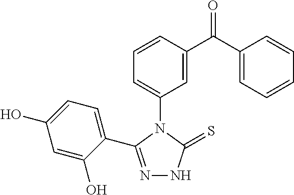 [SMILES: O=C(c1ccccc1)c1cccc(-n2c(-c3ccc(O)cc3O)n[nH]c2=S)c1] | 3-[2,4-Dihydroxy-phenyl]-4- (3-benzoylphenyl)-5- mercapto-[1,2,4] triazole |
| 65 | 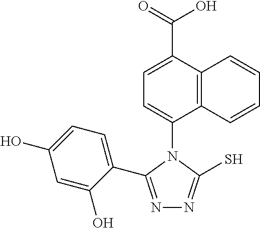 [SMILES: O=C(O)c1ccc(-n2c(S)nnc2-c2ccc(O)cc2O)c2ccccc12] | 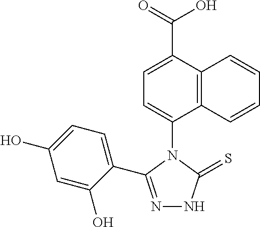 [SMILES: O=C(O)c1ccc(-n2c(-c3ccc(O)cc3O)n[nH]c2=S)c2ccccc12] | 3-(2,4-Dihydroxy-phenyl)-4- (4-carboxy-naphthalen-1-yl)- 5-mercapto-[1,2,4] triazole |
| 66 | 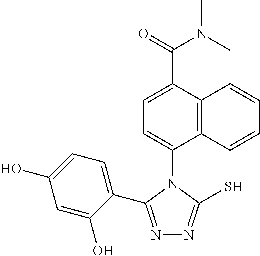 [SMILES: CN(C)C(=O)c1ccc(-n2c(S)nnc2-c2ccc(O)cc2O)c2ccccc12] | 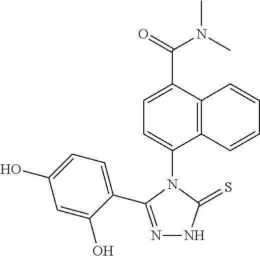 [SMILES: CN(C)C(=O)c1ccc(-n2c(-c3ccc(O)cc3O)n[nH]c2=S)c2ccccc12] | 3-(2,4-Dihydroxy-phenyl)-4- [4-(N,N-dimethylcarbamoyl)- naphthalen-1-yl]-5-mercapto- [1,2,4] triazole |
| 67 | 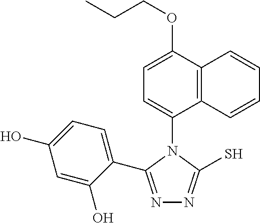 [SMILES: CCCOc1ccc(-n2c(S)nnc2-c2ccc(O)cc2O)c2ccccc12] | 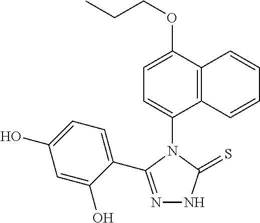 [SMILES: CCCOc1ccc(-n2c(-c3ccc(O)cc3O)n[nH]c2=S)c2ccccc12] | 3-(2,4-Dihydroxy-phenyl)-4- (4-propoxy-naphthalen-1-yl)- 5-mercapto-[1,2,4] triazole |
| 68 | 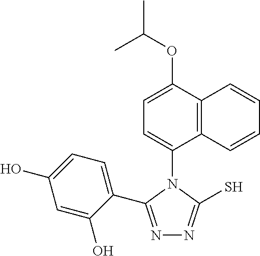 [SMILES: CC(C)Oc1ccc(-n2c(S)nnc2-c2ccc(O)cc2O)c2ccccc12] | 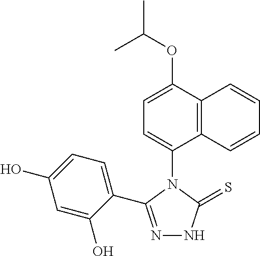 [SMILES: CC(C)Oc1ccc(-n2c(-c3ccc(O)cc3O)n[nH]c2=S)c2ccccc12] | 3-(2,4-Dihydroxy-phenyl)-4- (4-isopropoxy-naphthalen-1- yl)-5-mercapto-[1,2,4] triazole |
| 69 | 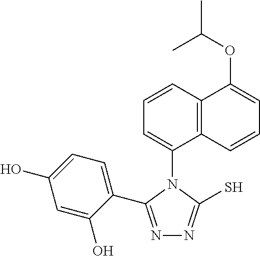 [SMILES: CC(C)Oc1cccc2c(-n3c(S)nnc3-c3ccc(O)cc3O)cccc12] | 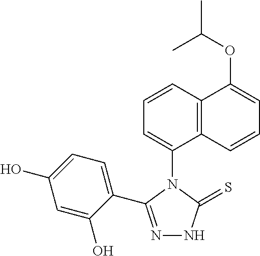 [SMILES: CC(C)Oc1cccc2c(-n3c(-c4ccc(O)cc4O)n[nH]c3=S)cccc12] | 3-(2,4-Dihydroxy-phenyl)-4- (5-isopropoxy-naphthalen-1- yl)-5-mercapto-[1,2,4] triazole |
| 70 | 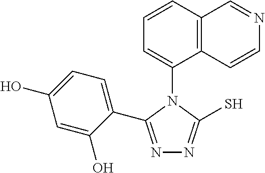 [SMILES: Oc1ccc(-c2nnc(S)n2-c2cccc3cnccc23)c(O)c1] | 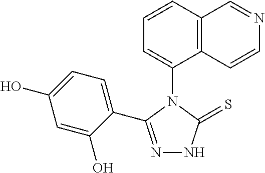 [SMILES: Oc1ccc(-c2n[nH]c(=S)n2-c2cccc3cnccc23)c(O)c1] | 3-(2,4-Dihydroxy-phenyl)-4- (isoquinolin-5-yl)-5- mercapto-[1,2,4] triazole |
| 71 | 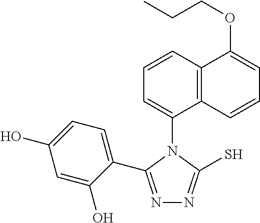 [SMILES: CCCOc1cccc2c(-n3c(S)nnc3-c3ccc(O)cc3O)cccc12] | 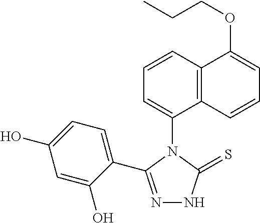 [SMILES: CCCOc1cccc2c(-n3c(-c4ccc(O)cc4O)n[nH]c3=S)cccc12] | 3-(2,4-Dihydroxy-phenyl)-4- (5-propoxy-naphthalen-1-yl)- 5-mercapto-[1,2,4] triazole |
| 72 | 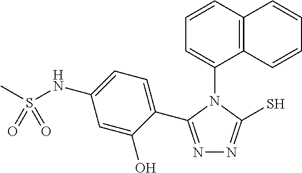 [SMILES: CS(=O)(=O)Nc1ccc(-c2nnc(S)n2-c2cccc3ccccc23)c(O)c1] | 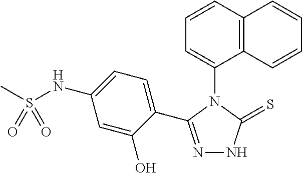 [SMILES: CS(=O)(=O)Nc1ccc(-c2n[nH]c(=S)n2-c2cccc3ccccc23)c(O)c1] | 3-(2-Hydroxy-4- methanesulfonamino- phenyl)-4-(naphthalen-1-yl)- 5-mercapto-[1,2,4] triazole |
| 73 | 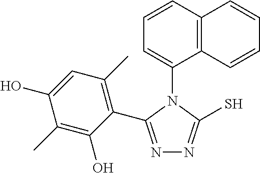 [SMILES: Cc1cc(O)c(C)c(O)c1-c1nnc(S)n1-c1cccc2ccccc12] | 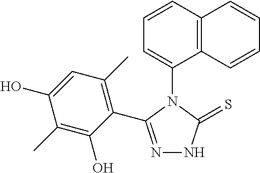 [SMILES: Cc1cc(O)c(C)c(O)c1-c1n[nH]c(=S)n1-c1cccc2ccccc12] | 3-(2,4-Dihydroxy-3,6- dimethyl-phenyl)-4- (naphthalen-1-yl)-5- mercapto-[1,2,4] triazole |
| 74 | 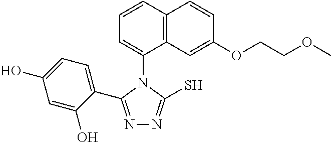 [SMILES: COCCOc1ccc2cccc(-n3c(S)nnc3-c3ccc(O)cc3O)c2c1] | 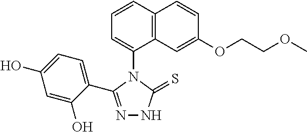 [SMILES: COCCOc1ccc2cccc(-n3c(-c4ccc(O)cc4O)n[nH]c3=S)c2c1] | 3-(2,4-Dihydroxy-phenyl)-4- [7-(2-methoxyethoxy)- napthalen-1-yl]-5-mercapto- [1,2,4] triazole |
| 75 | 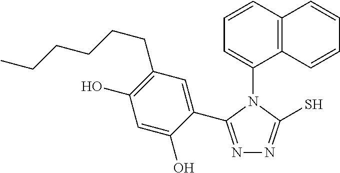 [SMILES: CCCCCCc1cc(-c2nnc(S)n2-c2cccc3ccccc23)c(O)cc1O] | 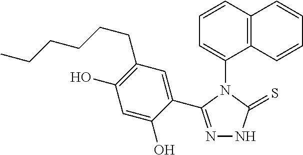 [SMILES: CCCCCCc1cc(-c2n[nH]c(=S)n2-c2cccc3ccccc23)c(O)cc1O] | 3-(2,4-Dihydroxy-5-hexyl- phenyl)-4-(naphthalen-1-yl)- 5-mercapto-[1,2,4] triazole |
| 76 | 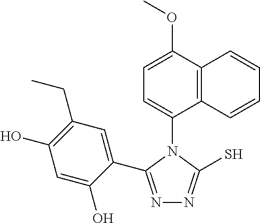 [SMILES: CCc1cc(-c2nnc(S)n2-c2ccc(OC)c3ccccc23)c(O)cc1O] | 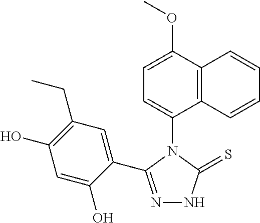 [SMILES: CCc1cc(-c2n[nH]c(=S)n2-c2ccc(OC)c3ccccc23)c(O)cc1O] | 3-(2,4-Dihydroxy-5-ethyl- phenyl)-4-(4-methoxy- naphthalen-1-yl)-5-mercapto- [1,2,4] triazole |
| 77 | 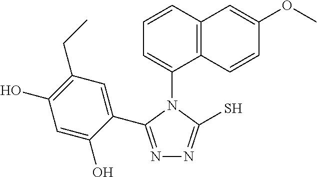 [SMILES: CCc1cc(-c2nnc(S)n2-c2cccc3cc(OC)ccc23)c(O)cc1O] | 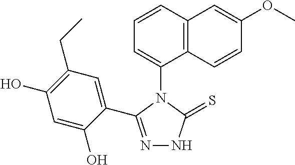 [SMILES: CCc1cc(-c2n[nH]c(=S)n2-c2cccc3cc(OC)ccc23)c(O)cc1O] | 3-(2,4-Dihydroxy-5-ethyl- phenyl)-4-(6-methoxy- naphthalin-1-yl)-5-mercapto- [1,2,4] triazole |
| 78 | 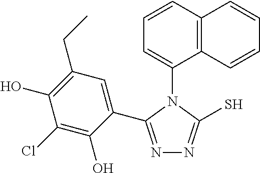 [SMILES: CCc1cc(-c2nnc(S)n2-c2cccc3ccccc23)c(O)c(Cl)c1O] | 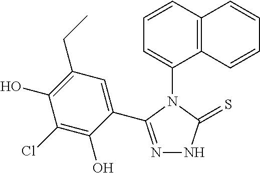 [SMILES: CCc1cc(-c2n[nH]c(=S)n2-c2cccc3ccccc23)c(O)c(Cl)c1O] | 3-(2,4-Dihydroxy-3-chloro-5- ethyl-phenyl)-4-(naphthalen- 1-yl)-5-mercapto-[1,2,4] triazole |
| 79 | 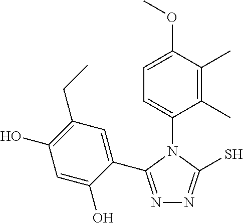 [SMILES: CCc1cc(-c2nnc(S)n2-c2ccc(OC)c(C)c2C)c(O)cc1O] | 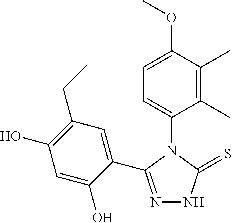 [SMILES: CCc1cc(-c2n[nH]c(=S)n2-c2ccc(OC)c(C)c2C)c(O)cc1O] | 3-(2,4-Dihydroxy-5-ethyl- phenyl)-4-(2,3-dimethy-4- methoxy-phenyl)-5- mercapto-[1,2,4] triazole |
| 80 | 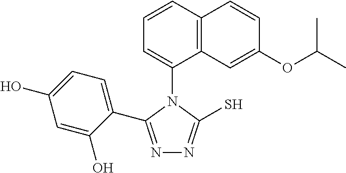 [SMILES: CC(C)Oc1ccc2cccc(-n3c(S)nnc3-c3ccc(O)cc3O)c2c1] | 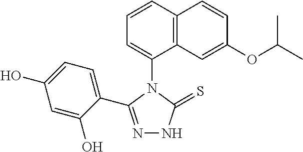 [SMILES: CC(C)Oc1ccc2cccc(-n3c(-c4ccc(O)cc4O)n[nH]c3=S)c2c1] | 3-(2,4-Dihydroxy-phenyl)-4- (7-isopropoxy-naphthalen-1- yl)-5-mercapto-[1,2,4] triazole |
| 81 | 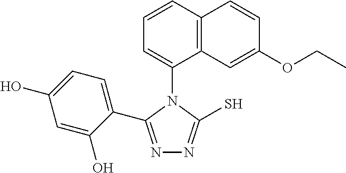 [SMILES: CCOc1ccc2cccc(-n3c(S)nnc3-c3ccc(O)cc3O)c2c1] | 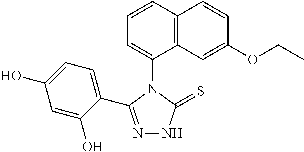 [SMILES: CCOc1ccc2cccc(-n3c(-c4ccc(O)cc4O)n[nH]c3=S)c2c1] | 3-(2,4-Dihydroxy-phenyl)-4- (7-ethoxy-naphthalen-1-yl)- 5-mercapto-[1,2,4] triazole |
| 82 | 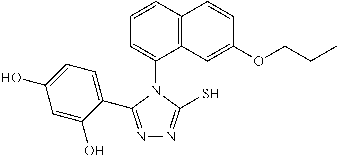 [SMILES: CCCOc1ccc2cccc(-n3c(S)nnc3-c3ccc(O)cc3O)c2c1] | 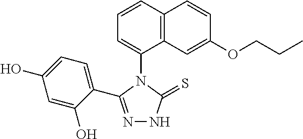 [SMILES: CCCOc1ccc2cccc(-n3c(-c4ccc(O)cc4O)n[nH]c3=S)c2c1] | 3-(2,4-Dihydroxy-phenyl)-4- (7-propoxy-naphthalen-1-yl)- 5-mercapto-[1,2,4] triazole |
| 83 | 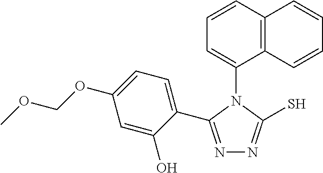 [SMILES: COCOc1ccc(-c2nnc(S)n2-c2cccc3ccccc23)c(O)c1] | 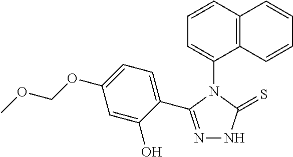 [SMILES: COCOc1ccc(-c2n[nH]c(=S)n2-c2cccc3ccccc23)c(O)c1] | 3-(2-Hydroxy-4- methoxymethyoxy-phenyl)- 4-(naphthalen-1-yl)-5- mercapto-[1,2,4] triazole |
| 84 | 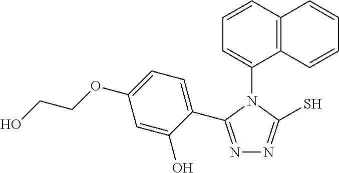 [SMILES: OCCOc1ccc(-c2nnc(S)n2-c2cccc3ccccc23)c(O)c1] | 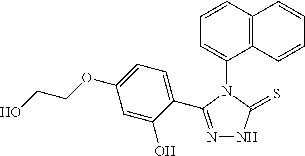 [SMILES: OCCOc1ccc(-c2n[nH]c(=S)n2-c2cccc3ccccc23)c(O)c1] | 3-[2-Hydroxy-4-(2-hydroxy- ethoxy)-phenyl]-4- (naphthalen-1-yl)-5- mercapto-[1,2,4] triazole |
| 85 | 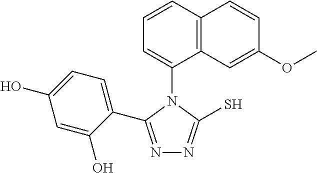 [SMILES: COc1ccc2cccc(-n3c(S)nnc3-c3ccc(O)cc3O)c2c1] | 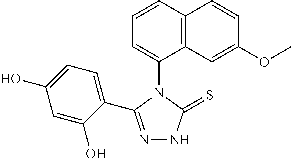 [SMILES: COc1ccc2cccc(-n3c(-c4ccc(O)cc4O)n[nH]c3=S)c2c1] | 3-(2,4-Dihydroxyphenyl)-4- (7-methoxy-naphthalen-1-yl)- 5-mercapto-[1,2,4] triazole |
| 86 | 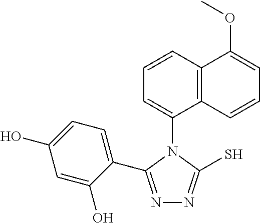 [SMILES: COc1cccc2c(-n3c(S)nnc3-c3ccc(O)cc3O)cccc12] | 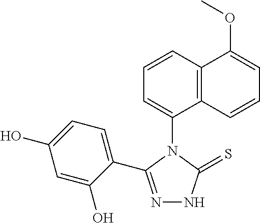 [SMILES: COc1cccc2c(-n3c(-c4ccc(O)cc4O)n[nH]c3=S)cccc12] | 3-(2,4-Dihydroxyphenyl)-4- (5-methoxy-naphthalen-1-y1)- 5-mercapto-[1,2,4] triazole |
| 87 | 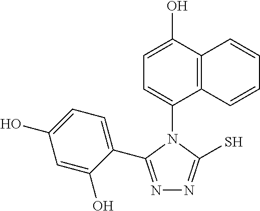 [SMILES: Oc1ccc(-c2nnc(S)n2-c2ccc(O)c3ccccc23)c(O)c1] | 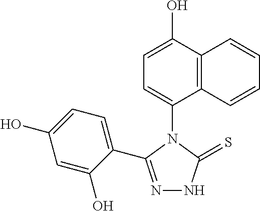 [SMILES: Oc1ccc(-c2n[nH]c(=S)n2-c2ccc(O)c3ccccc23)c(O)c1] | 3-(2,4-Dihydroxyphenyl)-4- (4-hydroxy-naphthalen-1-yl)- 5-mercapto-[1,2,4] triazole |
| 88 | 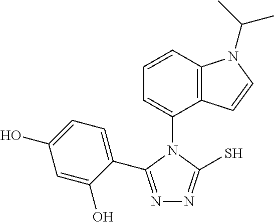 [SMILES: CC(C)n1ccc2c(-n3c(S)nnc3-c3ccc(O)cc3O)cccc21] | 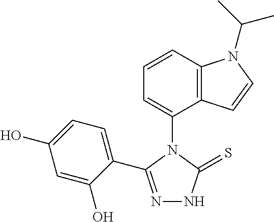 [SMILES: CC(C)n1ccc2c(-n3c(-c4ccc(O)cc4O)n[nH]c3=S)cccc21] | 3-(2,4-Dihydroxyphenyl)-4- (1-isopropyl-indol-4-yl)-5- mercapto-[1,2,4] triazole |
| 89 | 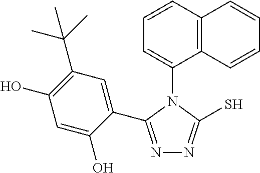 [SMILES: CC(C)(C)c1cc(-c2nnc(S)n2-c2cccc3ccccc23)c(O)cc1O] | 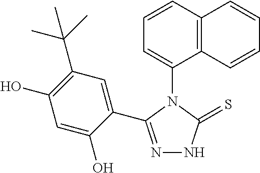 [SMILES: CC(C)(C)c1cc(-c2n[nH]c(=S)n2-c2cccc3ccccc23)c(O)cc1O] | 3-(2,4-Dihydroxy-5-tert- butyl-phenyl)-4-(naphthalen- 1-yl)-5-mercapto-[1,2,4] triazole |
| 90 | 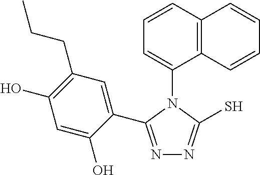 [SMILES: CCCc1cc(-c2nnc(S)n2-c2cccc3ccccc23)c(O)cc1O] | 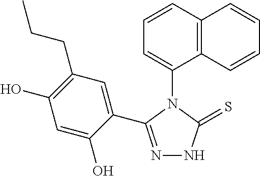 [SMILES: CCCc1cc(-c2n[nH]c(=S)n2-c2cccc3ccccc23)c(O)cc1O] | 3-(2,4-Dihydroxy-5-propyl- phenyl)-4-(naphthalen-1-yl)- 5-mercapto-[1,2,4] triazole |
| 91 | 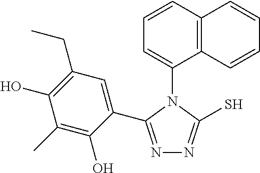 [SMILES: CCc1cc(-c2nnc(S)n2-c2cccc3ccccc23)c(O)c(C)c1O] | 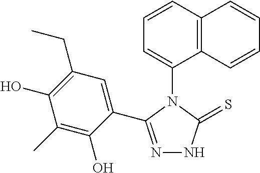 [SMILES: CCc1cc(-c2n[nH]c(=S)n2-c2cccc3ccccc23)c(O)c(C)c1O] | 3-(2,4-Dihydroxy-3-methyl- 5-ethyl-phenyl)-4- (naphthalen-1-yl)-5- mercapto-[1,2,4] triazole |
| 92 | 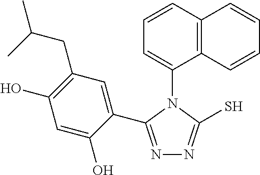 [SMILES: CC(C)Cc1cc(-c2nnc(S)n2-c2cccc3ccccc23)c(O)cc1O] | 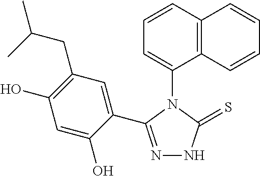 [SMILES: CC(C)Cc1cc(-c2n[nH]c(=S)n2-c2cccc3ccccc23)c(O)cc1O] | 3-(2,4-Dihydroxy-5-isobutyl- phenyl)-4-(naphthalen-1-yl)- 5-mercapto-[1,2,4] triazole |
| 93 | 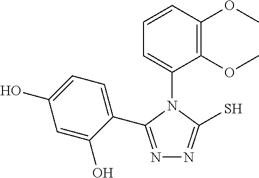 [SMILES: COc1cccc(-n2c(S)nnc2-c2ccc(O)cc2O)c1OC] | 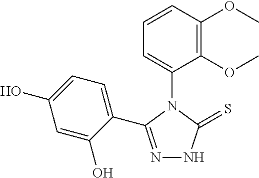 [SMILES: COc1cccc(-n2c(-c3ccc(O)cc3O)n[nH]c2=S)c1OC] | 3-(2,4-Dihydroxy-phenyl)-4- (2,3-dimethoxy-phenyl)-5- mercapto-[1,2,4] triazole |
| 94 | 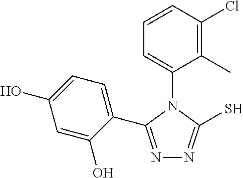 [SMILES: Cc1c(Cl)cccc1-n1c(S)nnc1-c1ccc(O)cc1O] | 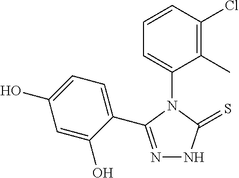 [SMILES: Cc1c(Cl)cccc1-n1c(-c2ccc(O)cc2O)n[nH]c1=S] | 3-(2,4-Dihydroxy-phenyl)-4- (2-methoxy-3-chloro- phenyl)-5-mercapto-[1,2,4] triazole |
| 95 | 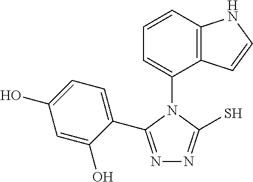 [SMILES: Oc1ccc(-c2nnc(S)n2-c2cccc3[nH]ccc23)c(O)c1] | 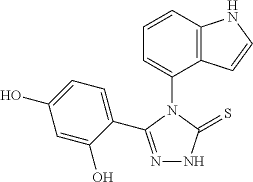 [SMILES: Oc1ccc(-c2n[nH]c(=S)n2-c2cccc3[nH]ccc23)c(O)c1] | 3-(2,4-Dihydroxy-phenyl)-4- (indol-4-yl)-5-mercapto- [1,2,4] triazole |
| 96 | 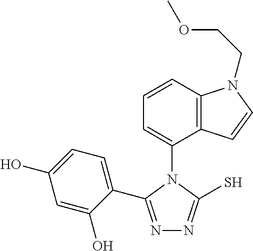 [SMILES: COCCn1ccc2c(-n3c(S)nnc3-c3ccc(O)cc3O)cccc21] | 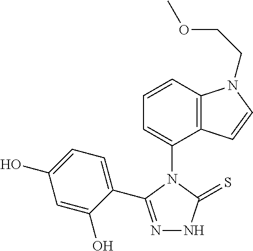 [SMILES: COCCn1ccc2c(-n3c(-c4ccc(O)cc4O)n[nH]c3=S)cccc21] | 3-(2,4-Dihydroxy-phenyl)-4- [1-(2-methoxyethoxy)-indol- 4-yl]-5-mercapto-[1,2,4] triazole |
| 97 | 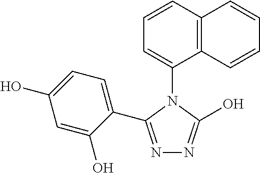 [SMILES: Oc1ccc(-c2nnc(O)n2-c2cccc3ccccc23)c(O)c1] | 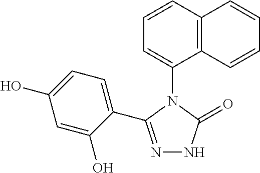 [SMILES: O=c1[nH]nc(-c2ccc(O)cc2O)n1-c1cccc2ccccc12] | 3-(2,4-Dihydroxy-phenyl)-4- (naphthalen-1-yl)-5-hydroxy- [1,2,4] triazole |
| 98 | 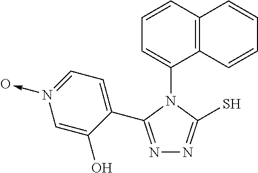 [SMILES: [O-][n+]1ccc(-c2nnc(S)n2-c2cccc3ccccc23)c(O)c1] | 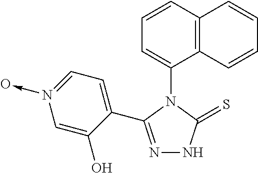 [SMILES: [O-][n+]1ccc(-c2n[nH]c(=S)n2-c2cccc3ccccc23)c(O)c1] | 3-(1-Oxo-3-hydroxy-pyridin- 4-yl)-4-(naphthalen-1-yl)-5- mercapto-[1,2,4] triazole |
| 99 | 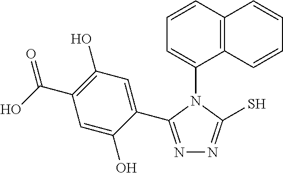 [SMILES: O=C(O)c1cc(O)c(-c2nnc(S)n2-c2cccc3ccccc23)cc1O] | 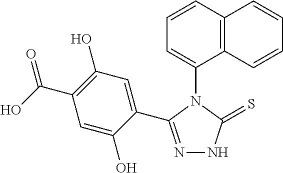 [SMILES: O=C(O)c1cc(O)c(-c2n[nH]c(=S)n2-c2cccc3ccccc23)cc1O] | 3-(2,5-Dihydroxy-4- carboxy)-4-(naphthalen-1- yl)-5-mercapto-[1,2,4] triazole |
| 100 | 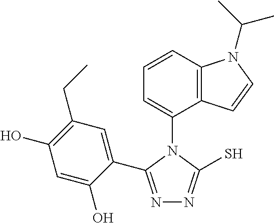 [SMILES: CCc1cc(-c2nnc(S)n2-c2cccc3c2ccn3C(C)C)c(O)cc1O] | 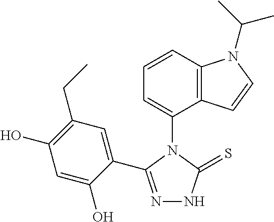 [SMILES: CCc1cc(-c2n[nH]c(=S)n2-c2cccc3c2ccn3C(C)C)c(O)cc1O] | 3-(2,4-Dihydroxy-5-ethyl- phenyl)-4-(1-isopropyl-indol- 4-yl)-5-mercapto-[1,2,4] triazole |
| 101 | 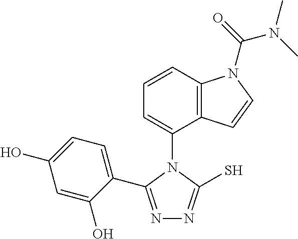 [SMILES: CN(C)C(=O)n1ccc2c(-n3c(S)nnc3-c3ccc(O)cc3O)cccc21] | 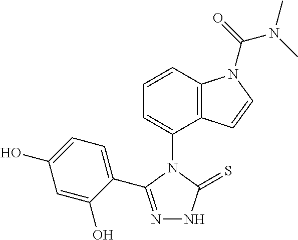 [SMILES: CN(C)C(=O)n1ccc2c(-n3c(-c4ccc(O)cc4O)n[nH]c3=S)cccc21] | 3-(2,4-Dihydroxy-5-ethyl- phenyl)-4-[1-(dimethyl- carbamoyl)-indol-4-yl]-5- mercapto-[1,2,4] triazole |
| 102 | 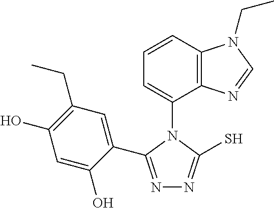 [SMILES: CCc1cc(-c2nnc(S)n2-c2cccc3c2ncn3CC)c(O)cc1O] | 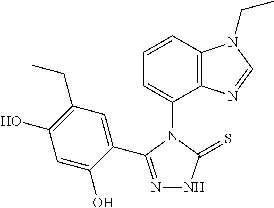 [SMILES: CCc1cc(-c2n[nH]c(=S)n2-c2cccc3c2ncn3CC)c(O)cc1O] | 3-(2,4-Dihydroxy-5-ethyl- phenyl)-4-(1-ethyl- benzoimidazol-4-yl)-5- mercapto-[1,2,4] triazole |
| 103 | 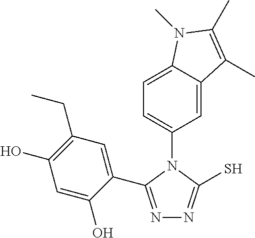 [SMILES: CCc1cc(-c2nnc(S)n2-c2ccc3c(c2)c(C)c(C)n3C)c(O)cc1O] | 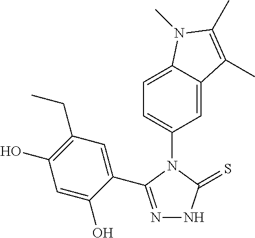 [SMILES: CCc1cc(-c2n[nH]c(=S)n2-c2ccc3c(c2)c(C)c(C)n3C)c(O)cc1O] | 3-(2,4-Dihydroxy-5-ethyl- phenyl)-4-(1,2,3-trimethyl- indol-5-yl)-5-mercapto- [1,2,4] triazole |
| 104 | 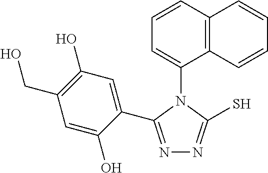 [SMILES: OCc1cc(O)c(-c2nnc(S)n2-c2cccc3ccccc23)cc1O] | 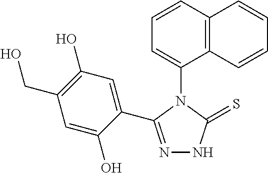 [SMILES: OCc1cc(O)c(-c2n[nH]c(=S)n2-c2cccc3ccccc23)cc1O] | 3-(2,5-Dihydroxy-4- hydroxymethyl-phenyl)-4- (naphthalen-1-yl)-5- mercapto-[1,2,4] triazole |
| 105 | 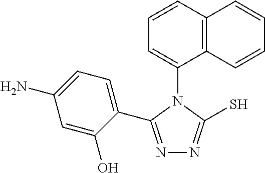 [SMILES: Nc1ccc(-c2nnc(S)n2-c2cccc3ccccc23)c(O)c1] | 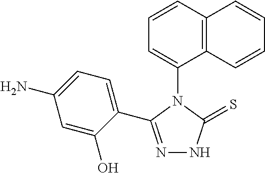 [SMILES: Nc1ccc(-c2n[nH]c(=S)n2-c2cccc3ccccc23)c(O)c1] | 3-(2-Hydroxy-4-amino- phenyl)-4-(naphthalen-1-yl)- 5-mercapto-[1,2,4] triazole |
| 106 | 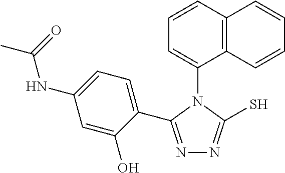 [SMILES: CC(=O)Nc1ccc(-c2nnc(S)n2-c2cccc3ccccc23)c(O)c1] | 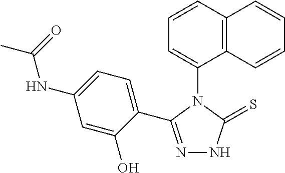 [SMILES: CC(=O)Nc1ccc(-c2n[nH]c(=S)n2-c2cccc3ccccc23)c(O)c1] | 3-(2-Hydroxy-4-acetylamino- phenyl)-4-(naphthalen-1-yl)- 5-mercapto-[1,2,4] triazole |
| 107 | 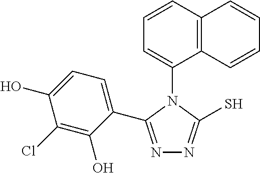 [SMILES: Oc1ccc(-c2nnc(S)n2-c2cccc3ccccc23)c(O)c1Cl] | 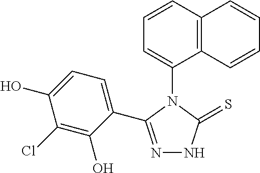 [SMILES: Oc1ccc(-c2n[nH]c(=S)n2-c2cccc3ccccc23)c(O)c1Cl] | 3-(2,4-Dihydroxy-3-chloro- phenyl)-4-(naphthalen-1-yl)- 5-mercapto-[1,2,4] triazole |
| 108 | 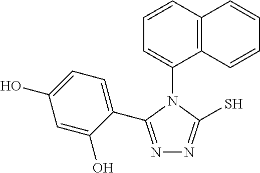 [SMILES: Oc1ccc(-c2nnc(S)n2-c2cccc3ccccc23)c(O)c1] | 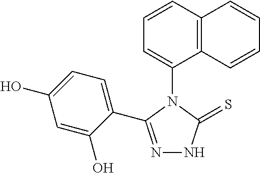 [SMILES: Oc1ccc(-c2n[nH]c(=S)n2-c2cccc3ccccc23)c(O)c1] | 3-(2,4-Dihydroxy-phenyl)-4- (naphthalen-1-yl)-5- mercapto-[1,2,4] triazole |
| 109 | 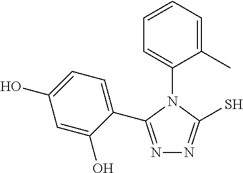 [SMILES: Cc1ccccc1-n1c(S)nnc1-c1ccc(O)cc1O] | 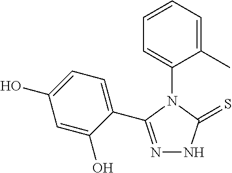 [SMILES: Cc1ccccc1-n1c(-c2ccc(O)cc2O)n[nH]c1=S] | 3-(2,4-Dihydroxy-phenyl)-4- (2-methyl-phenyl)-5- mercapto-[1,2,4] triazole |
| 110 | 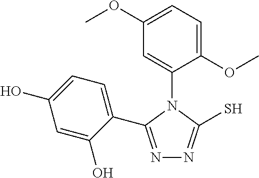 [SMILES: COc1ccc(OC)c(-n2c(S)nnc2-c2ccc(O)cc2O)c1] | 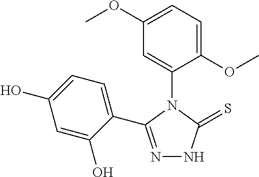 [SMILES: COc1ccc(OC)c(-n2c(-c3ccc(O)cc3O)n[nH]c2=S)c1] | 3-(2,4-Dihydroxy-phenyl)-4- (2,5-dimethoxy-phenyl)-5- mercapto-[1,2,4] triazole |
| 111 | 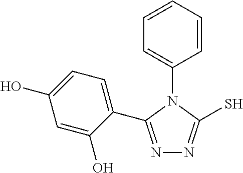 [SMILES: Oc1ccc(-c2nnc(S)n2-c2ccccc2)c(O)c1] | 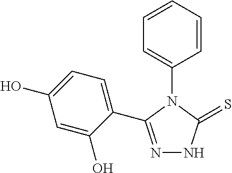 [SMILES: Oc1ccc(-c2n[nH]c(=S)n2-c2ccccc2)c(O)c1] | 3-(2,4-Dihydroxy-phenyl)-4- phenyl-5-mercapto-[1,2,4] triazole |
| 112 | 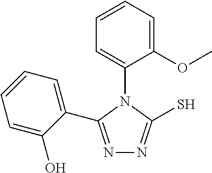 [SMILES: COc1ccccc1-n1c(S)nnc1-c1ccccc1O] | 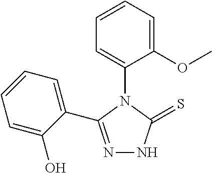 [SMILES: COc1ccccc1-n1c(-c2ccccc2O)n[nH]c1=S] | 3-(2-Hydroxy-phenyl)-4-(2- methoxy-phenyl)-5- mercapto-[1,2,4] triazole |
| 113 | 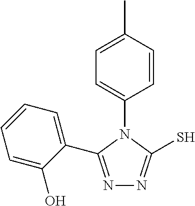 [SMILES: Cc1ccc(-n2c(S)nnc2-c2ccccc2O)cc1] | 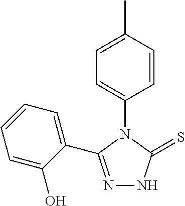 [SMILES: Cc1ccc(-n2c(-c3ccccc3O)n[nH]c2=S)cc1] | 3-(2-Hydroxy-phenyl)-4-(4- methyl-phenyl)-5-mercapto- [1,2,4] triazole |
| 114 | 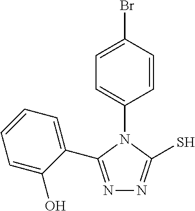 [SMILES: Oc1ccccc1-c1nnc(S)n1-c1ccc(Br)cc1] | 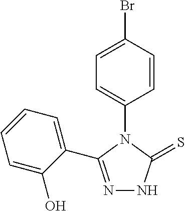 [SMILES: Oc1ccccc1-c1n[nH]c(=S)n1-c1ccc(Br)cc1] | 3-(2-Hydroxy-phenyl)-4-(4- bromo-phenyl)-5-mercapto- [1,2,4] triazole |
| 115 | 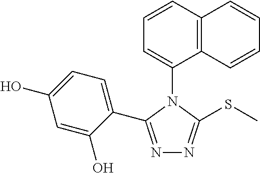 [SMILES: CSc1nnc(-c2ccc(O)cc2O)n1-c1cccc2ccccc12] | | 3-(2,4-Dihydroxy-phenyl)-4- (naphthalen-1-yl)-5-(methyl sulfanyl)-[1,2,4] triazole |
| 116 | 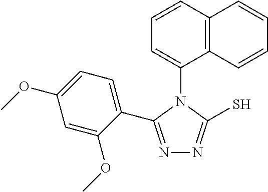 [SMILES: COc1ccc(-c2nnc(S)n2-c2cccc3ccccc23)c(OC)c1] | 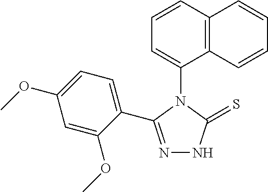 [SMILES: COc1ccc(-c2n[nH]c(=S)n2-c2cccc3ccccc23)c(OC)c1] | 3-(2,4-Dimethoxy-phenyl)-4- (naphthalen-1-yl)-5- mercapto-[1,2,4] triazole |
| 117 | 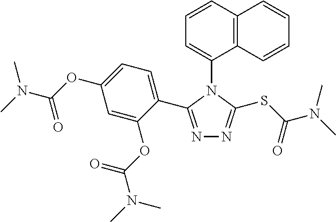 | | 3-[2,4-Di-(dimethyl- carbamoyloxy)-phenyl]-4- (naphthalen-1-yl)-5- (dimethyl- carbamoylsulfanyl)-[1,2,4] triazole |
| 118 | 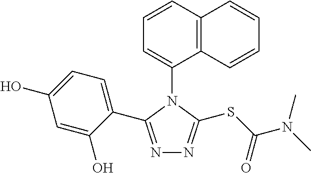 [SMILES: CN(C)C(=O)Sc1nnc(-c2ccc(O)cc2O)n1-c1cccc2ccccc12] | | 3-(2,4-Dihydroxy-phenyl)-4- (naphthalen-1-yl)-5- (dimethylcarbamoylsulfanyl)- [1,2,4] triazole |
| 119 | 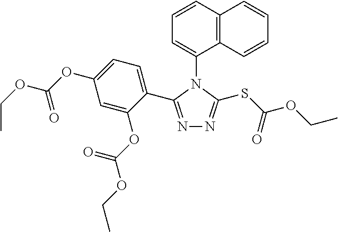 [SMILES: CCOC(=O)Oc1ccc(-c2nnc(SC(=O)OCC)n2-c2cccc3ccccc23)c(OC(=O)OCC)c1] | | 3-(2,4-Diethoxycarbonyloxy- phenyl)-4-(naphthalen-1-yl)- 5-(ethoxycarbonylsulfanyl)- [1,2,4] triazole |
| 120 | 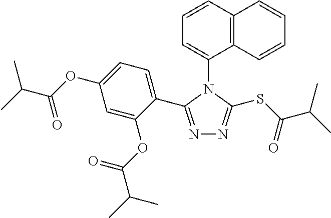 [SMILES: CC(C)C(=O)Oc1ccc(-c2nnc(SC(=O)C(C)C)n2-c2cccc3ccccc23)c(OC(=O)C(C)C)c1] | | 3-(2,4-Di-isobutyryloxy- phenyl)-4-(naphthalen-1-yl)- 5-(isobutyrylsulfanyl)-[1,2,4] triazole |
| 121 | 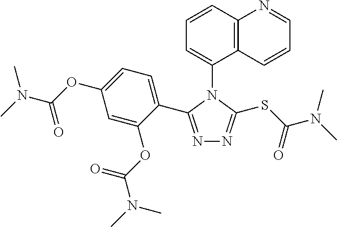 | | 3-[2,4-Di-(dimethyl- carbamoyloxy)-phenyl]-4- (quinolin-5-yl)-5-(dimethyl- carbamoylsulfanyl)-[1,2,4] triazole |
| 122 | 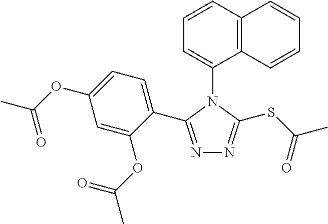 [SMILES: CC(=O)Oc1ccc(-c2nnc(SC(C)=O)n2-c2cccc3ccccc23)c(OC(C)=O)c1] | | 3-(2,4-Diacetoxy-phenyl)-4- (naphthalen-1-yl)-5- (acetylsulfanyl)-[1,2,4] triazole |
| 123 | 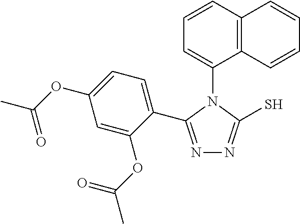 [SMILES: CC(=O)Oc1ccc(-c2nnc(S)n2-c2cccc3ccccc23)c(OC(C)=O)c1] | 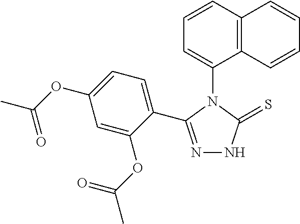 [SMILES: CC(=O)Oc1ccc(-c2n[nH]c(=S)n2-c2cccc3ccccc23)c(OC(C)=O)c1] | 3-(2,4-Diacetoxy-phenyl)-4- (naphthalen-1-yl)-5- mercapto-[1,2,4] triazole |
| 124 | 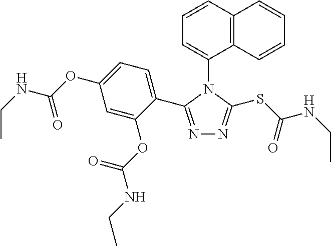 [SMILES: CCNC(=O)Oc1ccc(-c2nnc(SC(=O)NCC)n2-c2cccc3ccccc23)c(OC(=O)NCC)c1] | | 3-(2,4-Diethylcarbamoyloxy- phenyl)-4-(naphthalen-1-yl)- 5-(ethylcarbamoylsulfanyl)- [1,2,4] triazole |
| 125 | 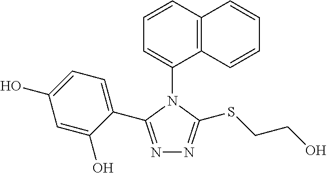 [SMILES: OCCSc1nnc(-c2ccc(O)cc2O)n1-c1cccc2ccccc12] | | 3-(2,4-Dihydroxy-phenyl)-4- (naphthalen-1-yl)-5-(2- hydroxyethylsulfanyl)- [1,2,4] triazole |
| 126 | 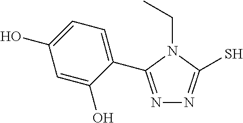 [SMILES: CCn1c(S)nnc1-c1ccc(O)cc1O] | 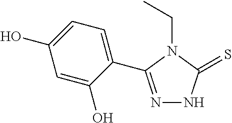 [SMILES: CCn1c(-c2ccc(O)cc2O)n[nH]c1=S] | 3-(2,4-Dihydroxy-phenyl)-4- ethyl-5-mercapto-[1,2,4] triazole |
| 127 | 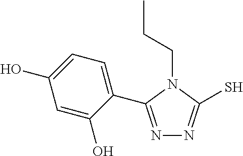 [SMILES: CCCn1c(S)nnc1-c1ccc(O)cc1O] | 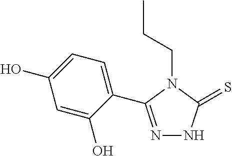 [SMILES: CCCn1c(-c2ccc(O)cc2O)n[nH]c1=S] | 3-(2,4-Dihydroxy-phenyl)-4- propyl-5-mercapto-[1,2,4] triazole |
| 128 | 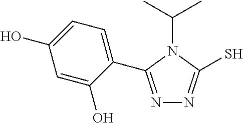 [SMILES: CC(C)n1c(S)nnc1-c1ccc(O)cc1O] | 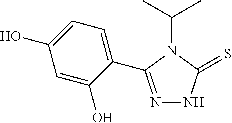 [SMILES: CC(C)n1c(-c2ccc(O)cc2O)n[nH]c1=S] | 3-(2,4-Dihydroxy-phenyl)-4- isopropyl-5-mercapto-[1,2,4] triazole |
| 129 | 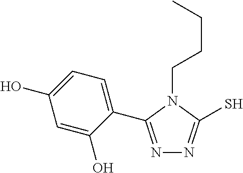 [SMILES: CCCCn1c(S)nnc1-c1ccc(O)cc1O] | 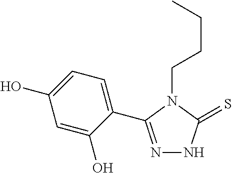 [SMILES: CCCCn1c(-c2ccc(O)cc2O)n[nH]c1=S] | 3-(2,4-Dihydroxy-phenyl)-4- butyl-5-mercapto-[1,2,4] triazole |
| 130 | 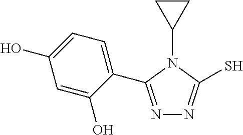 [SMILES: Oc1ccc(-c2nnc(S)n2C2CC2)c(O)c1] | 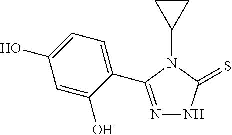 [SMILES: Oc1ccc(-c2n[nH]c(=S)n2C2CC2)c(O)c1] | 3-(2,4-Dihydroxy-phenyl)-4- cyclopropyl-5-mercapto- [1,2,4] triazole |
| 131 | 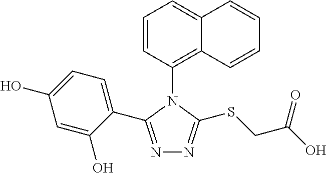 [SMILES: O=C(O)CSc1nnc(-c2ccc(O)cc2O)n1-c1cccc2ccccc12] | | 3-(2,4-Dihydroxy-phenyl)-4- (naphthalen-1-yl)-5- (carboxyethysulfanyl)- [1,2,4] triazole |
| 132 | 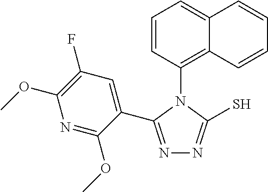 [SMILES: COc1nc(OC)c(-c2nnc(S)n2-c2cccc3ccccc23)cc1F] | 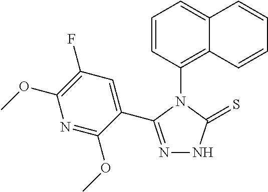 [SMILES: COc1nc(OC)c(-c2n[nH]c(=S)n2-c2cccc3ccccc23)cc1F] | 3-(2,6-Dimethoxy-5-fluoro- pyridin-3-yl)-4-(naphthalen- 1-yl)-5-mercapto-[1,2,4] triazole |
| 133 | 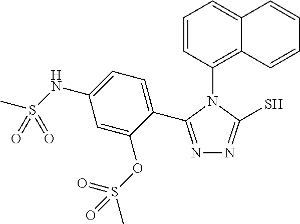 [SMILES: CS(=O)(=O)Nc1ccc(-c2nnc(S)n2-c2cccc3ccccc23)c(OS(C)(=O)=O)c1] | 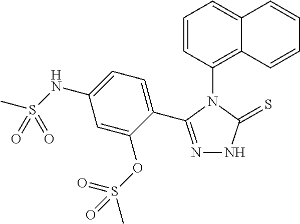 [SMILES: CS(=O)(=O)Nc1ccc(-c2n[nH]c(=S)n2-c2cccc3ccccc23)c(OS(C)(=O)=O)c1] | 3-(2-Methanesulfonyloxy-4- methanesulfonylamino- phenyl)-4-(naphthalen-1-yl)- 5-mercapto-[1,2,4] triazole |
| 134 | 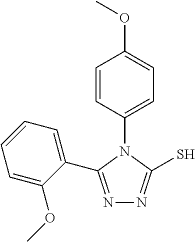 [SMILES: COc1ccc(-n2c(S)nnc2-c2ccccc2OC)cc1] | 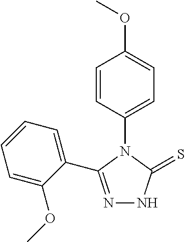 [SMILES: COc1ccc(-n2c(-c3ccccc3OC)n[nH]c2=S)cc1] | 3-(2-Methoxy-phenyl)-4-(4- methoxy-phenyl)-5- mercapto-[1,2,4] triazole |
| 135 | 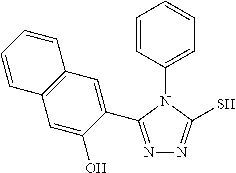 [SMILES: Oc1cc2ccccc2cc1-c1nnc(S)n1-c1ccccc1] | 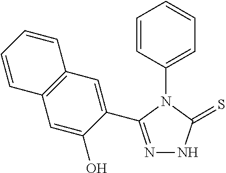 [SMILES: Oc1cc2ccccc2cc1-c1n[nH]c(=S)n1-c1ccccc1] | 3-(3-Hydroxy-naphthalen-2- yl)-4-phenyl-5-mercapto- [1,2,4] triazole |
| 136 | 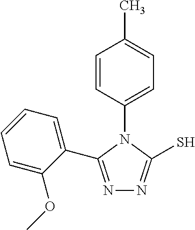 [SMILES: COc1ccccc1-c1nnc(S)n1-c1ccc(C)cc1] | 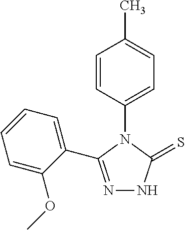 [SMILES: COc1ccccc1-c1n[nH]c(=S)n1-c1ccc(C)cc1] | 3-(2-Methoxy-phenyl)-4-(4- methyl-phenyl)-5-mercapto- [1,2,4] triazole |
| 137 | 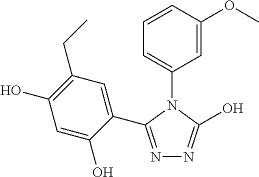 [SMILES: CCc1cc(-c2nnc(O)n2-c2cccc(OC)c2)c(O)cc1O] | 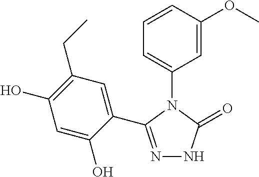 [SMILES: CCc1cc(-c2n[nH]c(=O)n2-c2cccc(OC)c2)c(O)cc1O] | 3-(2,4-Dihydroxy-5-ethyl- phenyl)-4-(3-methox- phenyl)-5-hydroxy-[1,2,4] triazole |
| 138 | 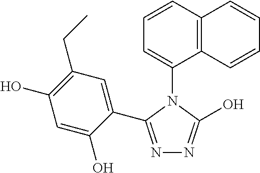 [SMILES: CCc1cc(-c2nnc(O)n2-c2cccc3ccccc23)c(O)cc1O] | 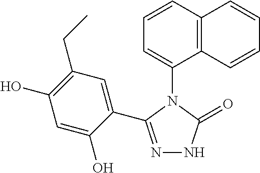 [SMILES: CCc1cc(-c2n[nH]c(=O)n2-c2cccc3ccccc23)c(O)cc1O] | 3-(2,4-Dihydroxy-5-ethyl- phenyl)-4-(naphthalen-1-yl)- 5-hydroxy-[1,2,4] triazole |
| 139 | 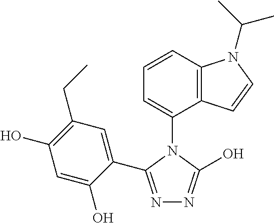 [SMILES: CCc1cc(-c2nnc(O)n2-c2cccc3c2ccn3C(C)C)c(O)cc1O] | 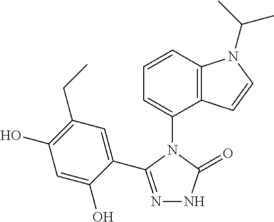 [SMILES: CCc1cc(-c2n[nH]c(=O)n2-c2cccc3c2ccn3C(C)C)c(O)cc1O] | 3-(2,4-Dihydroxy-5-ethyl- phenyl)-4-(1-isopropyl-indol- 3-yl)-5-hydroxy-[1,2,4] triazole |
| 140 | 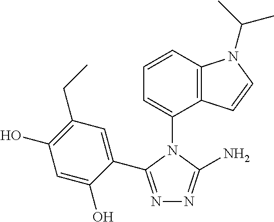 [SMILES: CCc1cc(-c2nnc(N)n2-c2cccc3c2ccn3C(C)C)c(O)cc1O] | 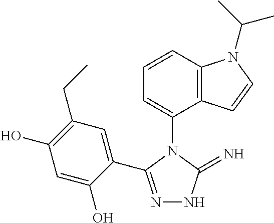 [SMILES: CCc1cc(-c2n[nH]c(=N)n2-c2cccc3c2ccn3C(C)C)c(O)cc1O] | 3-(2,4-Dihydroxy-5-ethyl- phenyl)-4-(1-isopropyl-indol- 4-yl)-5-amino-[1,2,4] triazole |
| 141 | 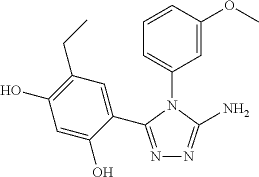 [SMILES: CCc1cc(-c2nnc(N)n2-c2cccc(OC)c2)c(O)cc1O] | 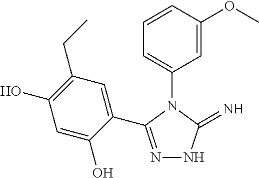 [SMILES: CCc1cc(-c2n[nH]c(=N)n2-c2cccc(OC)c2)c(O)cc1O] | 3-(2,4-Dihydroxy-5-ethyl- phenyl)-4-(3-methoxy- phenyl)-5-amino-[1,2,4] triazole |
| 142 | 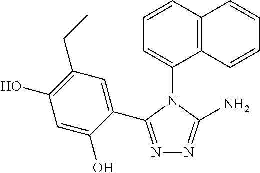 [SMILES: CCc1cc(-c2nnc(N)n2-c2cccc3ccccc23)c(O)cc1O] | 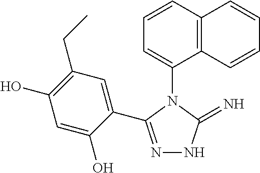 [SMILES: CCc1cc(-c2n[nH]c(=N)n2-c2cccc3ccccc23)c(O)cc1O] | 3-(2,4-Dihydroxy-5-ethyl- phenyl)-4-(naphthalen-1-yl)- 5-amino-[1,2,4] triazole |
| 143 | 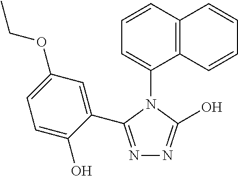 [SMILES: CCOc1ccc(O)c(-c2nnc(O)n2-c2cccc3ccccc23)c1] | 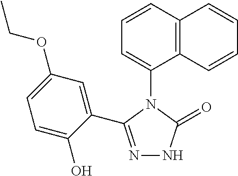 [SMILES: CCOc1ccc(O)c(-c2n[nH]c(=O)n2-c2cccc3ccccc23)c1] | 3-(2-Hydroxy-5-ethyloxy- phenyl)-4-(naphthalen-1-yl)- 5-hydroxy-[1,2,4] triazole |
| 144 | 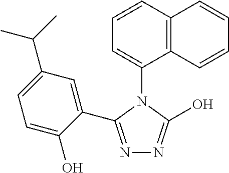 [SMILES: CC(C)c1ccc(O)c(-c2nnc(O)n2-c2cccc3ccccc23)c1] | 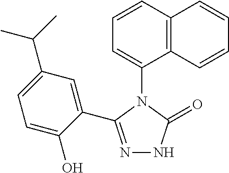 [SMILES: CC(C)c1ccc(O)c(-c2n[nH]c(=O)n2-c2cccc3ccccc23)c1] | 3-(2-Hydroxy-5-isopropyl- phenyl)-4-(naphthalen-1-yl)- 5-hydroxy-[1,2,4] triazole |
| 145 | 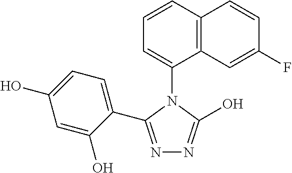 [SMILES: Oc1ccc(-c2nnc(O)n2-c2cccc3ccc(F)cc23)c(O)c1] | 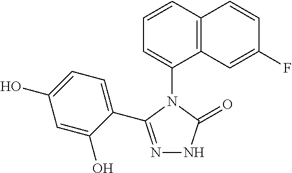 [SMILES: O=c1[nH]nc(-c2ccc(O)cc2O)n1-c1cccc2ccc(F)cc12] | 3-(2-Dihydroxy-phenyl)-4- (7-fluoro-naphthalen-1-yl)-5- hydroxy-[1,2,4] triazole |
| 146 | 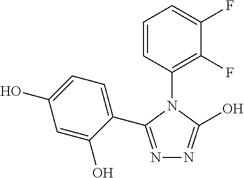 [SMILES: Oc1ccc(-c2nnc(O)n2-c2cccc(F)c2F)c(O)c1] | 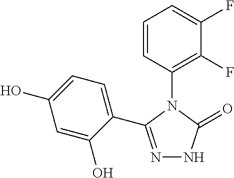 [SMILES: O=c1[nH]nc(-c2ccc(O)cc2O)n1-c1cccc(F)c1F] | 3-(2,4-Dihydroxy-phenyl)-4- (2,3-difluorophenyl)-5- hydroxy-[1,2,4] triazole |
| 147 | 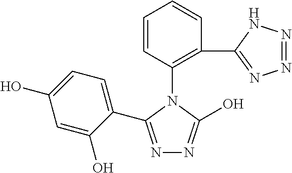 [SMILES: Oc1ccc(-c2nnc(O)n2-c2ccccc2-c2nnn[nH]2)c(O)c1] | 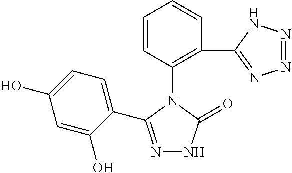 [SMILES: O=c1[nH]nc(-c2ccc(O)cc2O)n1-c1ccccc1-c1nnn[nH]1] | 3-(2,4-Dihydroxy-phenyl)-4- [2-(1H-tetrazol-5-yl)- phenyl]-5-hydroxy-[1,2,4] triazole |
| 148 | 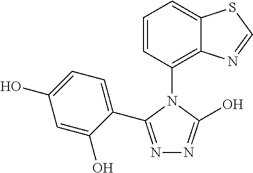 [SMILES: Oc1ccc(-c2nnc(O)n2-c2cccc3scnc23)c(O)c1] | 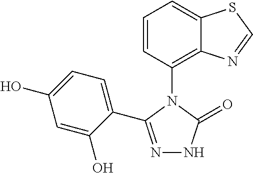 [SMILES: O=c1[nH]nc(-c2ccc(O)cc2O)n1-c1cccc2scnc12] | 3-(2,4-Dihydroxy-phenyl)-4- (benzothiazol-4-yl)-5- hydroxy-[1,2,4] triazole |
| 149 | 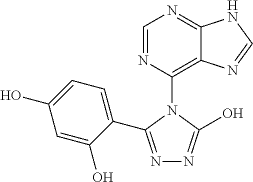 [SMILES: Oc1ccc(-c2nnc(O)n2-c2ncnc3[nH]cnc23)c(O)c1] | 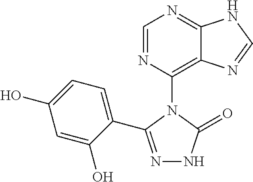 [SMILES: O=c1[nH]nc(-c2ccc(O)cc2O)n1-c1ncnc2[nH]cnc12] | 3-(2,4-Dihydroxy-phenyl)-4- (9H-purin-6-yl)-5-hydroxy- [1,2,4] triazole |
| 150 | 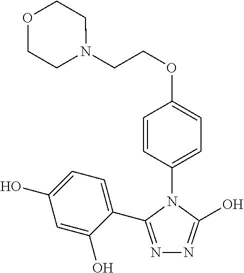 [SMILES: Oc1ccc(-c2nnc(O)n2-c2ccc(OCCN3CCOCC3)cc2)c(O)c1] | 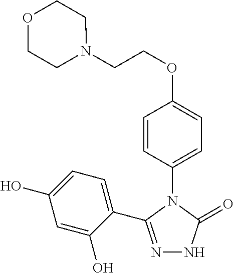 [SMILES: O=c1[nH]nc(-c2ccc(O)cc2O)n1-c1ccc(OCCN2CCOCC2)cc1] | 3-(2,4-Dihydroxy-phenyl)-4- {4-[2-(moropholin-1-yl)- ethoxy]-phenyl}-5-hydroxy- [1,2,4] triazole |
| 151 | 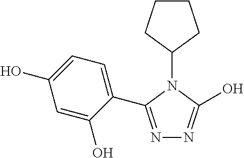 [SMILES: Oc1ccc(-c2nnc(O)n2C2CCCC2)c(O)c1] | 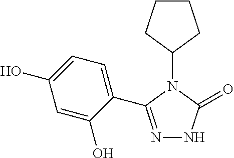 [SMILES: O=c1[nH]nc(-c2ccc(O)cc2O)n1C1CCCC1] | 3-(2,4-Dihydroxy-phenyl)-4- cyclopentyl-5-hydroxy- [1,2,4] triazole |
| 152 | 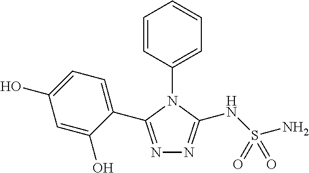 [SMILES: NS(=O)(=O)Nc1nnc(-c2ccc(O)cc2O)n1-c1ccccc1] | | 3-(2,4-Dihydroxy-phenyl)-4- phenyl-5-(sulfamoylamino)- [1,2,4] triazole |
| 153 | 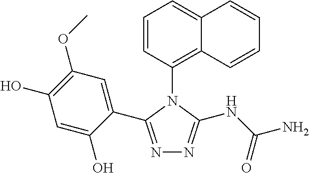 [SMILES: COc1cc(-c2nnc(NC(N)=O)n2-c2cccc3ccccc23)c(O)cc1O] | | 3-(2,4-Dihydroxy-5- methoxy-phenyl)-4- (naphthalene-1-yl)-5-ureido- [1,2,4] triazole |
| 154 | 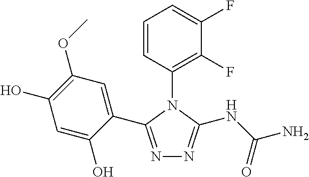 [SMILES: COc1cc(-c2nnc(NC(N)=O)n2-c2cccc(F)c2F)c(O)cc1O] | | 3-(2,4-Dihydroxy-5- methoxy-phenyl)-4-(2,3- difluorophenyl)-5-ureido- [1,2,4] triazole |
| 155 | 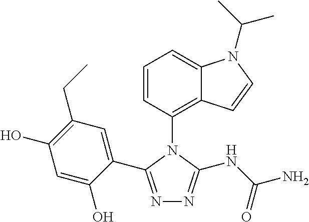 [SMILES: CCc1cc(-c2nnc(NC(N)=O)n2-c2cccc3c2ccn3C(C)C)c(O)cc1O] | | 3-(2,4-Dihydroxy-5-ethyl- phenyl)-4-(1-isopropyl-indol- 4-yl)-5-ureido-[1,2,4] triazole |
| 156 | 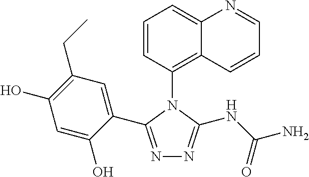 [SMILES: CCc1cc(-c2nnc(NC(N)=O)n2-c2cccc3ncccc23)c(O)cc1O] | | 3-(2,4-Dihydroxy-5-ethyl- phenyl)-4-(quinolin-5-yl)-5- ureido-[1,2,4] triazole |
| 157 | 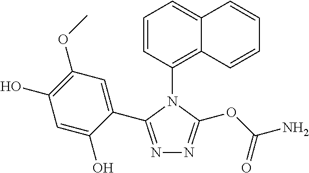 [SMILES: COc1cc(-c2nnc(OC(N)=O)n2-c2cccc3ccccc23)c(O)cc1O] | | 3-(2,4-Dihydroxy-5- methoxy-phenyl)-4- (naphthalene-1-yl)-5- carbamoyloxy-[1,2,4] triazole |
| 158 | 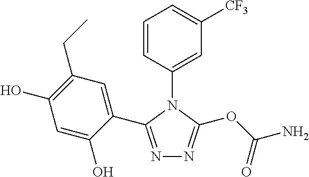 [SMILES: CCc1cc(-c2nnc(OC(N)=O)n2-c2cccc(C(F)(F)F)c2)c(O)cc1O] | | 3-(2,4-Dihydroxy-5-ethyl- phenyl)-4-(3-trifluoromethyl- phenyl)-5-carbamoyloxy- [1,2,4] triazole |
| 159 | 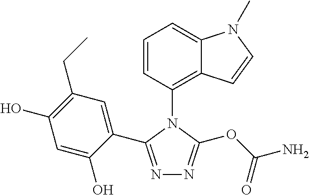 [SMILES: CCc1cc(-c2nnc(OC(N)=O)n2-c2cccc3c2ccn3C)c(O)cc1O] | | 3-(2,4-Dihydroxy-5-ethyl- phenyl)-4-(1-methyl-indol-4- yl)-5-carbamoyloxy-[1,2,4] triazole |
| 160 | 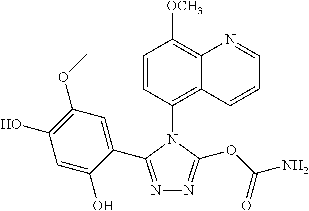 [SMILES: COc1cc(-c2nnc(OC(N)=O)n2-c2ccc(OC)c3ncccc23)c(O)cc1O] | | 3-(2,4-Dihydroxy-5- methoxy-phenyl)-4-(8- methoxy-quinolin-5-yl)-5- carbamoyloxy-[1,2,4] triazole |
| 161 | 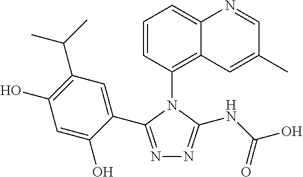 [SMILES: Cc1cnc2cccc(-n3c(NC(=O)O)nnc3-c3cc(C(C)C)c(O)cc3O)c2c1] | | 3-(2,4-Dihydroxy-5- isopropyl-phenyl)-4-(3- methyl-quinolin-5-yl)-5- carboxyamino-[1,2,4] triazole |
| 162 | 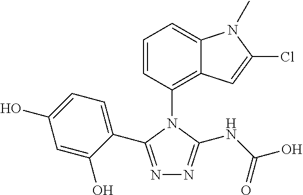 [SMILES: Cn1c(Cl)cc2c(-n3c(NC(=O)O)nnc3-c3ccc(O)cc3O)cccc21] | | 3-(2,4-Dihydroxy-phenyl)-4- (1-methyl-2-chloro-indol-4- yl)-5-carbamoyloxy-[1,2,4] triazole |
| 163 | 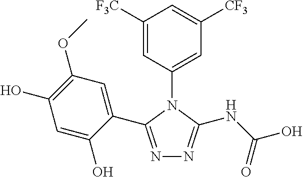 [SMILES: COc1cc(-c2nnc(NC(=O)O)n2-c2cc(C(F)(F)F)cc(C(F)(F)F)c2)c(O)cc1O] | | 3-(2,4-Dihydroxy-5- methoxy-phenyl)-4-[3,5-di- (trifluoromethyl)-phenyl]-5- carbamoyloxy-[1,2,4] triazole |
| 164 | 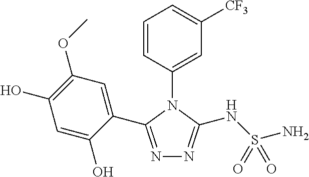 [SMILES: COc1cc(-c2nnc(NS(N)(=O)=O)n2-c2cccc(C(F)(F)F)c2)c(O)cc1O] | | 3-(2,4-Dihydroxy-5- methoxy-phenyl)-4-(3- trifluoroinethyl-phenyl)-5- (sulfamoylamino)-[1,2,4] triazole |
| 165 | 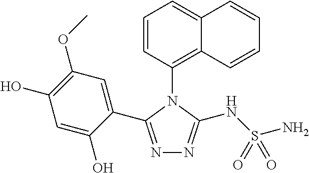 [SMILES: COc1cc(-c2nnc(NS(N)(=O)=O)n2-c2cccc3ccccc23)c(O)cc1O] | | 3-(2,4-Dihydroxy-5- methoxy-phenyl)-4- (naphthalene-1-yl)-5- (sulfamoylamino)-[1,2,4] triazole |
| 166 | 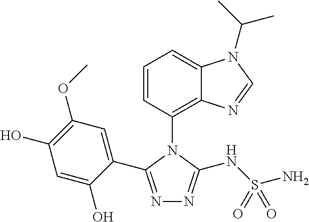 [SMILES: COc1cc(-c2nnc(NS(N)(=O)=O)n2-c2cccc3c2ncn3C(C)C)c(O)cc1O] | | 3-(2,4-Dihydroxy-5- methoxy-phenyl)-4-(1- isopropyl-benzoimidazol-4- yl)-5-(sulfamoylamino)- [1,2,4] triazole |
| 167 | 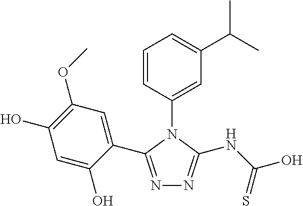 [SMILES: COc1cc(-c2nnc(NC(O)=S)n2-c2cccc(C(C)C)c2)c(O)cc1O] | | 3-(2,4-Dihydroxy-5- methoxy-phenyl)-4-(3- isopropylphenyl)-5- (thiocarboxyamino)-[1,2,4] triazole |
| 168 | 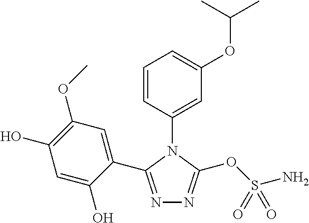 [SMILES: COc1cc(-c2nnc(OS(N)(=O)=O)n2-c2cccc(OC(C)C)c2)c(O)cc1O] | | 3-(2,4-Dihydroxy-5- methoxy-phenyl)-4-(3- isopropyloxy-phenyl)-5- (sulfamoyloxy)-[1,2,4] triazole |
| 169 | 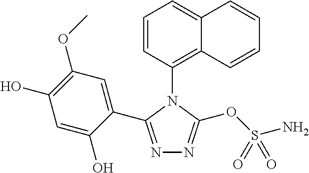 [SMILES: COc1cc(-c2nnc(OS(N)(=O)=O)n2-c2cccc3ccccc23)c(O)cc1O] | | 3-(2,4-Dihydroxy-5- methoxy-phenyl)-4- (naphthalene-1-yl)-5- (sulfamoyloxy)-[1,2,4] triazole |
| 170 | 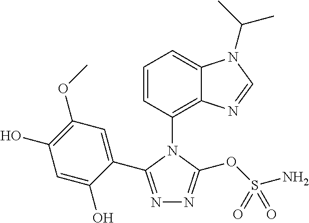 [SMILES: COc1cc(-c2nnc(OS(N)(=O)=O)n2-c2cccc3c2ncn3C(C)C)c(O)cc1O] | | 3-(2,4-Dihydroxy-5- methoxy-phenyl)-4-(1- isopropyl-benzoimidazol-4- yl)-5-(sulfamoyloxy)-[1,2,4] triazole |
| 171 | 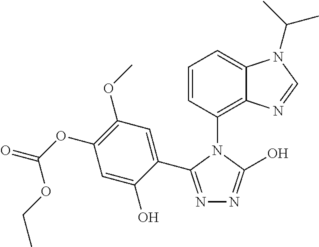 [SMILES: CCOC(=O)Oc1cc(O)c(-c2nnc(O)n2-c2cccc3c2ncn3C(C)C)cc1OC] | 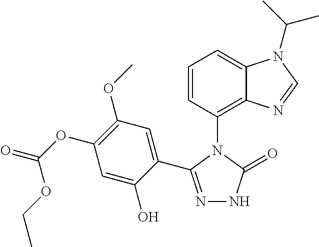 [SMILES: CCOC(=O)Oc1cc(O)c(-c2n[nH]c(=O)n2-c2cccc3c2ncn3C(C)C)cc1OC] | 3-(2-Hydroxy-4- ethoxycarbonyoxy-5- methoxy-phenyl)-4-(1- isopropyl-benzoimidazol-4- yl)-5-hydroxy-[1,2,4] triazole |
| 172 | 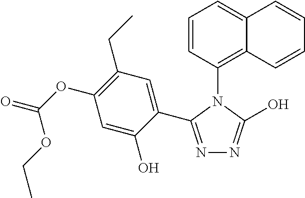 [SMILES: CCOC(=O)Oc1cc(O)c(-c2nnc(O)n2-c2cccc3ccccc23)cc1CC] | 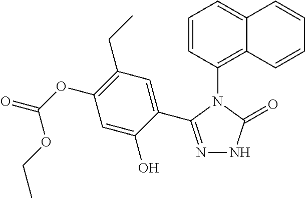 [SMILES: CCOC(=O)Oc1cc(O)c(-c2n[nH]c(=O)n2-c2cccc3ccccc23)cc1CC] | 3-(2-Hydroxy-4- ethoxycarbonyoxy-5-ethyl- phenyl)-4-(naphthalin-2-yl)- 5-hydroxy-[1,2,4] triazole |
| 173 | 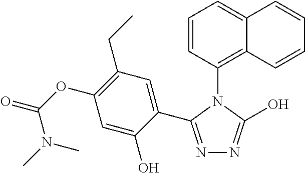 [SMILES: CCc1cc(-c2nnc(O)n2-c2cccc3ccccc23)c(O)cc1OC(=O)N(C)C] | 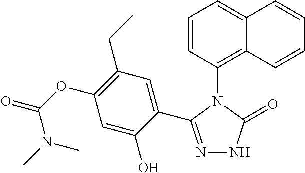 [SMILES: CCc1cc(-c2n[nH]c(=O)n2-c2cccc3ccccc23)c(O)cc1OC(=O)N(C)C] | 3-[2-Hydroxy-4-(dimethyl- carbamoyoxy)-5-ethyl- phenyl]-4-(naphthalin-2-yl)- 5-hydroxy-[1,2,4] triazole |
| 174 | 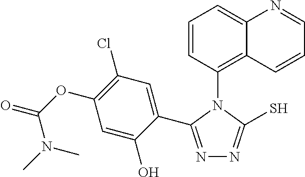 [SMILES: CN(C)C(=O)Oc1cc(O)c(-c2nnc(S)n2-c2cccc3ncccc23)cc1Cl] | 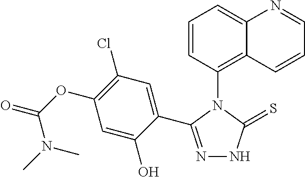 [SMILES: CN(C)C(=O)Oc1cc(O)c(-c2n[nH]c(=S)n2-c2cccc3ncccc23)cc1Cl] | 3-[2-Hydroxy-4-(dimethyl- carbamoyoxy)-5-chloro- phenyl]-4-(quinolin-5-yl)-5- mercapto-[1,2,4] triazole |
| 175 | 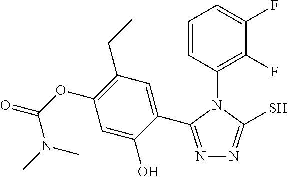 [SMILES: CCc1cc(-c2nnc(S)n2-c2cccc(F)c2F)c(O)cc1OC(=O)N(C)C] | 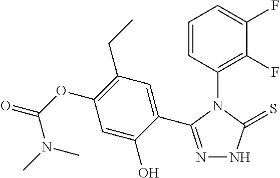 [SMILES: CCc1cc(-c2n[nH]c(=S)n2-c2cccc(F)c2F)c(O)cc1OC(=O)N(C)C] | 3-[2-Hydroxy-4-(dimethyl- carbamoyoxy)-5-ethyl- phenyl]-4-(2,3-difluoro- phenyl)-5-mercapto[1,2,4] triazole |
| 176 | 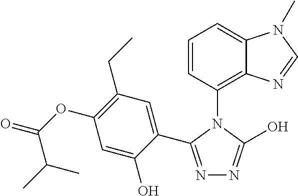 [SMILES: CCc1cc(-c2nnc(O)n2-c2cccc3c2ncn3C)c(O)cc1OC(=O)C(C)C] | 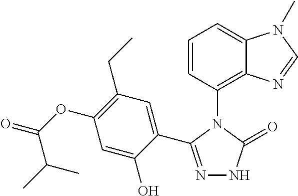 [SMILES: CCc1cc(-c2n[nH]c(=O)n2-c2cccc3c2ncn3C)c(O)cc1OC(=O)C(C)C] | 3-[2-Hydroxy-4- isobulyryloxy-5-ethyl- phenyl]-4-(1-methyl-benzo- imidazol-4-yl)-5-hydroxy- [1,2,4] triazole |
| 177 | 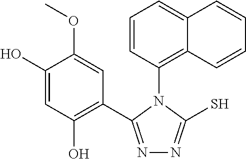 [SMILES: COc1cc(-c2nnc(S)n2-c2cccc3ccccc23)c(O)cc1O] | 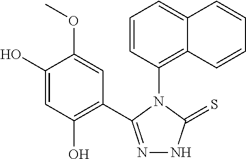 [SMILES: COc1cc(-c2n[nH]c(=S)n2-c2cccc3ccccc23)c(O)cc1O] | 3-(2,4-Dihydroxy-5- methoxy-phenyl)-4- (naphthalen-1-yl)-5- mercapto-[1,2,4] triazole |
| 178 | 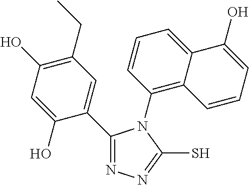 [SMILES: CCc1cc(-c2nnc(S)n2-c2cccc3c(O)cccc23)c(O)cc1O] | 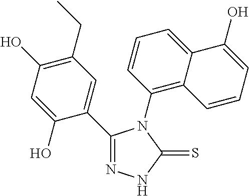 [SMILES: CCc1cc(-c2n[nH]c(=S)n2-c2cccc3c(O)cccc23)c(O)cc1O] | 3-(2,4-Dihydroxy-5-ethyl- phenyl)-4-(5-hydroxy- naphthalen-1-yl)-5-mercapto- [1,2,4] triazole |
| 179 | 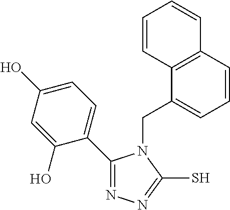 [SMILES: Oc1ccc(-c2nnc(S)n2Cc2cccc3ccccc23)c(O)c1] | 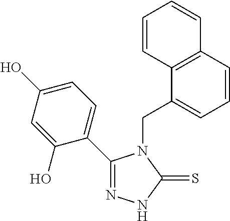 [SMILES: Oc1ccc(-c2n[nH]c(=S)n2Cc2cccc3ccccc23)c(O)c1] | 3-(2,4-Dihydroxy-phenyl)-4- (naphthalen-1-ylmethyl)-5- mercapto-[1,2,4] triazole |
| 180 | 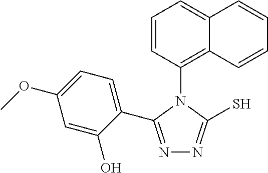 [SMILES: COc1ccc(-c2nnc(S)n2-c2cccc3ccccc23)c(O)c1] | 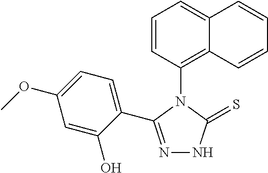 [SMILES: COc1ccc(-c2n[nH]c(=S)n2-c2cccc3ccccc23)c(O)c1] | 3-(2-Hydroxy-4- methoxyphenyl)-4- (naphthalen-1-yl)-5- mercapto-[1,2,4] triazole |
| 181 | 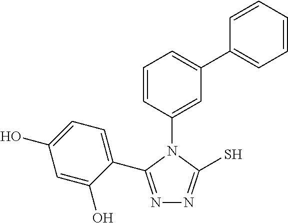 [SMILES: Oc1ccc(-c2nnc(S)n2-c2cccc(-c3ccccc3)c2)c(O)c1] | 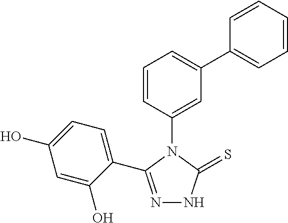 [SMILES: Oc1ccc(-c2n[nH]c(=S)n2-c2cccc(-c3ccccc3)c2)c(O)c1] | 3-(2,4-Dihydroxy-phenyl)-4- (biphenyl-3-yl)-5-mercapto- [1,2,4] triazole |
| 182 | 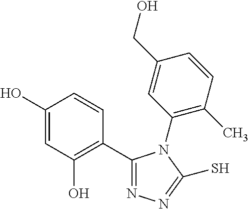 [SMILES: Cc1ccc(CO)cc1-n1c(S)nnc1-c1ccc(O)cc1O] | 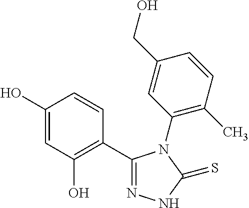 [SMILES: Cc1ccc(CO)cc1-n1c(-c2ccc(O)cc2O)n[nH]c1=S] | 3-(2,4-Dihydroxy-phenyl)-4- (2-methyl-5-hydroxymethyl- phenyl)-5-mercapto-[1,2,4] triazole |
| 183 | 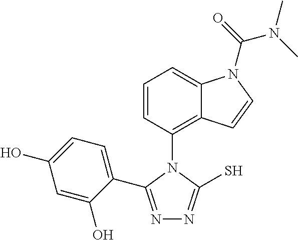 [SMILES: CN(C)C(=O)n1ccc2c(-n3c(S)nnc3-c3ccc(O)cc3O)cccc21] | 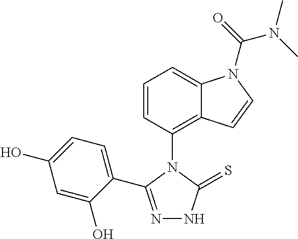 [SMILES: CN(C)C(=O)n1ccc2c(-n3c(-c4ccc(O)cc4O)n[nH]c3=S)cccc21] | 3-(2,4-Dihydroxy-phenyl)-4- (1-dimethylcarbamoyl-indol- 4-yl)-5-mercapto-[1,2,4] triazole |
| 184 | 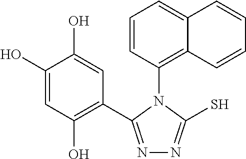 [SMILES: Oc1cc(O)c(-c2nnc(S)n2-c2cccc3ccccc23)cc1O] | 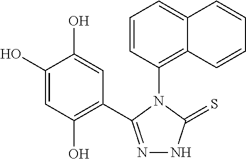 [SMILES: Oc1cc(O)c(-c2n[nH]c(=S)n2-c2cccc3ccccc23)cc1O] | 3-(2,4,5-Trihydroxy-phenyl)- 4-(naphthalene-1-yl)-5- mercapto-[1,2,4] triazole |
| 185 | 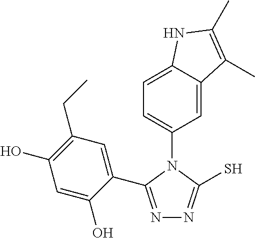 [SMILES: CCc1cc(-c2nnc(S)n2-c2ccc3[nH]c(C)c(C)c3c2)c(O)cc1O] | 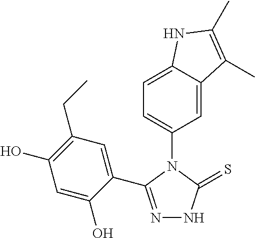 [SMILES: CCc1cc(-c2n[nH]c(=S)n2-c2ccc3[nH]c(C)c(C)c3c2)c(O)cc1O] | 3-(2,4-Dihydroxy-5-ethyl- phenyl)-4-(2,3-dimethyl- indol-5-yl)-5-mercapto- [1,2,4] triazole |
| 186 | 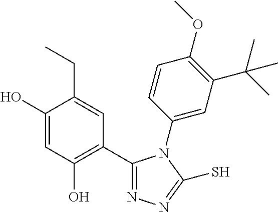 [SMILES: CCc1cc(-c2nnc(S)n2-c2ccc(OC)c(C(C)(C)C)c2)c(O)cc1O] | 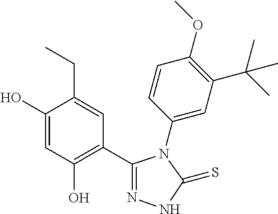 [SMILES: CCc1cc(-c2n[nH]c(=S)n2-c2ccc(OC)c(C(C)(C)C)c2)c(O)cc1O] | 3-(2,4-Dihydroxy-5-ethyl- phenyl)-4-(3-t-butyl-4- methoxy-phenyl)-5- mercapto-[1,2,4] triazole |
| 187 | 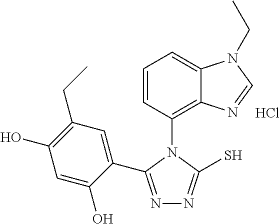 [SMILES: CCc1cc(-c2nnc(S)n2-c2cccc3c2ncn3CC)c(O)cc1O ; Cl] | 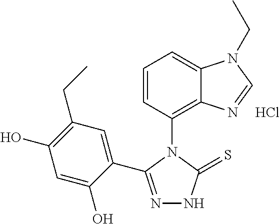 [SMILES: CCc1cc(-c2n[nH]c(=S)n2-c2cccc3c2ncn3CC)c(O)cc1O ; Cl] | 3-(2,4-Dihydroxy-5-ethyl- phenyl)-4-(1-ethyl-1H- benzoimidazol-4-yl)-5- mercapto-[1,2,4] triazole, HCl salt |
| 188 | 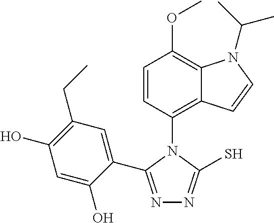 [SMILES: CCc1cc(-c2nnc(S)n2-c2ccc(OC)c3c2ccn3C(C)C)c(O)cc1O] | 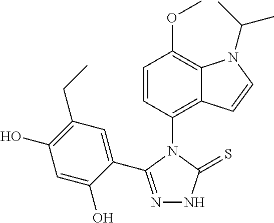 [SMILES: CCc1cc(-c2n[nH]c(=S)n2-c2ccc(OC)c3c2ccn3C(C)C)c(O)cc1O] | 3-(2,4-Dihydroxy-5-ethyl- phenyl)-4-(1-isopropyl-7- methoxy-indol-4-yl)-5- mercapto-[1,2,4] triazole |
| 189 | 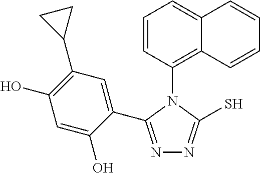 [SMILES: Oc1cc(O)c(C2CC2)cc1-c1nnc(S)n1-c1cccc2ccccc12] | 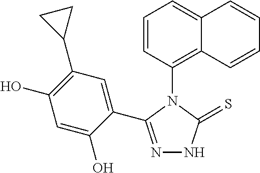 [SMILES: Oc1cc(O)c(C2CC2)cc1-c1n[nH]c(=S)n1-c1cccc2ccccc12] | 3-(2,4-Dihydroxy-5- cyclopropyl-phenyl)-4- (naphthalene-1-yl)-5- mercapto-[1,2,4] triazole |
| 190 | 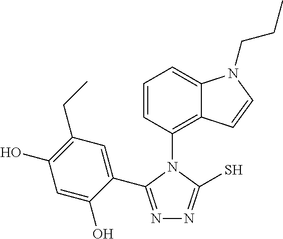 [SMILES: CCCn1ccc2c(-n3c(S)nnc3-c3cc(CC)c(O)cc3O)cccc21] | 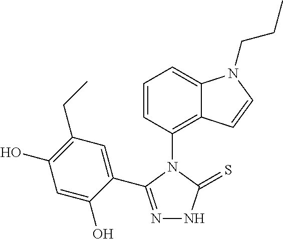 [SMILES: CCCn1ccc2c(-n3c(-c4cc(CC)c(O)cc4O)n[nH]c3=S)cccc21] | 3-(2,4-dihydroxy-5-ethyl- phenyl)-4-(1-propyl-indol-4- yl)-5-mercapto-[1,2,4] triazole |
| 191 | 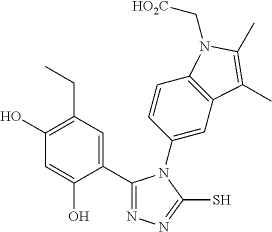 [SMILES: CCc1cc(-c2nnc(S)n2-c2ccc3c(c2)c(C)c(C)n3CC(=O)O)c(O)cc1O] | 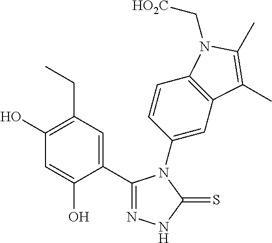 [SMILES: CCc1cc(-c2n[nH]c(=S)n2-c2ccc3c(c2)c(C)c(C)n3CC(=O)O)c(O)cc1O] | 3-(2,4-dihydroxy-5-ethyl- phenyl)-4-(1-acetyl-2,3- dimethyl-indol-5-yl)-5- mercapto-[1,2,4] triazole |
| 192 | 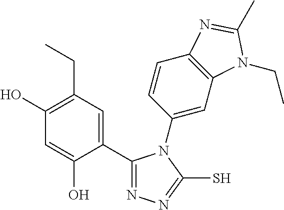 [SMILES: CCc1cc(-c2nnc(S)n2-c2ccc3nc(C)n(CC)c3c2)c(O)cc1O] | 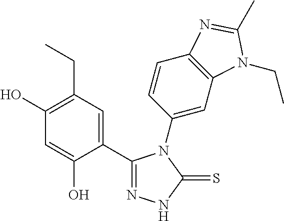 [SMILES: CCc1cc(-c2n[nH]c(=S)n2-c2ccc3nc(C)n(CC)c3c2)c(O)cc1O] | 3-(2,4-dihydroxy-5-ethyl- phenyl)-4-(2-methyl-3-ethyl- benzimidazol-5-yl)-5- mercapto-[1,2,4] triazole |
| 193 | 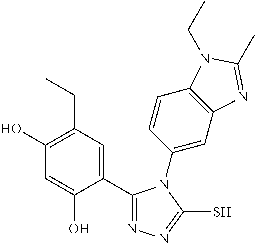 [SMILES: CCc1cc(-c2nnc(S)n2-c2ccc3c(c2)nc(C)n3CC)c(O)cc1O] | 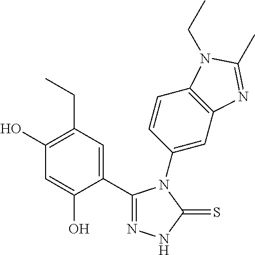 [SMILES: CCc1cc(-c2n[nH]c(=S)n2-c2ccc3c(c2)nc(C)n3CC)c(O)cc1O] | 3-(2,4-dihydroxy-5-ethyl- phenyl)-4-(1-ethyl-2-methyl- benzimidazol-5-yl)-5- mercapto-[1,2,4] triazole |
| 194 | 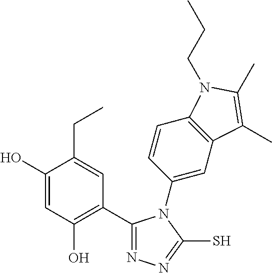 [SMILES: CCCn1c(C)c(C)c2cc(-n3c(S)nnc3-c3cc(CC)c(O)cc3O)ccc21] | 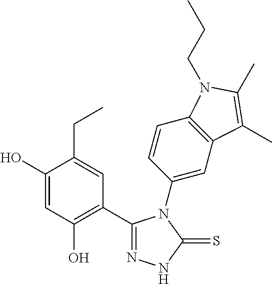 [SMILES: CCCn1c(C)c(C)c2cc(-n3c(-c4cc(CC)c(O)cc4O)n[nH]c3=S)ccc21] | 3-(2,4-dihydroxy-5-ethyl- phenyl)-4-(1-propyl-2,3- dimethyl-indol-5-yl)-5- mercapto-[1,2,4] triazole |
| 195 | 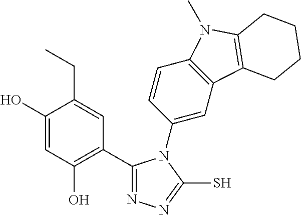 [SMILES: CCc1cc(-c2nnc(S)n2-c2ccc3c(c2)c2c(n3C)CCCC2)c(O)cc1O] | 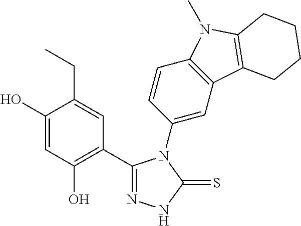 [SMILES: CCc1cc(-c2n[nH]c(=S)n2-c2ccc3c(c2)c2c(n3C)CCCC2)c(O)cc1O] | 3-(2,4-dihydroxy-5-ethyl- phenyl)-4-(N-methyl- tetrahydrocarbozol-7-yl)-5- mercapto-[1,2,4] triazole |
| 196 | 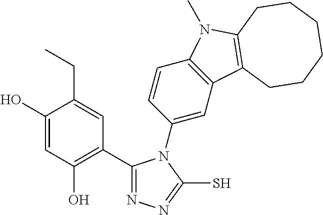 [SMILES: CCc1cc(-c2nnc(S)n2-c2ccc3c(c2)c2c(n3C)CCCCCC2)c(O)cc1O] | 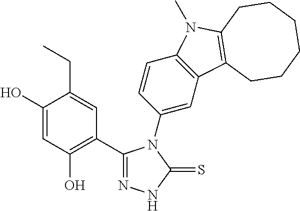 [SMILES: CCc1cc(-c2n[nH]c(=S)n2-c2ccc3c(c2)c2c(n3C)CCCCCC2)c(O)cc1O] | 3-(2,4-dihydroxy-5-ethyl- phenyl)-4-(N-methyl- cyclononan[a]indol-5-yl)-5- mercapto-[1,2,4] triazole |
| 197 | 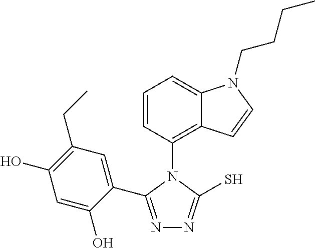 [SMILES: CCCCn1ccc2c(-n3c(S)nnc3-c3cc(CC)c(O)cc3O)cccc21] | 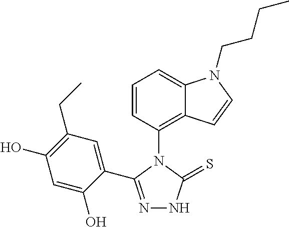 [SMILES: CCCCn1ccc2c(-n3c(-c4cc(CC)c(O)cc4O)n[nH]c3=S)cccc21] | 3-(2,4-dihydroxy-5-ethyl- phenyl)-4-(1-n-butyl-indol-4- yl)-5-mercapto-[1,2,4] triazole |
| 198 | 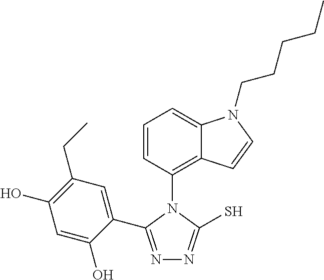 [SMILES: CCCCCn1ccc2c(-n3c(S)nnc3-c3cc(CC)c(O)cc3O)cccc21] | 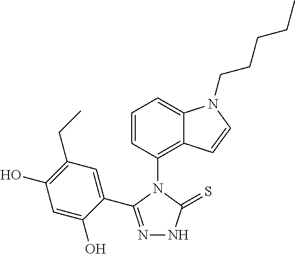 [SMILES: CCCCCn1ccc2c(-n3c(-c4cc(CC)c(O)cc4O)n[nH]c3=S)cccc21] | 3-(2,4-dihydroxy-5-ethyl- phenyl)-4-(1-n-pentyl-indol- 4-yl)-5-mercapto-[1,2,4] triazole |
| 199 | 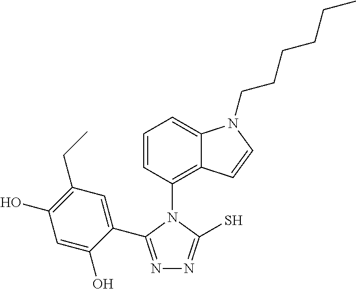 [SMILES: CCCCCCn1ccc2c(-n3c(S)nnc3-c3cc(CC)c(O)cc3O)cccc21] | 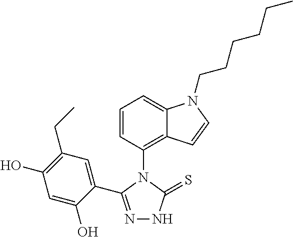 [SMILES: CCCCCCn1ccc2c(-n3c(-c4cc(CC)c(O)cc4O)n[nH]c3=S)cccc21] | 3-(2,4-dihydroxy-5-ethyl- phenyl)-4-(1-n-hexyl-indol-4- yl)-5-mercapto-[1,2,4] triazole |
| 200 | 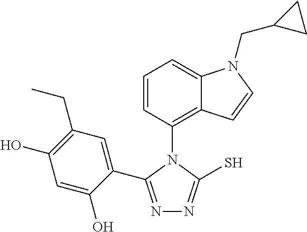 [SMILES: CCc1cc(-c2nnc(S)n2-c2cccc3c2ccn3CC2CC2)c(O)cc1O] | 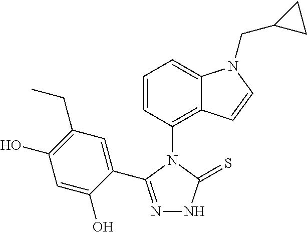 [SMILES: CCc1cc(-c2n[nH]c(=S)n2-c2cccc3c2ccn3CC2CC2)c(O)cc1O] | 3-(2,4-dihydroxy-5- cyclopropyl-phenyl)-4-(1-(1- methylcyclopropyl)-indol-4- yl)-5-mercapto-[1,2,4] triazole |
| 201 | 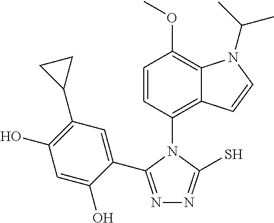 [SMILES: COc1ccc(-n2c(S)nnc2-c2cc(C3CC3)c(O)cc2O)c2ccn(C(C)C)c12] | 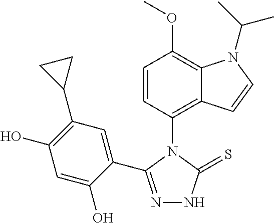 [SMILES: COc1ccc(-n2c(-c3cc(C4CC4)c(O)cc3O)n[nH]c2=S)c2ccn(C(C)C)c12] | 3-(2,4-dihydroxy-5- cyclopropyl-phenyl)-4-(1- isopropyl-7-methoxy-indol-4- yl)-5-mercapto-[1,2,4] triazole |
| 202 | 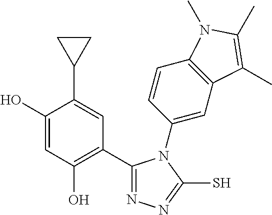 [SMILES: Cc1c(C)n(C)c2ccc(-n3c(S)nnc3-c3cc(C4CC4)c(O)cc3O)cc12] | 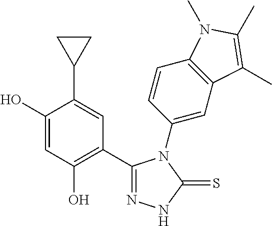 [SMILES: Cc1c(C)n(C)c2ccc(-n3c(-c4cc(C5CC5)c(O)cc4O)n[nH]c3=S)cc12] | 3-(2,4-dihydroxy-5- cyclopropyl-phenyl)-4-(1,2,3- trimethyl-indol-5-yl)-5- mercapto-[1,2,4] triazole |
| 203 | 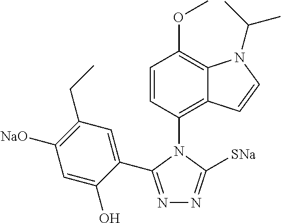 | | 3-(2,4-dihydroxy-5-ethyl- phenyl)-4-(1-isopropyl-7- methoxy-indol-4-yl)-5- mercapto-[1,2,4] triazole disodium salt |
| 204 | 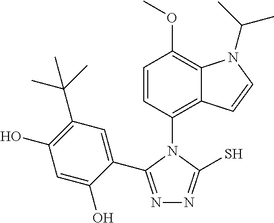 [SMILES: COc1ccc(-n2c(S)nnc2-c2cc(C(C)(C)C)c(O)cc2O)c2ccn(C(C)C)c12] | 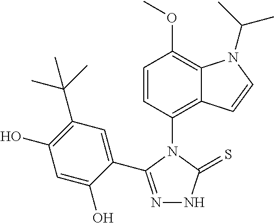 [SMILES: COc1ccc(-n2c(-c3cc(C(C)(C)C)c(O)cc3O)n[nH]c2=S)c2ccn(C(C)C)c12] | 3-(2,4-dihydroxy-5-tert- butyl-phenyl)-4-(1-isopropyl- 7-methoxy-indol-4-yl)-5- mercapto-[1,2,4] triazole |
| 205 | 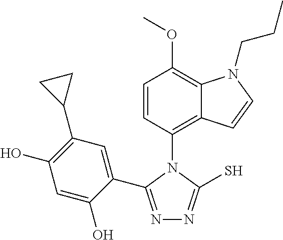 [SMILES: CCCn1ccc2c(-n3c(S)nnc3-c3cc(C4CC4)c(O)cc3O)ccc(OC)c21] | 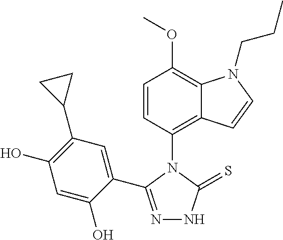 [SMILES: CCCn1ccc2c(-n3c(-c4cc(C5CC5)c(O)cc4O)n[nH]c3=S)ccc(OC)c21] | 3-(2,4-dihydroxy-5- cyclopropyl-phenyl)-4-(1- propyl-7-methoxy-indol-4- yl)-5-mercapto-[1,2,4] triazole |
| 206 | 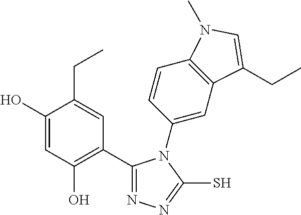 [SMILES: CCc1cc(-c2nnc(S)n2-c2ccc3c(c2)c(CC)cn3C)c(O)cc1O] | 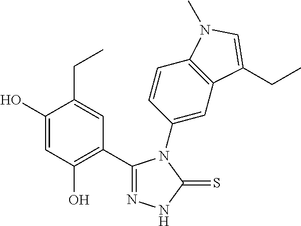 [SMILES: CCc1cc(-c2n[nH]c(=S)n2-c2ccc3c(c2)c(CC)cn3C)c(O)cc1O] | 3-(2,4-dihydroxy-5-ethyl- phenyl)-4-(1-methyl-3-ethyl- indol-5-yl)-5-mercapto- [1,2,4] triazole |
| 207 | 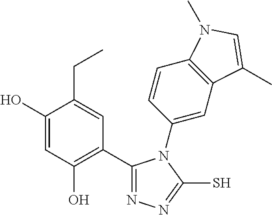 [SMILES: CCc1cc(-c2nnc(S)n2-c2ccc3c(c2)c(C)cn3C)c(O)cc1O] | 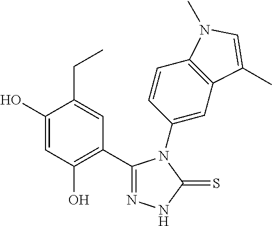 [SMILES: CCc1cc(-c2n[nH]c(=S)n2-c2ccc3c(c2)c(C)cn3C)c(O)cc1O] | 3-(2,4-dihydroxy-5-ethyl- phenyl)-4-(1,3-dimethyl- indol-5-yl)-5-mercapto- [1,2,4] triazole |
| 208 | 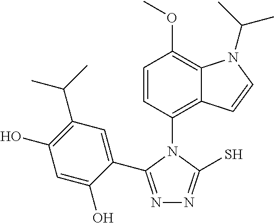 [SMILES: COc1ccc(-n2c(S)nnc2-c2cc(C(C)C)c(O)cc2O)c2ccn(C(C)C)c12] | 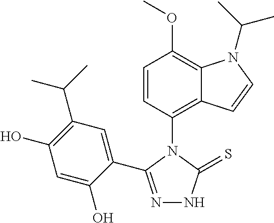 [SMILES: COc1ccc(-n2c(-c3cc(C(C)C)c(O)cc3O)n[nH]c2=S)c2ccn(C(C)C)c12] | 3-(2,4-dihydroxy-5- isopropyl-phenyl)-4-(1- isopropyl-7-methoxy-indol-4- yl)-5-mercapto-[1,2,4] triazole |
| 209 | 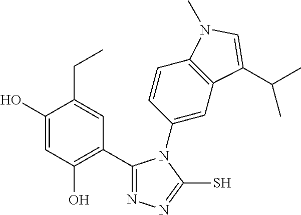 [SMILES: CCc1cc(-c2nnc(S)n2-c2ccc3c(c2)c(C(C)C)cn3C)c(O)cc1O] | 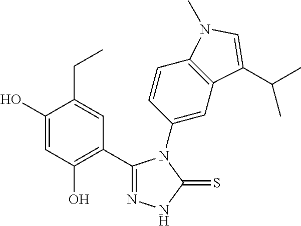 [SMILES: CCc1cc(-c2n[nH]c(=S)n2-c2ccc3c(c2)c(C(C)C)cn3C)c(O)cc1O] | 3-(2,4-dihydroxy-5-ethyl- phenyl)-4-(1-methyl-3- isopropyl-indol-5-yl)-5- mercapto-[1,2,4] triazole |
| 210 | 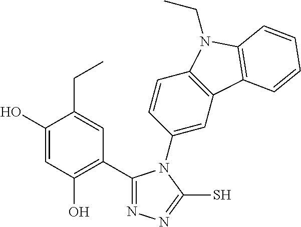 [SMILES: CCc1cc(-c2nnc(S)n2-c2ccc3c(c2)c2ccccc2n3CC)c(O)cc1O] | 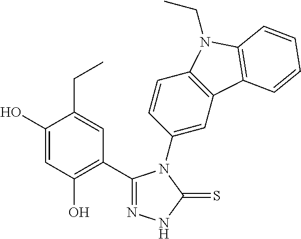 [SMILES: CCc1cc(-c2n[nH]c(=S)n2-c2ccc3c(c2)c2ccccc2n3CC)c(O)cc1O] | 3-(2,4-dihydroxy-5-ethyl- phenyl)-4-(N-ethyl-carbozol- 7-yl)-5-mercapto-[1,2,4] triazole |
| 211 | 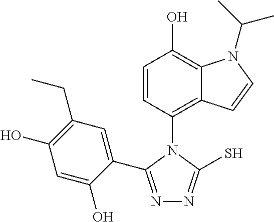 [SMILES: CCc1cc(-c2nnc(S)n2-c2ccc(O)c3c2ccn3C(C)C)c(O)cc1O] | 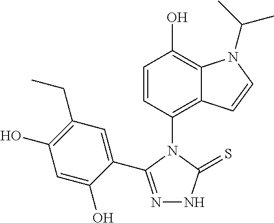 [SMILES: CCc1cc(-c2n[nH]c(=S)n2-c2ccc(O)c3c2ccn3C(C)C)c(O)cc1O] | 3-(2,4-dihydroxy-5-ethyl- phenyl)-4-(1-isopropyl-7- hydroxy-indol-4-yl)-5- mercapto-[1,2,4] triazole |
| 212 | 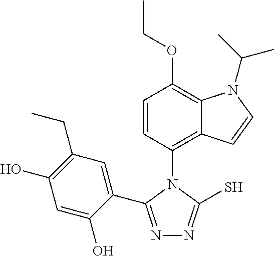 [SMILES: CCOc1ccc(-n2c(S)nnc2-c2cc(CC)c(O)cc2O)c2ccn(C(C)C)c12] | 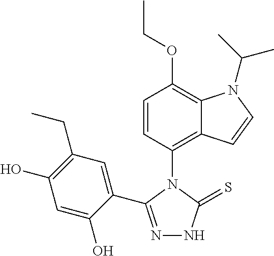 [SMILES: CCOc1ccc(-n2c(-c3cc(CC)c(O)cc3O)n[nH]c2=S)c2ccn(C(C)C)c12] | 3-(2,4-dihydroxy-5-ethyl- phenyl)-4-(1-isopropyl-7- ethoxy-indol-4-yl)-5- mercapto-[1,2,4] triazole |
| 213 | 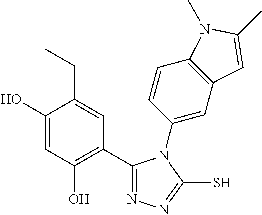 [SMILES: CCc1cc(-c2nnc(S)n2-c2ccc3c(c2)cc(C)n3C)c(O)cc1O] | 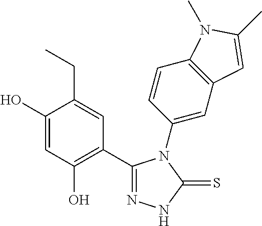 [SMILES: CCc1cc(-c2n[nH]c(=S)n2-c2ccc3c(c2)cc(C)n3C)c(O)cc1O] | 3-(2,4-dihydroxy-5-ethyl- phenyl)-4-(1,2-dimethyl- indol-5-yl)-5-mercapto- [1,2,4] triazole |
| 214 | 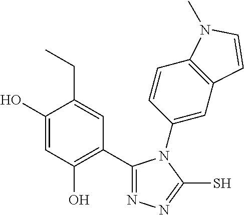 [SMILES: CCc1cc(-c2nnc(S)n2-c2ccc3c(ccn3C)c2)c(O)cc1O] | 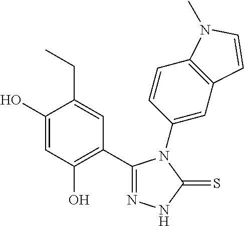 [SMILES: CCc1cc(-c2n[nH]c(=S)n2-c2ccc3c(ccn3C)c2)c(O)cc1O] | 3-(2,4-dihydroxy-5-ethyl- phenyl)-4-(N-methyl-indol-5- yl)-5-mercapto-[1,2,4] triazole |
| 215 | 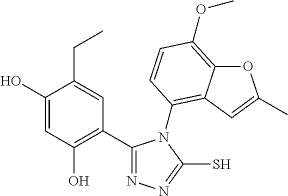 [SMILES: CCc1cc(-c2nnc(S)n2-c2ccc(OC)c3oc(C)cc23)c(O)cc1O] | 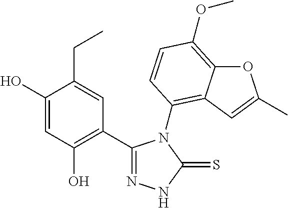 [SMILES: CCc1cc(-c2n[nH]c(=S)n2-c2ccc(OC)c3oc(C)cc23)c(O)cc1O] | 3-(2,4-dihydroxy-5-ethyl- phenyl)-4-(2-methyl-7- methoxy-benzofuran-4-yl)-5- mercapto-[1,2,4] triazole |
| 216 | 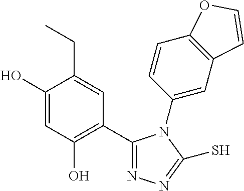 [SMILES: CCc1cc(-c2nnc(S)n2-c2ccc3occc3c2)c(O)cc1O] | 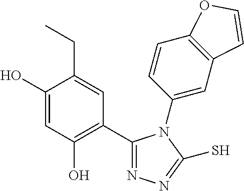 [SMILES: CCc1cc(-c2nnc(S)n2-c2ccc3occc3c2)c(O)cc1O] | 3-(2,4-dihydroxy-5-ethyl- phenyl)-4-(benzofuran-5-yl)- 5-mercapto-[1,2,4] triazole |
| 217 | 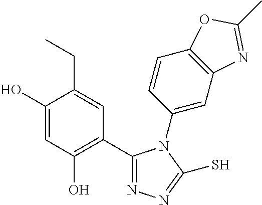 [SMILES: CCc1cc(-c2nnc(S)n2-c2ccc3oc(C)nc3c2)c(O)cc1O] | 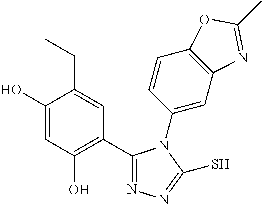 [SMILES: CCc1cc(-c2nnc(S)n2-c2ccc3oc(C)nc3c2)c(O)cc1O] | 3-(2,4-dihydroxy-5-ethyl- phenyl)-4-(2-methyl-1,3- benzoxaz-5-yl)-5-mercapto- [1,2,4] triazole |
| 218 | 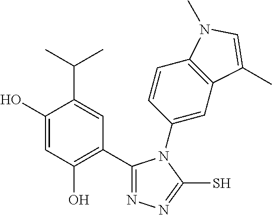 [SMILES: Cc1cn(C)c2ccc(-n3c(S)nnc3-c3cc(C(C)C)c(O)cc3O)cc12] | 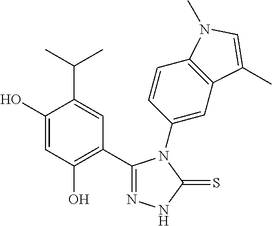 [SMILES: Cc1cn(C)c2ccc(-n3c(-c4cc(C(C)C)c(O)cc4O)n[nH]c3=S)cc12] | 3-(2,4-dihydroxy-5- isopropyl-phenyl)-4-(1,3- dimethyl-indol-5-yl)-5- mercapto-[1,2,4] triazole |
| 219 | 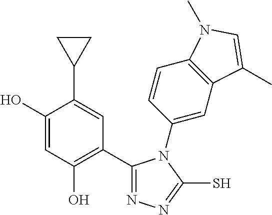 [SMILES: Cc1cn(C)c2ccc(-n3c(S)nnc3-c3cc(C4CC4)c(O)cc3O)cc12] | 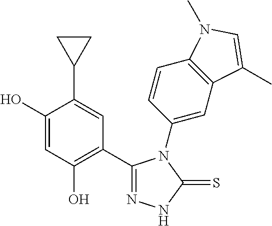 [SMILES: Cc1cn(C)c2ccc(-n3c(-c4cc(C5CC5)c(O)cc4O)n[nH]c3=S)cc12] | 3-(2,4-dihydroxy-5- cyclopropyl-phenyl)-4-(1,3- dimethyl-indol-5-yl)-5- mercapto-[1,2,4] triazole |
| 220 | 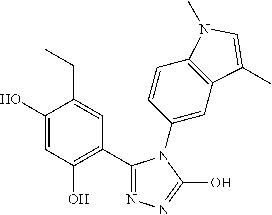 [SMILES: CCc1cc(-c2nnc(O)n2-c2ccc3c(c2)c(C)cn3C)c(O)cc1O] | 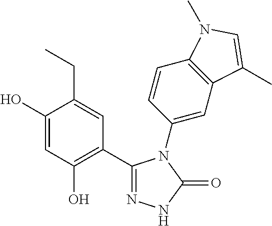 [SMILES: CCc1cc(-c2n[nH]c(=O)n2-c2ccc3c(c2)c(C)cn3C)c(O)cc1O] | 3-(2,4-dihydroxy-5-ethyl- phenyl)-4-(1,3-dimethyl- indol-5-yl)-5-hydroxy-[1,2,4] triazole |
| 221 | 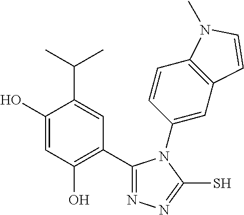 [SMILES: CC(C)c1cc(-c2nnc(S)n2-c2ccc3c(ccn3C)c2)c(O)cc1O] | 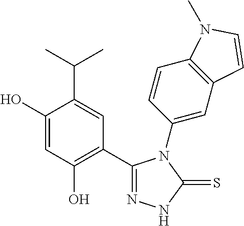 [SMILES: CC(C)c1cc(-c2n[nH]c(=S)n2-c2ccc3c(ccn3C)c2)c(O)cc1O] | 3-(2,4-dihydroxy-5- isopropyl-phenyl)-4-(N- methyl-indol-5-yl)-5- mercapto-[1,2,4] triazole |
| 222 | 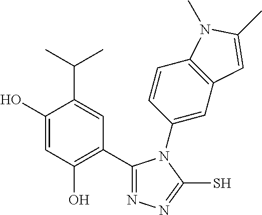 [SMILES: Cc1cc2cc(-n3c(S)nnc3-c3cc(C(C)C)c(O)cc3O)ccc2n1C] | 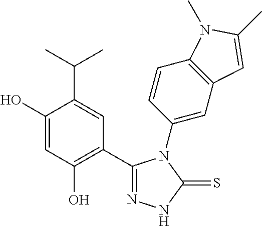 [SMILES: Cc1cc2cc(-n3c(-c4cc(C(C)C)c(O)cc4O)n[nH]c3=S)ccc2n1C] | 3-(2,4-dihydroxy-5- isopropyl-phenyl)-4-(1,2- dimethyl-indol-5-yl)-5- mercapto-[1,2,4] triazole |
| 223 | 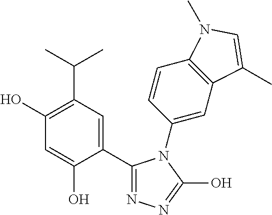 [SMILES: Cc1cn(C)c2ccc(-n3c(O)nnc3-c3cc(C(C)C)c(O)cc3O)cc12] | 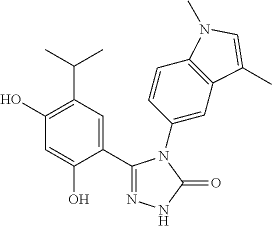 [SMILES: Cc1cn(C)c2ccc(-n3c(-c4cc(C(C)C)c(O)cc4O)n[nH]c3=O)cc12] | 3-(2,4-dihydroxy-5- isopropyl-phenyl)-4-(1,3- dimethyl-indol-5-yl)-5- hydroxy-[1,2,4] triazole |
| 224 | 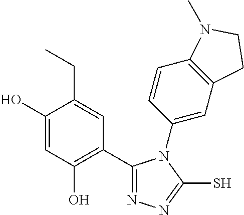 [SMILES: CCc1cc(-c2nnc(S)n2-c2ccc3c(c2)CCN3C)c(O)cc1O] | 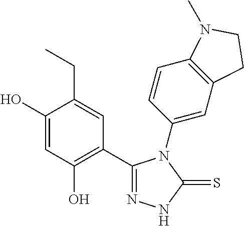 [SMILES: CCc1cc(-c2n[nH]c(=S)n2-c2ccc3c(c2)CCN3C)c(O)cc1O] | 3-(2,4-dihydroxy-5- cyclopropyl-phenyl)-4-(1- methyl-indol-5-yl)-5- mercapto-[1,2,4] triazole |
| 225 | 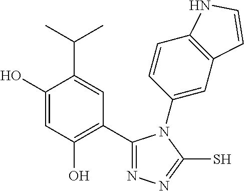 [SMILES: CC(C)c1cc(-c2nnc(S)n2-c2ccc3[nH]ccc3c2)c(O)cc1O] | 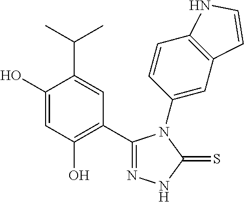 [SMILES: CC(C)c1cc(-c2n[nH]c(=S)n2-c2ccc3[nH]ccc3c2)c(O)cc1O] | 3-(2,4-dihydroxy-5- isopropyl-phenyl)-4-(1H- indol-5-yl)-5-mercapto- [1,2,4] triazole |
| 226 | 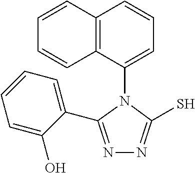 [SMILES: Oc1ccccc1-c1nnc(S)n1-c1cccc2ccccc12] | 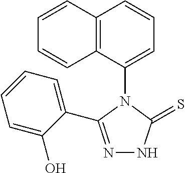 [SMILES: Oc1ccccc1-c1n[nH]c(=S)n1-c1cccc2ccccc12] | 3-(2-Hydroxyphenyl)-4- (naphthalen-1-yl)-5- mercapto-[1,2,4] triazole |
| 227 | 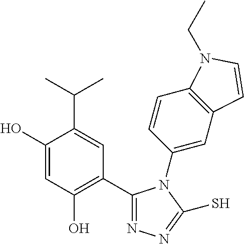 [SMILES: CCn1ccc2cc(-n3c(S)nnc3-c3cc(C(C)C)c(O)cc3O)ccc21] | 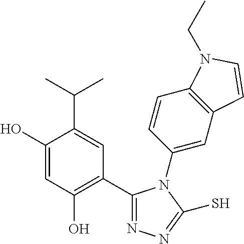 [SMILES: CCn1ccc2cc(-n3c(S)nnc3-c3cc(C(C)C)c(O)cc3O)ccc21] | 3-(2,4-dihydroxy-5- isopropyl-phenyl)-4-(1-ethyl- indol-5-yl)-5-mercapto- [1,2,4] triazole |
| 228 | 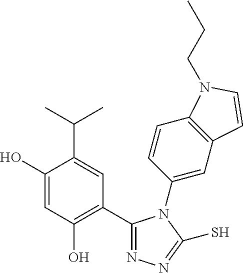 [SMILES: CCCn1ccc2cc(-n3c(S)nnc3-c3cc(C(C)C)c(O)cc3O)ccc21] | 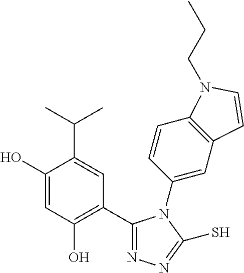 [SMILES: CCCn1ccc2cc(-n3c(S)nnc3-c3cc(C(C)C)c(O)cc3O)ccc21] | 3-(2,4-dihydroxy-5- isopropy1-phenyl)-4-(1- propyl-indol-5-yl)-5- mercapto-[1,2,4] triazole |
| 229 | 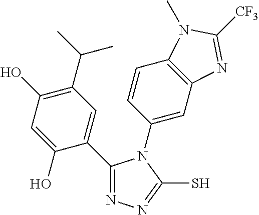 [SMILES: CC(C)c1cc(-c2nnc(S)n2-c2ccc3c(c2)nc(C(F)(F)F)n3C)c(O)cc1O] | 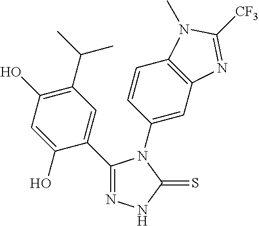 [SMILES: CC(C)c1cc(-c2n[nH]c(=S)n2-c2ccc3c(c2)nc(C(F)(F)F)n3C)c(O)cc1O] | 3-(2,4-dihydroxy-5- isopropyl-phenyl)-4-(1- methyl-2-trifluoromethyl- benzimidazol-5-yl)-5- mercapto-[1,2,4] triazole |
| 230 | 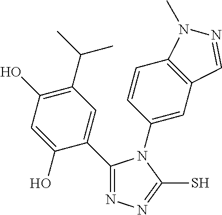 [SMILES: CC(C)c1cc(-c2nnc(S)n2-c2ccc3c(cnn3C)c2)c(O)cc1O] | 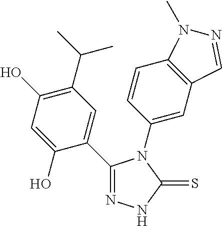 [SMILES: CC(C)c1cc(-c2n[nH]c(=S)n2-c2ccc3c(cnn3C)c2)c(O)cc1O] | 3-(2,4-dihydroxy-5- isopropyl-phenyl)-4-(1- methyl-indazol-5-yl)-5- mercapto-[1,2,4] triazole |
| 231 | 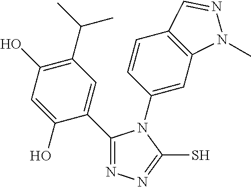 [SMILES: CC(C)c1cc(-c2nnc(S)n2-c2ccc3cnn(C)c3c2)c(O)cc1O] | 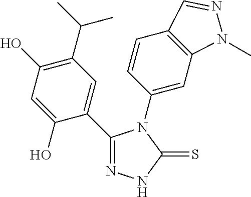 [SMILES: CC(C)c1cc(-c2n[nH]c(=S)n2-c2ccc3cnn(C)c3c2)c(O)cc1O] | 3-(2,4-dihydroxy-5- isopropyl-phenyl)-4-(1- methyl-indazol-6-yl)-5- mercapto-[1,2,4] triazole |
| 232 | 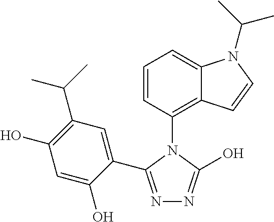 [SMILES: CC(C)c1cc(-c2nnc(O)n2-c2cccc3c2ccn3C(C)C)c(O)cc1O] | 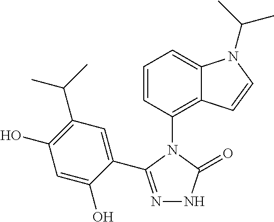 [SMILES: CC(C)c1cc(-c2n[nH]c(=O)n2-c2cccc3c2ccn3C(C)C)c(O)cc1O] | 3-(2,4-dihydroxy-5- isopropyl-phenyl)-4-(1- isopropyl-indol-4-yl)-5- hydroxy-[1,2,4] triazole |
| 233 | 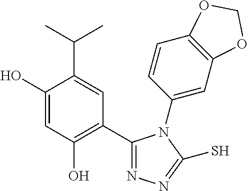 [SMILES: CC(C)c1cc(-c2nnc(S)n2-c2ccc3c(c2)OCO3)c(O)cc1O] | 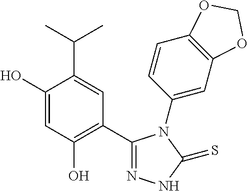 [SMILES: CC(C)c1cc(-c2n[nH]c(=S)n2-c2ccc3c(c2)OCO3)c(O)cc1O] | 3-(2,4-dihydroxy-5- isopropyl-phenyl)-4-(1,3- benzodiaxol-5-y1)-5- mercapto-[1,2,4] triazole |
| 234 | 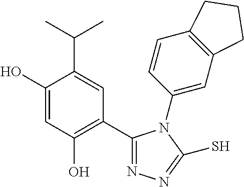 [SMILES: CC(C)c1cc(-c2nnc(S)n2-c2ccc3c(c2)CCC3)c(O)cc1O] | 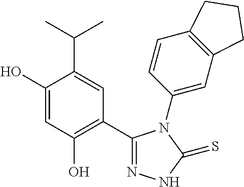 [SMILES: CC(C)c1cc(-c2n[nH]c(=S)n2-c2ccc3c(c2)CCC3)c(O)cc1O] | 3-(2,4-dihydroxy-5- isopropyl-phenyl)-4-(indan- 5-yl)-5-mercapto-[1,2,4] triazole |
| 235 | 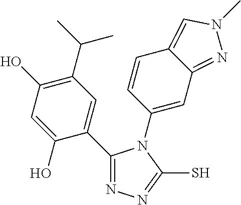 [SMILES: CC(C)c1cc(-c2nnc(S)n2-c2ccc3cn(C)nc3c2)c(O)cc1O] | 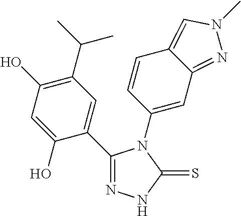 [SMILES: CC(C)c1cc(-c2n[nH]c(=S)n2-c2ccc3cn(C)nc3c2)c(O)cc1O] | 3-(2,4-dihydroxy-5- isopropyl-phenyl)-4-(2- methyl-indazol-6-yl)-5- mercapto-[1,2,4] triazole |
| 236 | 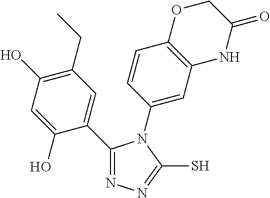 [SMILES: CCc1cc(-c2nnc(S)n2-c2ccc3c(c2)NC(=O)CO3)c(O)cc1O] | 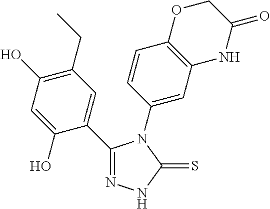 [SMILES: CCc1cc(-c2n[nH]c(=S)n2-c2ccc3c(c2)NC(=O)CO3)c(O)cc1O] | 3-(2,4-dihydroxy-5-ethyl- phenyl)-4-(3-oxo- benzo[1,4]oxazin-6-yl)-5- mercapto-[1,2,4] triazole |
| 237 | 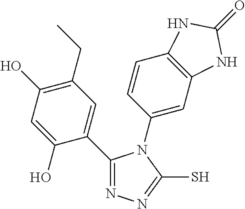 [SMILES: CCc1cc(-c2nnc(S)n2-c2ccc3[nH]c(=O)[nH]c3c2)c(O)cc1O] | 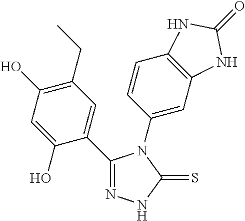 [SMILES: CCc1cc(-c2n[nH]c(=S)n2-c2ccc3[nH]c(=O)[nH]c3c2)c(O)cc1O] | 3-(2,4-dihydroxy-5-ethyl- phenyl)-4-(2-oxo-1,3- dihydro-benzoimidazol-5-yl)- 5-mercapto-[1,2,4] triazole |
| 238 | 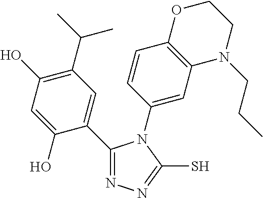 [SMILES: CCCN1CCOc2ccc(-n3c(S)nnc3-c3cc(C(C)C)c(O)cc3O)cc21] | 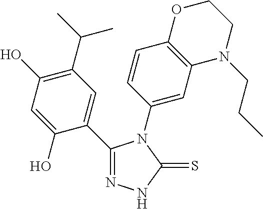 [SMILES: CCCN1CCOc2ccc(-n3c(-c4cc(C(C)C)c(O)cc4O)n[nH]c3=S)cc21] | 3-(2,4-dihydroxy-5- isopropyl-phenyl)-4-(2H- benzo[1,4]oxazin-6-yl)-5- mercapto-[1,2,4] triazole |
| 239 | 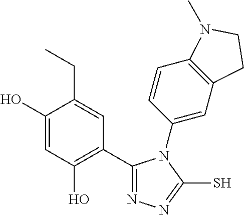 [SMILES: CCc1cc(-c2nnc(S)n2-c2ccc3c(c2)CCN3C)c(O)cc1O] | 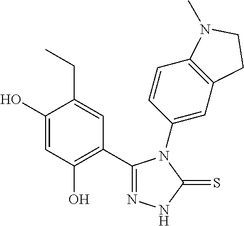 [SMILES: CCc1cc(-c2n[nH]c(=S)n2-c2ccc3c(c2)CCN3C)c(O)cc1O] | 4-Ethyl-6-[5-mercapto-4-(1- methyl-2,3-dihydro-1H- indol-5-yl)-4H-[1,2,4]triazol- 3-yl]-benzene-1,3-diol |
| 240 | 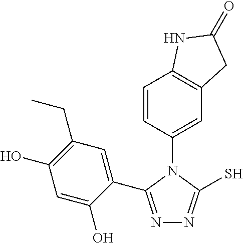 [SMILES: CCc1cc(-c2nnc(S)n2-c2ccc3c(c2)CC(=O)N3)c(O)cc1O] | 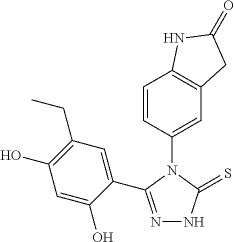 [SMILES: CCc1cc(-c2n[nH]c(=S)n2-c2ccc3c(c2)CC(=O)N3)c(O)cc1O] | 5-(3-(5-ethyl-2,4- dihydroxyphenyl)-5- mercapto-4H-1,2,4-triazol-4- yl)indolin-2-one |
| 241 | 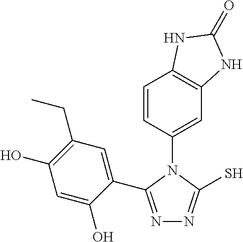 [SMILES: CCc1cc(-c2nnc(S)n2-c2ccc3[nH]c(=O)[nH]c3c2)c(O)cc1O] | 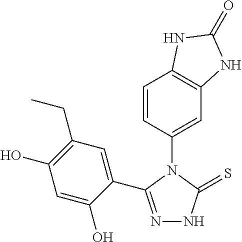 [SMILES: CCc1cc(-c2n[nH]c(=S)n2-c2ccc3[nH]c(=O)[nH]c3c2)c(O)cc1O] | 5-(3-(5-ethyl-2,4- dihydroxyphenyl)-5- mercapto-4H-1,2,4-triazol-4- yl)-1H-benzo[d]imidazol- 2(3H)-one |
| 242 | 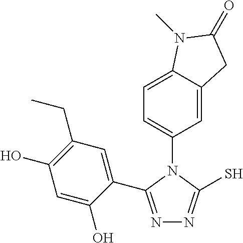 [SMILES: CCc1cc(-c2nnc(S)n2-c2ccc3c(c2)CC(=O)N3C)c(O)cc1O] | 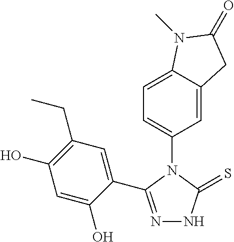 [SMILES: CCc1cc(-c2n[nH]c(=S)n2-c2ccc3c(c2)CC(=O)N3C)c(O)cc1O] | 5-(3-(5-ethyl-2,4- dihydroxyphenyl)-5- mercapto-4H-1,2,4-triazol-4- yl)-1-methylindolin-2-one |
| 243 | 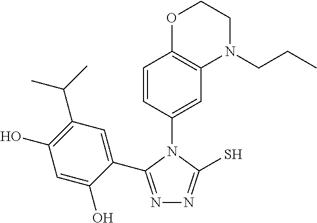 [SMILES: CCCN1CCOc2ccc(-n3c(S)nnc3-c3cc(C(C)C)c(O)cc3O)cc21] | 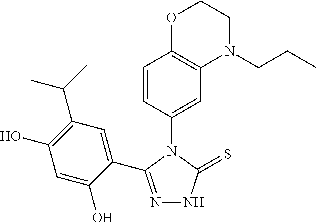 [SMILES: CCCN1CCOc2ccc(-n3c(-c4cc(C(C)C)c(O)cc4O)n[nH]c3=S)cc21] | 4-isopropyl-6-(5-mercapto-4- (4-propyl-3,4-dihydro-2H- benzo[b][1,4]oxazin-6-yl)- 4H-1,2,4-triazol-3- yl)benzene-1,3-diol |
| 244 | 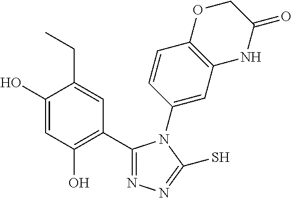 [SMILES: CCc1cc(-c2nnc(S)n2-c2ccc3c(c2)NC(=O)CO3)c(O)cc1O] | 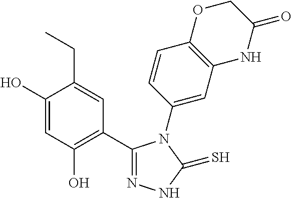 [SMILES: CCc1cc(-c2n[nH]c(=S)n2-c2ccc3c(c2)NC(=O)CO3)c(O)cc1O] | 6-(3-(5-ethyl-2,4- dihydroxyphenyl)-5- mercapto-4H-1,2,4-triazol-4- yl)-2H-benzo[b][1,4]oxazin- 3(4H)-one |
| 245 | 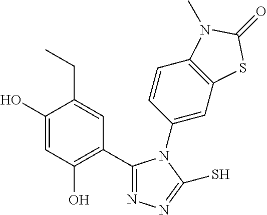 [SMILES: CCc1cc(-c2nnc(S)n2-c2ccc3c(c2)sc(=O)n3C)c(O)cc1O] | 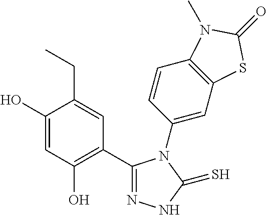 [SMILES: CCc1cc(-c2n[nH]c(=S)n2-c2ccc3c(c2)sc(=O)n3C)c(O)cc1O] | 6-(3-(5-ethyl-2,4- dihydroxyphenyl)-5- mercapto-4H-1,2,4-triazol-4- yl)-3-methylbenzo[d]thiazol- 2(3H)-one |
| 246 | 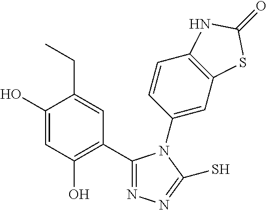 [SMILES: CCc1cc(-c2nnc(S)n2-c2ccc3[nH]c(=O)sc3c2)c(O)cc1O] | 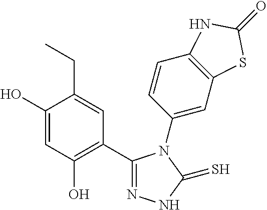 [SMILES: CCc1cc(-c2n[nH]c(=S)n2-c2ccc3[nH]c(=O)sc3c2)c(O)cc1O] | 6-(3-(5-ethyl-2,4- dihydroxyphenyl)-5- mercapto-4H-1,2,4-triazol-4- yl)benzo[d]thiazol-2(3H)-one |

The Hsp90 inhibitory compounds used in the pharmaceutical compositions and methods disclosed herein can be prepared according to the procedures disclosed in U.S. Patent Publication No. 2006/0167070, and WO2009/023211.

These triazolone compounds typically can form a tautomeric structure as shown below and as exemplified by the tautomeric structures shown in Table 1:

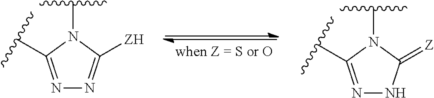

The pharmaceutical composition disclosed herein comprises a pharmaceutically acceptable organic solvent such as polyethylene glycol (PEG), dimethyl sulfoxide (DMSO), N-methylpyrolidinone (NMP), or glycerine, a pharmaceutically acceptable surfactant such as polysorbate 80, cremophor, or polyvinyl povidone (PVP), and a compound according to formulae (I)-(IV) or a compound in Table 1.

In one embodiment, the pharmaceutical composition comprises a pharmaceutically acceptable organic solvent, a pharmaceutically acceptable surfactant and a compound according to formulae (I)-(IV) or in Table 1, wherein the v/v ratio of organic solvent to surfactant is about 9:1. In one embodiment, the pharmaceutical composition comprises about 90% v/v PEG-300, about 10% v/v polysorbate 80, and compound 1. In one aspect of this embodiment, compound 1 is present in the pharmaceutical composition at a concentration of about 8 mg/mL.

In one embodiment, the pharmaceutical composition comprises a pharmaceutically acceptable organic solvent, pharmaceutically acceptable surfactant, a pharmaceutically acceptable co-solvent and a compound according to formulae (I)-(IV) or a compound in Table 1. In one embodiment, the pharmaceutical composition contains a v/v/v ratio of organic solvent to surfactant to co-solvent of about 39.35:35:25. In one embodiment, the pharmaceutical composition comprises about 39.35% v/v PEG-300, about 35% v/v polysorbate 80, about 25% v/v dehydrated alcohol, and compound 1. In one aspect of this embodiment, compound 1 is present in the pharmaceutical composition at a concentration of about 25 mg/mL.

In one embodiment, the pharmaceutical composition contains a v/v/v ratio of organic solvent to surfactant to co-solvent of about 37.5:37.5:25. In one embodiment, the pharmaceutical composition comprises about 37.5% v/v PEG-300, about 37.5% v/v polysorbate 80, about 25% v/v dehydrated alcohol, and compound 1. In one aspect of this embodiment, compound 1 is present in the pharmaceutical composition at a concentration of about 20 mg/mL.

In one embodiment, the pharmaceutical composition contains a v/v/v ratio of organic solvent to surfactant to co-solvent of about 55:25:20. In one embodiment, the pharmaceutical composition comprises about 55% v/v PEG-300, about 25% v/v polysorbate 80, about 20% v/v dehydrated alcohol, and compound 1. In one aspect of this embodiment, compound 1 is present in the pharmaceutical composition at a concentration of about 20 mg/mL.

In one embodiment, the invention includes a method of administering the pharmaceutical composition described herein to a subject in need thereof using a silicone catheter. In one embodiment, the silicone catheter is an in-dwelling catheter. In one embodiment, the pharmaceutical composition is administered to the subject via peripheral venous access. In one embodiment, the pharmaceutical composition is administered intravenously. In one embodiment, the invention also includes a kit for administering a pharmaceutical composition comprising a silicone catheter and one or more vials of the pharmaceutical composition described herein.

In one embodiment, the method includes treating a subject in need thereof comprising administering a pharmaceutical composition described herein at a dose of about 75 mg/m² to about 150 mg/m² of compound 1, approximately twice a week. In one embodiment, the dose of compound 1 is about 100 mg/m² to about 125 mg/m², administered twice a week. In one embodiment, the dose of compound 1 is about 120 mg/m², administered twice a week. In one embodiment, the dose of compound 1 is about 100 mg/m², administered twice a week. Other suitable dosing regimens are disclosed in U.S. Provisional Application Ser. No. 61/484,988, filed May 11, 2011, entitled DOSING REIMENS FOR TREATING CANCER WITH AN HSP90 INHIBITORY COMPOUND, the entire teachings of which are incorporated herein by reference.

In one embodiment, the method includes treating a subject in need thereof comprising administering a pharmaceutical composition described herein at a dose of about 120 mg/m² to about 250 mg/m² of compound 1, approximately once a week. In one embodiment, the dose of compound 1 is about 150 mg/m² to about 215 mg/m², administered once a week. In one embodiment, the dose of compound 1 is about 175 mg/m² to about 200 mg/m², administered once a week. In one embodiment, the dose of compound 1 is about 200 mg/m², administered once a week.

In one embodiment, the pharmaceutical composition comprises about 39.35% v/v PEG-300, about 35% v/v polysorbate 80, about 25% v/v dehydrated alcohol, and compound 1 at a concentration of about 25 mg/mL. In one aspect of this embodiment, the pharmaceutical composition is administered to a subject in need thereof once a week at a dose of about 200 mg/m². In one aspect of this embodiment, the pharmaceutical composition is administered to a subject in need thereof twice a week at a dose of about 120 mg/m². In any of these embodiments, the pharmaceutical composition is administered via a silicone in-dwelling catheter, or by peripheral venous access. In one embodiment, the pharmaceutical composition is administered to the subject via a silicone in-dwelling catheter.

In one embodiment, the pharmaceutical composition further comprises one or more other therapies (e.g., one or more therapeutic agents that are currently being used, have been used, are known to be useful or in development for use in the treatment or amelioration of a proliferative disorder, such as cancer, or one or more symptoms associated with said proliferative disorder). In one embodiment, the pharmaceutical composition further comprises an additional pharmaceutically acceptable co-solvent. In one embodiment, the pharmaceutical composition described herein is administered to a subject in addition to a second pharmaceutical composition containing one or more additional therapeutic agents.

In one embodiment, the two pharmaceutical compositions containing the two different therapies can be administered sequentially or concurrently. In one embodiment, the administration of a second pharmaceutical composition in addition to the pharmaceutical composition described herein can reduce the effective dosage of one or more of the therapies. In one embodiment, the two pharmaceutical compositions may be administered to a subject by the same or different routes of administration.

The pharmaceutical composition of the second therapeutic agent can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

### EXAMPLES

### Example 1

### Degradation of Hsp90 Client Proteins via Inhibition of Hsp90 Activity

Human high-Her2 breast carcinoma BT474 (HTB-20), SK-BR-3 (HTB-30) and MCF-7 breast carcinoma (HTB-22) from American Type Culture Collection, VA, USA were grown in Dulbecco's modified Eagle's medium with 4 mM L-glutamine and antibiotics (100 IU/ml penicillin and 100 μg/ml streptomycine; GibcoBRL). To obtain exponential cell growth, cells were trypsinized, counted and seeded at a cell density of 0.5×10⁶ cells /ml regularly, every 3 days. All experiments were performed on day 1 after cell passage.

After treatment with a compound described herein, cells were washed twice with 1× PBS/1% FBS, and then stained with anti-Her2-FITC (#340553, BD) for 30 min at 4° C. Cells were then washed three times in FACS buffer before the fixation in 0.5 ml 1% paraformaldehyde. Data was acquired on a FACS Calibur system. Isotype-matched controls were used to establish the non-specific staining of samples and to set the fluorescent markers. A total 10,000 events were recorded from each sample. Data were analyzed by using CellQuest software (BD Biosciences). The IC₅₀ ranges for Hsp90 inhibition by compounds described herein are listed below in Table 2.

TABLE 2
IC₅₀ Range of Compounds Described
Herein for Inhibition of Hsp90

| IC₅₀ Range | Compound Number |
|---|---|
| <3 μM | 1, 8, 13, 39, 49, 63, 76, 77, 79, 87, 88, 95, 96, 100, |
| | 103, 177, 178, 185, 188, 189, 195, 197, 198, 201, 202, |
| | 203, 204, 205, 206, 207, 208, 209, 211, 212, 213, 214, |
| | 215, 216, 218, 219, 220, 221, 222, 223 |
| 3 μM to μM | 2, 5, 6, 7, 9, 14, 27, 28, 34, 36, 38, 42, 48, 64, 70, 93, |
| | 97, 108, 122, 183, 184, 194, 196, 217 |
| 10 μM to 100 μM | 21, 22, 30, 51, 59, 60, 61, 62, 94, 98, 99, 102, 104, |
| | 123, 181, 182, 186, 187, 191, 192, 193, 199, 210 |

### Example 2

### Determination of the Amounts of Polysorbate 80 and PEG

Three formulations containing polysorbate 80 and PEG 300 were selected: 45% v/v polysorbate80-55%/v PEG 300, 50% v/v polysorbate80-50%/v PEG 300, and 55% v/v polysorbate80-45%/v PEG 300. Equilibrium solubility in each of these mixtures were measured by equilibrating excess solid in 2 mLs of the solution for 72 hours, and assaying the supernatant clear liquid using HPLC analysis. The results were summarized in Table 3.

TABLE 3
Solubility of Compound 1 in Different Ratios
of Polysorbate 80 and PEG 300 Solutions

| | Solubility |
|---|---|
| Sample | mg/mL |
| 50% PEG 300, 50% polysorbate 80 | 96.16 |
| 45% PEG 300, 55% polysorbate 80 | 84.69 |
| 40% PEG 300, 60% polysorbate 80 | 76.11 |

It can be observed from the solubility data that as the content of PEG 300 was reduced in the solution, the solubility of compound 1 decreased. In all three solutions solubilities of >75 mg/mL were achieved and hence would be sufficient for the formulation of compound 1 that as targeted <50 mg/mL which in turn upon dilution would be able to achieve desired concentrations in the dosing solutions.

### Example 3

### Formulation A

A stable formulation of compound 1 was created using 90% v/v PEG 300 and 10% v/v polysorbate 80 at a concentration of 8 mg/mL. This formulation was the drug product developed for the Phase 1 and IIa clinical studies. The drug product is packaged in a Type I glass amber vial, stoppered with a Flurotec® coated stopper, and sealed. Each vial has a deliverable volume of 12.5 mLs (equivalent to 100 mg/vial). The drug product is further diluted with 5% Dextrose for Injection (D5W) in infusion container (DEHP-free 500 mL) to a concentration range of 0.02 to 1.2 mg/mL and administered via infusion tubing (DEHP-free) with a 0.22μ end filter over an hour to the patient. The dosing solution once prepared must be administered within 3 hours.

Although Formulation A has been used to support Phase I and IIa clinical trials of compound 1, a more optimized and better solution was sought.

### Example 4

### Addition of Cosolvent(s)

Two cosolvents: propylene glycol and dehydrated alcohol were chosen in an effort to achieve a miscible solution with the polysorbate 80 and PEG 300 as well as lowering the viscosity of the drug product. It was reasoned that both propylene glycol and dehydrated alcohol were less viscous than PEG-300 or polysorbate 80, which would aid in lowering the viscosity of the current drug product. Dehydrated alcohol especially with its relatively lower viscosity would be the better choice among the two.

It was observed that propylene glycol did not help in aiding miscibility of PEG 300 and polysorbate 80 in the presence or absence of dehydrated alcohol. On the other hand dehydrated alcohol allowed several options to attain a miscible combination with polysorbate 80 and PEG 300. Addition of dehydrated alcohol not only addressed the miscibility but had an added advantage of lowering the viscosity. For instance the combination of 25% v/v dehydrated alcohol, 35% v/v polysorbate 80, and 40% v/v PEG 400 resulted in a clear, miscible solution with a kinematic viscosity of 48 cstoke, which was half of that of Formulation A—a significant improvement.

### Example 5

### Formulation Prototypes

Five Formulation Prototypes containing various combinations of dehydrated alcohol, polysorbate 80, and PEG 300 were designed to deliver 50 mg/m²-250 mg/m² compound 1 dose. The infusion solutions were physically and chemically stable for at least 10 hours. Physical and chemical stability of the solutions were collected up to 1 year and no significant changes in the assays were observed.

TABLE 4
Different Formulation Prototypes

| | | Dehydrated | Polysorbate | |
|---|---|---|---|---|
| Prototype | Compound 1 | Alcohol | 80 | PEG 300 |
| Prototype B1 | 35 mg/mL | 0% v/v | 50% v/v | 50% v/v |
| Prototype B2 | 25 mg/mL | 25% v/v | 35% v/v | 39.35% v/v |
| Prototype B3 | 20 mg/mL | 25% v/v | 37.5% v/v | 37.5% v/v |
| Prototype B4 | 20 mg/mL | 20% v/v | 25% v/v | 55% v/v |
| Prototype B5 | 10 mg/mL | 0% v/v | 15% v/v | 85% v/v |

### Example 6

### Optimized Formulation

The prototype formulation containing 25 mg/mL compound 1 in 25% v/v dehydrated alcohol, 35% v/v polysorbate 80, and 39.35% PEG 300 was surprisingly found to address all the limitations of the Phase 1 and IIa drug product. The advantages of the optimized formation include complete miscibility of the drug product solution, lowered viscosity by half, and increased infusion solution physical stability up to 8 hours. Moreover, the optimized formulation could be stored in a single vial containing 200 mg/m² dose. The formulation had excellent physical and chemical stability at 12 months, as summarized in Tables 5-13 below.

TABLE 5
Stability Results for Prototype B1containing 35 mg/mL
Compound 1 in 50% v/v Polysorbate 80, and 50%
PEG 300 Drug Product Stored at 40° C./75% RH
40° C./75% RH

| | Time (Months) | | | |
|---|---|---|---|---|
| | 0 | 0.5 | 1 | 3 |
| Appearance | Conforms | Conforms | Conforms | Conforms |

Potency

| | | | | |
|---|---|---|---|---|
| % LC | 99.4 | 101.1 | 100.9 | 101.7 |
| Concentration | 34.8 | 35.4 | 35.31 | 35.6 |
| (mg/mL) | | | | |

Related substances (% w/w)

| | | | | |
|---|---|---|---|---|
| Impurity A | <0.05 | <0.05 | <0.05 | <0.05 |
| Impurity B | <0.05 | <0.05 | <0.05 | <0.05 |
| Impurity C | 0.72 | 0.68 | 0.57 | 0.38 |
| Impurity D | <0.05 | <0.05 | <0.05 | <0.05 |
| Impurity E | <0.05 | <0.05 | <0.05 | <0.05 |
| Impurity F | 0.09 | 0.08 | 0.08 | 0.10 |
| Unknown at | 0.05 | 0.05 | 0.05 | 0.05 |
| RRT 0.22 | | | | |
| Total | 0.86 | 0.81 | 0.71 | 0.53 |
| Impurities | | | | |

Conforms = Clear, Colorless to yellow solution essentially free of visible particles.

TABLE 6
Stability Results for Prototype B2 containing 25 mg/mL
Compound 1 in 25% v/v Dehydrated Alcohol, 35% v/v Polysorbate 80,
and 39.35% PEG 300 Drug Product Stored at 40° C./75% RH
Test ID

| | Time (Months) | | | |
|---|---|---|---|---|
| | 0 | 1 | 3 | 6 |
| Appearance | Conforms | Conforms | Conforms | Conforms |
| Assay (% LC) | 101.6 | 101.2 | 99.6 | 98.8 |

Related substances (% w/w)

| | | | | |
|---|---|---|---|---|
| Impurity A | <0.10 | <0.10 | <0.10 | <0.10 |
| Impurity B | <0.10 | <0.10 | <0.10 | <0.10 |
| Impurity C | 0.72 (0.23) | 0.55 (0.17) | 0.32 (0.10) | 0.19 (<0.10) |
| Impurity D | <0.10 | <0.10 | <0.10 | <0.10 |
| Impurity E | <0.10 | <0.10 | <0.10 | <0.10 |
| Impurity F | <0.10 | <0.10 | <0.10 | <0.10 |
| Individual | <0.10 | <0.10 | <0.10 | <0.10 |
| Unknown | | | | |
| Total | 0.72 | 0.55 | 0.32 | 0.19 |
| Impurities | | | | |

Conforms = Clear colorless to pale yellow solution, essentially free of visible particles

TABLE 7
Stability Results for PrototypeB2 containing 25 mg/mL
Compound 1 in 25% v/v Dehydrated Alcohol, 35% v/v Polysorbate 80,
and 39.35% PEG 300 Drug Product Stored at 25° C./60% RH
Test ID

| | Time (Months) | | | |
|---|---|---|---|---|
| | 0 | 6 | 9 | 12 |
| Appearance | Conforms | Conforms | Conforms | Conforms |
| Assay (% LC) | 101.6 | 99.6 | 102.4 | 102.0 |

Related substances (% w/w)

| | | | | |
|---|---|---|---|---|
| Impurity A | <0.10 | <0.10 | <0.10 | <0.10 |
| Impurity B | <0.10 | <0.10 | <0.10 | <0.10 |
| Impurity C | 0.72 (0.23) | 0.51 (0.16) | 0.42 | 0.35 |
| Impurity D | <0.10 | <0.10 | <0.10 | <0.10 |
| Impurity E | <0.10 | <0.10 | <0.10 | <0.10 |
| Impurity F | <0.10 | <0.10 | 0.10 | 0.10 |
| Individual | <0.10 | <0.10 | <0.10 | <0.10 |
| Unknown | | | | |
| Total | 0.72 | 0.51 | 0.52 | 0.45 |
| Impurities | | | | |

Conforms = Clear colorless to pale yellow solution, essentially free of visible particles

TABLE 8
Stability Results for Prototype B2 containing 25 mg/mL
Compound 1 in 25% v/v Dehydrated Alcohol, 35% v/v Polysorbate 80,
and 39.35% PEG 300 Drug Product Stored at 2-8° C.
Test ID

| | Time (Months) | | | |
|---|---|---|---|---|
| | 0 | 6 | 9 | 12 |
| Appearance | Conforms | Conforms | Conforms | Conforms |
| Assay (% LC) | 101.6 | 100.0 | 102.0 | 99.2 |

Related substances (% w/w)

| | | | | |
|---|---|---|---|---|
| Impurity A | <0.10 | <0.10 | <0.10 | <0.10 |
| Impurity B | <0.10 | <0.10 | <0.10 | <0.10 |
| Impurity C | 0.72 (0.23) | 0.70 (0.33) | 0.73 | 0.72 |
| Impurity D | <0.10 | <0.10 | <0.10 | <0.10 |
| Impurity E | <0.10 | <0.10 | <0.10 | <0.10 |
| Impurity F | <0.10 | <0.10 | 0.10 | 0.10 |
| Individual | <0.10 | <0.10 | <0.10 | <0.10 |
| Unknown | | | | |
| Total | 0.72 | 0.70 | 0.83 | 0.82 |
| Impurities | | | | |

Conforms = Clear colorless to pale yellow solution, essentially free of visible particles

TABLE 9
Stability Results for Prototype B3 containing 20 mg/mL
Compound 1 in 25% v/v Dehydrated Alcohol, 37.5% v/v Polysorbate 80,
and 37.5% PEG 300 Drug Product Stored at 40° C./75% RH
Test ID

| | Time (Months) | | | |
|---|---|---|---|---|
| | 0 | 1 | 3 | 6 |
| Appearance | Conforms | Conforms | Conforms | Conforms |
| Assay (% LC) | 101.0 | 101.0 | 99.5 | 98.5 |

Related substances (% w/w)

| | | | | |
|---|---|---|---|---|
| Impurity A | <0.10 | <0.10 | <0.10 | <0.10 |
| Impurity B | <0.10 | <0.10 | <0.10 | <0.10 |
| Impurity C | 0.71 (0.23) | 0.34 (0.10) | 0.13 (<0.10) | <0.10 |
| Impurity D | <0.10 | <0.10 | <0.10 | <0.10 |
| Impurity E | <0.10 | <0.10 | <0.10 | <0.10 |
| Impurity F | <0.10 | <0.10 | <0.10 | <0.10 |
| Individual | <0.10 | <0.10 | <0.10 | <0.10 |
| Unknown | | | | |
| Total | 0.71 | 0.34 | 0.13 | <0.10 |
| Impurities | | | | |

Conforms = Clear colorless to pale yellow solution, essentially free of visible particles

TABLE 10
Stability Results for Prototype B3 containing 20 mg/mL
Compound 1 in 25% v/v Dehydrated Alcohol, 37.5% v/v Polysorbate 80,
and 37.5% PEG 300 Drug Product Stored at 25° C./60% RH
Test ID

| | Time (Months) | | | |
|---|---|---|---|---|
| | 0 | 6 | 9 | 12 |
| Appearance | Conforms | Conforms | Conforms | Conforms |
| Assay (% LC) | 101.0 | 99.5 | 102.5 | 100.5 |

Related substances (% w/w)

| | | | | |
|---|---|---|---|---|
| Impurity A | <0.10 | <0.10 | <0.10 | <0.10 |
| Impurity B | <0.10 | <0.10 | <0.10 | <0.10 |
| Impurity C | 0.71 (0.23) | 0.26 (<0.10) | 0.13 (<0.10) | 0.10 (<0.10) |
| Impurity D | <0.10 | <0.10 | <0.10 | <0.10 |
| Impurity E | <0.10 | <0.10 | <0.10 | <0.10 |
| Impurity F | <0.10 | <0.10 | 0.10 | 0.11 |
| Individual | <0.10 | <0.10 | <0.10 | <0.10 |
| Unknown | | | | |
| Total | 0.71 | 0.26 | 0.23 | 0.21 |
| Impurities | | | | |

Conforms = Clear colorless to pale yellow solution, essentially free of visible particles

TABLE 11
Stability Results for Prototype B3 containing 20 mg/mL
Compound 1 in 25% v/v Dehydrated Alcohol, 37.5% v/v Polysorbate 80,
and 37.5% PEG 300 Drug Product Stored at 2-8° C.
Test ID

| | Time (Months) | | | |
|---|---|---|---|---|
| | 0 | 6 | 9 | 12 |
| Appearance | Conforms | Conforms | Conforms | Conforms |

Potency

| | | | | |
|---|---|---|---|---|
| % LC | 101.0 | 100.0 | 102.0 | 99.5 |

Related substances (% w/w)

| | | | | |
|---|---|---|---|---|
| Impurity A | <0.10 | <0.10 | <0.10 | <0.10 |
| Impurity B | <0.10 | <0.10 | <0.10 | <0.10 |
| Impurity C | 0.71 (0.23) | 0.61 (0.19) | 0.59 | 0.57 |
| Impurity D | <0.10 | <0.10 | <0.10 | <0.10 |
| Impurity E | <0.10 | <0.10 | <0.10 | <0.10 |
| Impurity F | <0.10 | <0.10 | 0.10 | 0.12 |
| Individual | <0.10 | <0.10 | <0.10 | <0.10 |
| Unknown | | | | |
| Total | 0.71 | 0.61 | 0.69 | 0.69 |
| Impurities | | | | |

Conforms = Clear colorless to pale yellow solution, essentially free of visible particles

TABLE 12
Stability Results for Prototype B4 containing 20 mg/mL
Compound 1 in 20% v/v Dehydrated Alcohol, 25% v/v Polysorbate 80,
and 55% PEG 300 Drug Product Stored at 40° C./75% RH
Test ID

| | Time (Months) | | |
|---|---|---|---|
| | 0 | 1 | 3 |
| Appearance | Conforms | Conforms | Conforms |
| Assay (% LC) | 101.5 | 101.0 | 100.0 |

Related substances (% w/w)

| | | | |
|---|---|---|---|
| Impurity A | <0.10 | <0.10 | <0.10 |
| Impurity B | <0.10 | <0.10 | <0.10 |
| Impurity C | 0.73 (0.23) | 0.58 (0.19) | 0.32 (0.10) |
| Impurity D | <0.10 | <0.10 | <0.10 |
| Impurity E | <0.10 | <0.10 | <0.10 |
| Impurity F | <0.10 | <0.10 | <0.10 |
| Individual | <0.10 | <0.10 | <0.10 |
| Unknown | | | |
| Total | 0.73 | 0.58 | 0.32 |
| Impurities | | | |

Conforms = Clear colorless to pale yellow solution, essentially free of visible particles

TABLE 13
Stability Results for Prototype B5 containing 10 mg/mL
Compound 1 in 85% v/v Polysorbate 80, and 15%
PEG 300 Drug Product Stored at 40° C./75% RH
Test ID

| | Time (Months) | | |
|---|---|---|---|
| | 0 | 1 | 3 |
| Appearance | Conforms | Conforms | Conforms |
| Assay (% LC) | 104.0 | 104.0 | 102.0 |

Related substances (% w/w)

| | | | |
|---|---|---|---|
| Impurity A | <0.10 | <0.10 | <0.10 |
| Impurity B | <0.10 | <0.10 | <0.10 |
| Impurity C | 0.68 (0.22) | 0.37 (0.12) | 0.12 (<0.10) |
| Impurity D | <0.10 | <0.10 | <0.10 |
| Impurity E | <0.10 | <0.10 | <0.10 |
| Impurity F | <0.10 | <0.10 | <0.10 |
| Individual | <0.10 | <0.10 | <0.10 |
| Unknown | | | |
| Total | 0.68 | 0.37 | 0.12 |
| Impurities | | | |

Conforms = Clear, colorless to pale yellow solution, essentially free of visible particles

### Experiment 7

### Cross-Over Pharmacokinetics Study of Formulations A and B

The group designation and related information were shown in Table 14. A stratified randomization scheme incorporating body weights was used to assign 5 female and 5 male monkeys into two treatment groups. All animals received two different formulations of compound 1 (Formulation A: Compound 1 at 1 mg/mL in 11.25% PEG300, 1.25% Tween 80, 87.5% D5W, and Formulation B: Compound 1 at 1 mg/mL in 1.6% PEG300, 1.4% Tween 80, 1% dehydrated alcohol, 96% D5W) at 5 mg/kg on Days 1 and 8, in a standard cross-over design. Animals in Group 1 (2 females and 3 males) received Formulation A on Day 1 and Formulation B on Day 8 while animals in Group 2 (3 females and 2 males) received Formulation B on Day 1 and Formulation A on Day 8.

Compound 1 was administered as a 1-h IV infusion (±2 minutes), in a volume of 5 mL/kg at a rate of approximately 0.083 mL/kg/min. Dose volumes were calculated based on body weights measured on the days prior to dosing.

The 1 mg/mL compound 1 dosing solutions were prepared on each day of dosing (Days 1 and 8) by slowly transferring the required amount of stock solutions of Formulation A (8 mg/mL compound 1 in 90% PEG300, 10% Tween 80) and Formulation B (25 mg/mL of compound 1 in 39.35% PEG300, 35% Tween 80, 25% dehydrated alcohol) to sterile containers containing appropriate amounts of D5W, and stirred for approximately 10 to 20 minutes. The resulting 1 mg/mL dosing solutions were sterile filtered using 0.22 μm polyvinylidene fluoride (PVDF) syringe filters prior to use.

Blood samples (approximately 1.0 mL) were collected by venipuncture from each restrained, non-sedated animal using butterfly infusions set and disposable syringes at predose, 0.25, 0.5, and 0.75 hour after the SOI, immediately prior to the EOI (within 3 minutes), and at 0.25, 0.5, 1, 2, 4, 8, 12, 18, and 24 hours after EOI.

Blood was transferred to tubes treated with sodium heparin and placed on wet ice, then centrifuged at 4° C. within approximately 30 minutes of collection to obtain plasma. Plasma samples were divided into two aliquots, transferred into cryovials, and stored at −60° C. or below. Remaining cell pellets were also stored at −60° C. or below after processing.

TABLE 14
Group Designation and Dose Levels

| | | | Dose | Dose Con- | Dose | Number | |
|---|---|---|---|---|---|---|---|
| Dosing | | Formu- | Level | centration | Volume | of Animals | |
| Day | Group | lation | (mg/kg) | (mg/mL) | (mL/kg) | Female | Male |
| 1 | 1 | A | 5 | 1 | 5 | 2 | 3 |
| | 2 | B | 5 | 1 | 5 | 3 | 2 |
| 8 | 1 | B | 5 | 1 | 5 | 2 | 3 |
| | 2 | A | 5 | 1 | 5 | 3 | 2 |

Formulation A: Compound 1 at 1 mg/mL in 11.25% PEG300, 1.25% Tween 80, 87.5% D5W
Formulation B: Compound 1 at 1 mg/mL in 1.6% PEG300, 1.4% Tween 80, 1% dehydrated alcohol, 96% D5W

### Summary of Sample Analysis Procedures

Plasma concentrations of compound 1 were determined using a qualified LC-MS/MS method. The assay was conducted with 25 μL of plasma; the lower limit of quantitation (LLOQ) was 1.00 ng/mL with a concentration range from 1.00 to 1000 ng/mL.

### Pharmacokinetic Data Analysis

The PK parameters for compound 1 were determined from the plasma concentration data using the Noncompartmental Analysis module in WinNonlin, version 5.2. This program analyzed data using the standard methods described by Gibaldi and Perrier. Nominal doses and sampling times were used. The predose sample results that were below the quantitation limit (BQL) were treated as zero for the calculations. The AUC values were estimated by the linear trapezoidal rule. The apparent half-life (t_{1/2}) was calculated as t_{1/2}=0.693/λ_{z} where λ_{z} was the elimination rate constant estimated from the regression of the terminal slope of the concentration versus time curve. At least three data points after the peak concentration on the terminal log-linear phase were used to determine λ_{z} and the coefficient of determination (r²) was required to be ≧0.85. If the acceptance criteria were not met, t_{1/2} was reported as not estimable (NE). If t_{1/2} was not estimable, AUC_{inf}, CL and volume of distribution (Vz or Vss) were reported as NE. AUC_{inf} was reported as not reportable (NR) if the ratio of AUCₜ/AUC_{inf} was ≦0.80.

### Statistical Analysis

Plasma concentrations as well as PK parameters for compound 1 with each formulation were summarized using descriptive statistics.

For evaluation of bioequivalency, statistical analyses were performed on the PK data set. A conventional two treatment, two period, two sequence, cross-over design was used. A parametric general linear model was applied to logarithmic transformations of each of Cmax, AUCt, and AUC_{inf}. Data were analyzed using a general linear mixed effect model (WinNonlin version 5.2) with factors included for subject, group (sequence), and day (period). Formulation A was defined as the reference formulation and Formulation B was defined as the test formulation. The comparison was made by calculating the ratio of Cmaxₜₑₛₜ/Cmax_{ref} and AUCₜₑₛₜ/AUC_{ref}: If both the lower and upper bounds of the 90% confidence interval (CI) for the ratio of geometric means of test formulation to reference formulation fell within 80-125%, then the bioequivalence of the test formulation and reference formulation was concluded.

### Results

Mean plasma concentrations of compound 1 at each sampling time following IV infusion to monkeys were reported in Table 15. The individual and mean PK parameters were presented in Tables 16 through 19.

Plasma concentrations of compound 1 at predose on both study days were BQL in any of the animals.

The plasma concentration of compound 1 increased rapidly during the infusion. The mean compound 1 Cmax values with Formulation A and Formulation B were 2670±435 ng/mL and 2860±593 ng/mL, respectively. Following the end of infusion, compound 1 was rapidly eliminated from plasma with the mean terminal t_{1/2} values of 9.4±1.6 h and 9.2±1.3 h with Formulations A and B, respectively. The mean AUC_{inf}, CL and Vss values were 3890±668 h·ng/mL and 3890±904 h·ng/mL, 1.32±0.24 L/h/kg and 1.34±0.28 L/h/kg, 3.06±0.71 L/kg and 3.09±0.61 L/kg with Formulations A and B, respectively.

Table 20 summarized the statistical analyses for compound 1 Cmax, AUCt and AUC_{inf}. The 90% confidence intervals for Cmax, AUCt and AUC_{inf} ratios were 99.2-114.2, 95.4-108.1, 93.6-112.4, respectively, based on natural logarithm (ln)-transformation. These confidence intervals were entirely contained within the bioequivalence limits of 80.00-125.00 and, thus, demonstrated that Formulation A was bioequivalent to Formulation B when compound 1 was dosed to monkeys.

TABLE 15
Mean Plasma Concentrations of Compound 1 Following
Intravenous Infusion (5 mg/kg) to Monkeys

| | | | | Cp (mg/mL) | | | |
|---|---|---|---|---|---|---|---|
| Formu- | | Male + Female | | Female Only | | Male Only | |
| lation | Time | Mean | SD | Mean | SD | Mean | SD |
| A | 0 | BQL | NA | BQL | NA | BQL | NA |
| | 0.25 | 1840 | 330 | 1750 | 322 | 1940 | 345 |
| | 0.5 | 2000 | 451 | 1950 | 407 | 2040 | 536 |
| | 0.75 | 2370 | 601 | 2170 | 766 | 2560 | 355 |
| | 1 | 2650 | 408 | 2500 | 499 | 2810 | 259 |
| | 1.25 | 780 | 194 | 674 | 162 | 886 | 174 |
| | 1.5 | 530 | 156 | 438 | 71.0 | 622 | 168 |
| | 2 | 386 | 111 | 313 | 76.1 | 459 | 93.7 |
| | 3 | 178 | 59.9 | 149 | 60.6 | 208 | 47.3 |
| | 5 | 63.0 | 21.7 | 50.1 | 15.7 | 75.9 | 19.9 |
| | 9 | 24.0 | 8.30 | 18.7 | 3.31 | 29.2 | 8.66 |
| | 13 | 14.2 | 4.03 | 11.4 | 2.12 | 17.0 | 3.56 |
| | 19 | 8.32 | 2.03 | 7.43 | 1.84 | 9.20 | 1.98 |
| | 25 | 5.98 | 1.21 | 5.79 | 1.26 | 6.17 | 1.27 |
| B | 0 | BQL | NA | BQL | NA | BQL | NA |
| | 0.25 | 1790 | 193 | 1740 | 257 | 1830 | 114 |
| | 0.5 | 2230 | 524 | 2050 | 506 | 2410 | 527 |
| | 0.75 | 2650 | 490 | 2370 | 445 | 2930 | 381 |
| | 1 | 2820 | 560 | 2540 | 539 | 3100 | 468 |
| | 1.25 | 761 | 259 | 596 | 71.3 | 927 | 277 |
| | 1.5 | 546 | 175 | 463 | 54.8 | 630 | 220 |
| | 2 | 336 | 98.0 | 261 | 20.1 | 410 | 85.6 |
| | 3 | 180 | 65.2 | 142 | 42.0 | 218 | 64.4 |
| | 5 | 62.6 | 25.7 | 53.5 | 20.6 | 71.7 | 29.2 |
| | 9 | 22.0 | 6.11 | 21.8 | 6.92 | 22.2 | 6.01 |
| | 13 | 19.5 | 13.3 | 13.1 | 3.97 | 25.9 | 16.7 |
| | 19 | 8.68 | 2.46 | 7.59 | 2.10 | 9.77 | 2.50 |
| | 25 | 7.18 | 2.63 | 6.13 | 2.58 | 8.24 | 2.48 |

BQL: below the quantitation limit (1.00 ng/mL)
NA: not applicable

TABLE 16
Individual Pharmacokinetic Parameters for Compound 1 in Monkeys

| | | | t_{1/2} | Tmax | Cmax | AUCt | AUC_{inf} | CL | Vz | Vss |
|---|---|---|---|---|---|---|---|---|---|---|
| Gender | Day | Form. | (h) | (h) | (ng/mL) | (h · ng/mL) | (h · ng/mL) | (L/h/kg) | (L/kg) | (L/kg) |
| Female | 8 | A | NE | 1.0 | 2330 | 2930 | NE | NE | NE | NE |
| | 1 | B | 10.9 | 1.0 | 2730 | 3170 | 3230 | 1.550 | 24.3 | 2.99 |
| Female | 1 | A | 10.9 | 0.7 | 3190 | 4550 | 4650 | 1.08 | 16.9 | 2.37 |
| | 8 | B | 8.6 | 1.0 | 3300 | 4080 | 4160 | 1.20 | 14.9 | 2.48 |
| Female | 1 | A | 9.9 | 1.0 | 2970 | 3670 | 3740 | 1.34 | 19.1 | 2.54 |
| | 8 | B | NE | 1.0 | 2520 | 3110 | NE | NE | NE | NE |
| Female | 8 | A | 12.1 | 1.1 | 2140 | 2960 | 3050 | 1.64 | 28.6 | 4.12 |
| | 1 | B | 8.8 | 0.8 | 1880 | 2860 | 2920 | 1.71 | 21.7 | 3.74 |
| Female | 8 | A | 9.6 | 1.3 | 1970 | 3040 | 3130 | 1.60 | 22.3 | 4.24 |
| | 1 | B | 7.8 | 1.1 | 2300 | 3340 | 3410 | 1.47 | 16.4 | 3.92 |
| Male | 1 | A | 7.9 | 1.1 | 2450 | 4150 | 4210 | 1.19 | 13.6 | 2.64 |
| | 8 | B | 9.2 | 1.0 | 2650 | 3590 | 3680 | 1.36 | 18.1 | 3.39 |
| Male | 1 | A | 8.2 | 1.0 | 2720 | 2980 | 3020 | 1.65 | 19.5 | 3.44 |
| | 8 | B | 9.0 | 0.8 | 2910 | 3270 | 3330 | 1.50 | 19.5 | 3.05 |
| Male | 8 | A | 6.8 | 0.8 | 3110 | 4380 | 4440 | 1.13 | 11.1 | 2.43 |
| | 1 | B | 8.1 | 1.1 | 3810 | 5420 | 5520 | 0.91 | 10.6 | 2.09 |
| Male | 1 | A | 9.3 | 1.0 | 3050 | 4440 | 4540 | 1.10 | 14.8 | 2.8 |
| | 8 | B | 11.3 | 0.8 | 3310 | 4740 | 4900 | 1.02 | 16.6 | 3.04 |
| Male | 8 | A | 10.1 | 1.0 | 2740 | 4100 | 4210 | 1.19 | 17.3 | 2.96 |
| | 1 | B | NE | 1.0 | 3140 | 4370 | NE | NE | NE | NE |

NE: Not estimable due to insufficient characterization of terminal phases

TABLE 17
Mean Pharmacokinetic Parameters for Compound 1 in Female and Male Monkeys

| | | | t1/2 | Tmax | Cmax | AUCt | AUCinf | CL | Vz | Vss |
|---|---|---|---|---|---|---|---|---|---|---|
| Form | Gender | Day | (h) | (h) | (ng/mL) | (h · ng/mL) | (h · ng/mL) | (L/h/kg) | (L/kg) | (L/kg) |
| A | Female | 8 | NE | 1.0 | 2330 | 2930 | NE | NE | NE | NE |
| | Female | 1 | 10.9 | 0.7 | 3190 | 4550 | 4650 | 1.08 | 16.9 | 2.37 |
| | Female | 1 | 9.9 | 1.0 | 2970 | 3670 | 3740 | 1.34 | 19.1 | 2.54 |
| | Female | 8 | 12.1 | 1.1 | 2140 | 2960 | 3050 | 1.64 | 28.6 | 4.12 |
| | Female | 8 | 9.6 | 1.3 | 1970 | 3040 | 3130 | 1.60 | 22.3 | 4.24 |
| | Male | 1 | 7.9 | 1.1 | 2450 | 4150 | 4210 | 1.19 | 13.6 | 2.64 |
| | Male | 1 | 8.2 | 1.0 | 2720 | 2980 | 3020 | 1.65 | 19.5 | 3.44 |
| | Male | 8 | 6.8 | 0.8 | 3110 | 4380 | 4440 | 1.13 | 11.1 | 2.43 |
| | Male | 1 | 9.3 | 1.0 | 3050 | 4440 | 4540 | 1.10 | 14.8 | 2.80 |
| | Male | 8 | 10.1 | 1.0 | 2740 | 4100 | 4210 | 1.19 | 17.3 | 2.96 |
| | | Mean | 9.4 | 1.0 | 2670 | 3720 | 3890 | 1.32 | 18.1 | 3.06 |
| | | SD | 1.6 | 0.2 | 435 | 661 | 668 | 0.24 | 5.16 | 0.71 |
| | | CV % | 17 | 17 | 16 | 18 | 17 | 18 | 28 | 23 |
| | | N | 9 | 10 | 10 | 10 | 9 | 9 | 9 | 9 |
| B | Female | 1 | 10.9 | 1.0 | 2730 | 3170 | 3230 | 1.55 | 24.3 | 2.99 |
| | Female | 8 | 8.6 | 1.0 | 3300 | 4080 | 4160 | 1.20 | 14.9 | 2.48 |
| | Female | 8 | NE | 1.0 | 2520 | 3110 | NE | NE | NE | NE |
| | Female | 1 | 8.8 | 0.8 | 1880 | 2860 | 2920 | 1.71 | 21.7 | 3.74 |
| | Female | 1 | 7.8 | 1.1 | 2300 | 3340 | 3410 | 1.47 | 16.4 | 3.92 |
| | Male | 8 | 9.2 | 1.0 | 2650 | 3590 | 3680 | 1.36 | 18.1 | 3.39 |
| | Male | 8 | 9.0 | 0.8 | 2910 | 3270 | 3330 | 1.50 | 19.5 | 3.05 |
| | Male | 1 | 8.1 | 1.1 | 3810 | 5420 | 5520 | 0.91 | 10.6 | 2.09 |
| | Male | 8 | 11.3 | 0.8 | 3310 | 4740 | 4900 | 1.02 | 16.6 | 3.04 |
| | Male | 1 | NE | 1.0 | 3140 | 4370 | NE | NE | NE | NE |
| | | Mean | 9.2 | 1.0 | 2860 | 3800 | 3890 | 1.34 | 17.8 | 3.09 |
| | | SD | 1.3 | 0.1 | 593 | 849 | 904 | 0.28 | 4.21 | 0.61 |
| | | CV % | 14 | 13 | 21 | 22 | 23 | 21 | 24 | 20 |
| | | N | 8 | 10 | 10 | 10 | 8 | 8 | 8 | 8 |

NE: Not estimable due to insufficient characterization of terminal phases

TABLE 18
Mean Pharmacokinetic Parameters for Compound 1 in Female Monkeys

| | | t_{1/2} | Tmax | C max | AUCt | AUC_{inf} | CL | Vz | Vss |
|---|---|---|---|---|---|---|---|---|---|
| Form | Day | (h) | (h) | (ng/mL) | (h · ng/mL) | (h · ng/mL) | (L/h/kg) | (L/kg) | (L/kg) |
| A | 8 | NE | 1.0 | 2330 | 2930 | NE | NE | NE | NE |
| | 1 | 10.9 | 0.7 | 3190 | 4550 | 4650 | 1.08 | 16.9 | 2.37 |
| | 1 | 9.9 | 1.0 | 2970 | 3670 | 3740 | 1.34 | 19.1 | 2.54 |
| | 8 | 12.1 | 1.1 | 2140 | 2960 | 3050 | 1.64 | 28.6 | 4.12 |
| | 8 | 9.6 | 1.3 | 1970 | 3040 | 3130 | 1.6 | 22.3 | 4.24 |
| | Mean | 10.6 | 1.0 | 2520 | 3430 | 3640 | 1.42 | 21.7 | 3.32 |
| | SD | 1.1 | 0.2 | 533 | 696 | 739 | 0.26 | 5.09 | 1.00 |
| | CV % | 11 | 21 | 21 | 20 | 20 | 18 | 23 | 30 |
| | N | 4 | 5 | 5 | 5 | 4 | 4 | 4 | 4 |
| B | 1 | 10.9 | 1.0 | 2730 | 3170 | 3230 | 1.55 | 24.3 | 2.99 |
| | 8 | 8.6 | 1.0 | 3300 | 4080 | 4160 | 1.20 | 14.9 | 2.48 |
| | 8 | NE | 1.0 | 2520 | 3110 | NE | NE | NE | NE |
| | 1 | 8.8 | 0.8 | 1880 | 2860 | 2920 | 1.71 | 21.7 | 3.74 |
| | 1 | 8 | 1.1 | 2300 | 3340 | 3410 | 1.47 | 16.4 | 3.9 |
| | Mean | 9.0 | 1.0 | 2550 | 3310 | 3430 | 1.48 | 19.3 | 3.28 |
| | SD | 1.3 | 0.1 | 526 | 463 | 527 | 0.21 | 4.42 | 0.67 |
| | CV % | 15 | 11 | 21 | 14 | 15 | 14 | 23 | 20 |
| | N | 4 | 5 | 5 | 5 | 4 | 4 | 4 | 4 |

NE: Not estimable due to insufficient characterization of terminal phases

TABLE 19
Mean Pharmacokinetic Parameters of Compound 1 in Male Monkeys

| | | t_{1/2} | Tmax | Cmax | AUCt | AUC_{inf} | CL | Vz | Vss |
|---|---|---|---|---|---|---|---|---|---|
| Form | Day | (h) | (h) | (ng/mL) | (h · ng/mL) | (h · ng/mL) | (L/h/kg) | (L/kg) | (L/kg) |
| A | 1 | 7.9 | 1.1 | 2450 | 4150 | 4210 | 1.19 | 13.6 | 2.64 |
| | 1 | 8.2 | 1.0 | 2720 | 2980 | 3020 | 1.65 | 19.5 | 3.44 |
| | 8 | 6.8 | 0.8 | 3110 | 4380 | 4440 | 1.13 | 11.1 | 2.43 |
| | 1 | 9.3 | 1.0 | 3050 | 4440 | 4540 | 1.1 | 14.8 | 2.8 |
| | 8 | 10.1 | 1.0 | 2740 | 4100 | 4210 | 1.19 | 17.3 | 2.96 |
| | Mean | 8.5 | 1.0 | 2810 | 4010 | 4080 | 1.25 | 15.3 | 2.85 |
| | SD | 1.3 | 0.1 | 269 | 594 | 612 | 0.23 | 3.26 | 0.38 |
| | CV % | 15 | 11 | 10 | 15 | 15 | 18 | 21 | 13 |
| | N | 5 | 5 | 5 | 5 | 5 | 5 | 5 | 5 |
| B | 8 | 9.2 | 1.0 | 2650 | 3590 | 3680 | 1.36 | 18.1 | 3.39 |
| | 8 | 9.0 | 0.8 | 2910 | 3270 | 3330 | 1.50 | 19.5 | 3.05 |
| | 1 | 8.1 | 1.1 | 3810 | 5420 | 5520 | 0.91 | 10.6 | 2.09 |
| | 8 | 11.3 | 0.8 | 3310 | 4740 | 4900 | 1.02 | 16.6 | 3.04 |
| | 1 | NE | 1.0 | 3140 | 4370 | NE | NE | NE | NE |
| | Mean | 9.4 | 0.9 | 3160 | 4280 | 4360 | 1.20 | 16.2 | 2.89 |
| | SD | 1.4 | 0.1 | 438 | 868 | 1030 | 0.28 | 3.92 | 0.56 |
| | CV % | 13 | 14 | 14 | 20 | 13 | 13 | 13 | 13 |
| | N | 4 | 5 | 5 | 5 | 4 | 4 | 4 | 4 |

NE: Not estimable due to insufficient characterization of terminal phases

TABLE 20
Summary of Statistical Analysis on
Compound 1 Cmax, AUCt and AUC_{inf}

| | | Geometric | Ratio of | 90% | |
|---|---|---|---|---|---|
| | | Least-Square | Form B/ | Confidence | |
| Parameter | Formulation | Mean | Form A | Interval | p-value |
| Cmax | A | 2634.7 | 106.4 | 99.2-114.2 | 0.1398 |
| | B | 2803.4 | | | |
| AUCt | A | 3662.4 | 101.6 | 95.4-108.1 | 0.6597 |
| | B | 3719.2 | | | |
| AUC_{inf} | A | 3755.6 | 102.6 | 93.6-112.4 | 0.6343 |
| | B | 3852.1 | | | |

### Conclusion

The plasma concentration—time profiles of compound 1 were nearly superimposable between the two formulations tested. With both formulations, plasma concentrations of compound 1 steadily increased during infusion and achieved Cmax at during the infusion or EOI, then declined quickly.

Mean PK parameters, including t_{1/2}, Cmax, Tmax, AUC_{inf}, CL and Vss were very similar between the two formulations, and based on the statistical analysis for Cmax, AUCt and AUC_{inf}, it was demonstrated that Formulations A and B were bioequivalent for compound 1 in monkeys. Therefore, there would be no PK alterations when Formulation A is replaced by Formulation B in clinic.

In summary, the novel formulation has many advantages such as complete miscibility of the drug product solution, substantially lowered viscosity, and significantly increased infusion solution physical stability up to 10 hours. Moreover, the novel formulation could be stored in a single vial containing 200 mg/m² dose and had excellent physical and chemical stability up to 1 year.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

1. A pharmaceutical composition comprising a pharmaceutically acceptable organic solvent;
a pharmaceutically acceptable surfactant; and a compound according to formula (IV): 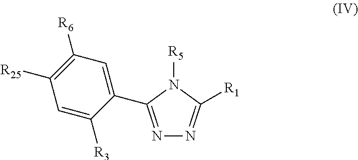
or a tautomer, or a pharmaceutically acceptable salt thereof, wherein
R₁ and R₃ are, independently, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;
R₅ is an optionally substituted heteroaryl or an optionally substituted 8 to 14 membered aryl;
R₆, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, —SP(O)(OR₇)₂, —NR₇C(O)R₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —NR₇S(O)ₚR₇, —C(NR₈)OR₇, or —S(O)ₚR₇; and
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₂₅ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂₆ is a C1-C6 alkyl;
p, for each occurrence, is, independently, 0, 1 or 2;
m, for each occurrence, is independently, 1, 2, 3, or 4;
wherein the organic solvent is selected from the group consisting of polyethylene glycol, dimethyl sulfoxide, N-methylpyrolidinone, and glycerine; and
wherein the surfactant is selected from the group consisting of polysorbate 80, cremophor, and polyvinyl povidone.

2-19. (canceled)

20. The pharmaceutical composition of claim 1, wherein the Hsp90 inhibitor is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

21. The pharmaceutical composition of claim 1, further comprising a pharmaceutically acceptable co-solvent.

22-27. (canceled)

28. The pharmaceutical composition of claim 1, wherein the organic solvent is PEG-300, the surfactant is polysorbate 80, and the Hsp90 inhibitory compound is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

29. The pharmaceutical composition of claim 28, wherein the v/v ration of PEG-300 and polysorbate 80 is about 9:1, and the concentration of the Hsp90 inhibitory compound is about 8 mg/mL.

30. The pharmaceutical composition of claim 21, wherein the organic solvent is PEG-300, the surfactant is polysorbate 80, the co-solvent is dehydrated alcohol and the Hsp90 inhibitory compound is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

31. The pharmaceutical composition of claim 30, wherein the v/v/v ratio of PEG-300, polysorbate 80 and dehydrated alcohol is about 39.35/35/25, and the concentration of the Hsp90 inhibitory compound is about 25 mg/mL.

32-33. (canceled)

34. An admixture comprising the composition of claim 28 and a solution selected from normal saline and D5W.

35. (canceled)

36. An admixture comprising the composition of claim 30 and a solution selected from normal saline and D5W.

37. (canceled)

38. A method of treating a subject in need thereof, comprising administering to the subject a pharmaceutical composition of claim 1 at a dose of about 200 mg/m².

39. The method of claim 38, wherein the pharmaceutical composition is about 39.35% (v/v) PEG-300, about 35% (v/v) polysorbate 80, about 25% (v/v) dehydrated alcohol and the Hsp90 inhibitory compound is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof, in a concentration of about 25 mg/mL.

40. The method of claim 38, wherein the pharmaceutical composition is administered parentally.

41. The method of claim 40, wherein the pharmaceutical composition is administered with an in-dwelling silicone catheter.

42. The method of claim 40, wherein the pharmaceutical composition is administered via peripheral venous access.

43. The method of claim 40, wherein the pharmaceutical composition is administered via silicone peripherally inserted central catheter.

44. The pharmaceutical composition of claim 20, further comprising a pharmaceutically acceptable co-solvent.

45. The method of claim 39, wherein the pharmaceutical composition is administered parentally.

46. The method of claim 45, wherein the pharmaceutical composition is administered with an in-dwelling silicone catheter.

47. The method of claim 45, wherein the pharmaceutical composition is administered via peripheral venous access.

48. The method of claim 45, wherein the pharmaceutical composition is administered via silicone peripherally inserted central catheter.
